(19) United States
(12) Patent Application Publication
(10) Pub. No.: US 2026/0158151 A1
(43) Pub. Date: Jun. 11, 2026

(54) PEG-PPS NANOCARRIER DELIVERY OF THE RAS/RAP1 SPECIFIC ENDOPEPTIDASE

(71) Applicant: Northwestern University, Evanston, IL (US)

(72) Inventors: Karla Jean Fullner Satchell, Evanston, IL (US); Evan Alexander Scott, Evanston, IL (US); Taylor Elizabeth Escher, Evanston, IL (US); Yuan Qian, Evanston, IL (US)

(21) Appl. No.: 19/150,127

(22) PCT Filed: Jan. 23, 2024
(86) PCT No.: PCT/US2024/012563

§ 371 Date: Jul. 22, 2025

Related U.S. Application Data
(60) Provisional application No. 63/481,145, filed on Jan. 23, 2023.

(51) Int. Cl. A61K 47/64; A61K 9/00; A61K 31/7105; A61K 31/711; A61K 38/46; A61K 47/60; A61K 47/69; A61P 35/00; C12N 15/88

(52) U.S. Cl. CPC A61K 47/6455; A61K 9/0019; A61K 31/7105; A61K 31/711; A61K 38/46; A61K 47/60; A61K 47/6935; A61P 35/00; C12N 15/88; C12Y 304/00

(57) ABSTRACT

The present disclosure describes a nanocarrier system comprising PEG-b-PPS copolymers conjugated to dendritic peptides for delivery of a nucleic acid encoding RAS/RAP1-specific endopeptidase (RRSP) to a cell. Pharmaceutical compositions comprising the system and methods for treating cancer using the system are also disclosed herein.

### CROSS-REFERENCE TO RELATED APPLICATIONS

This application claims priority to U.S. Provisional Application No. 63/481,145 filed on Jan. 23, 2023, the content of which is incorporated by reference in its entirety.

### REFERENCE TO AN ELECTRONIC SEQUENCE LISTING

The contents of the electronic sequence listing (702581.02455.xml; Size: 26,044 bytes; and Date of Creation: Jan. 23, 2024) is herein incorporated by reference in its entirety.

### BACKGROUND OF THE INVENTION

Tumor cells express three major RAS genes that control cell proliferation in response to growth factor stimulation. When these genes acquire mutations, they can become constitutively active and drive malignant transformation. Historically, HRAS has been the most studied of the RAS genes, but KRAS is the most frequently mutated (85%), followed by NRAS (11%) and HRAS (4%). RAS mutations are considered cancer drivers, but they are also important for maintaining survival of the tumors, making RAS an attractive therapeutic target. Despite the high prevalence of RAS mutations in cancers, targeting RAS therapeutically has proven difficult. RAS has even been termed “undruggable” because it lacks well-defined binding pockets and the high levels of cellular GTP outcompete small molecules at its GTP-binding site. Further, the existence of multiple RAS genes and splice variants (isoforms) presents another challenge as the different RAS proteins are differentially expressed depending on cell type and cell lines vary considerably in how dependent they are on the RAS oncoproteins.

Currently, the only FDA-approved therapies specifically directed toward RAS have focused on targeting cancers with specific KRAS mutations, which are only present in a small percentage of all cancers. New therapeutic strategies under development are the “RAS Degraders”, which specifically target RAS for proteolytic turnover and result in lowered levels of RAS in the cell and thereby can be useful in nearly all tumors. In addition, “pan-RAS” degraders that target all forms of RAS in the cell can drive cells to be devoid of RAS and thereby stop cell proliferation.

A well-studied intracellular RAS Degrader is the RAS/Rap1 specific endopeptidase, or RRSP. Originally termed DUF5, RRSP is a bacterial cytotoxic effector domain from the multifunctional-autoprocessing repeats-in-toxin (MARTX) toxin. RRSP was originally discovered in the pathogen Vibrio vulnificus, but active toxin has also been studied from Photorhadus luminescens and from Aeromonas hydrophila. The x-ray crystal structure of ˜56 kDa protein RRSP revealed it is a multidomain protein with a membrane targeting N-terminal domain and a novel C-terminal protease domain related to the TIKI family of eukaryotic enzymes. The active site is comprised of a conserved glutamic acid and histidine residues.

RRSP site-specifically cleaves RAS and its close homologue RAP1 between residues Tyrosine-32 and Aspartic acid-33 within the Switch I region, which is crucial for RAS interaction with RAF kinases in the RAS-ERK signaling axis. RRSP does not cleave other closely related GTPases, but can cleave all three of the major RAS isoforms (H, N, and K), the most common oncogenic RAS mutations including G12C, G12D, G12V, G13D, and Q61R, and both GTP and GDP-bound RAS. Within cells, RRSP degradation of RAS leads to G1 cell cycle arrest that can progress to apoptosis, senescence, and loss of cell proliferation depending on the cell line. The protein also can induce cell cycle arrest by binding to proteins when overexpressed in cells, independent of RAS processing. The cells then fail to proliferate. RRSP is capable of reducing cell growth in more than 80% of all cells lines where it has been tested, including most of the cells in the NCI-60 panel that includes leukemia, Non-small cell lung carcinoma, colorectal carcinoma, central nervous system cancers, melanoma, ovarian cancers, renal cancers, and breast cancer. RRSP is also active against pancreatic cancers. In vivo studies showed that RRSP can reduce breast and colon tumor growth and induced regression of pancreatic cancer patient-derived xenografts (PDXs).

The major limitation to use of RRSP as a cancer therapeutic is that the protein naturally occurs as a single domain of >5000 amino acid bacterial toxin protein and the domain itself is not able to transit across the cell plasma membrane without the remaining portions of the larger toxin. One strategy to deliver RRSP to cells has been to fuse RRSP to domains of other bacterial toxins that facilitate translocation into cells via receptor binding and endocytosis followed by the chimeric toxin translocation across the endocytic vacuolar membrane. This has been developed as a fusion of RRSP to diphtheria toxin and fusion of RRSP to anthrax toxin lethal factor. However, to achieve a more flexible delivery platform, it is advantageous to circumvent the need for purified RRSP protein to be translocated across the membrane and instead delivery a nucleic acid to the cell that will then express the RRSP domain within cells.

### SUMMARY OF THE INVENTION

The present invention comprises a system for delivery of a nucleic acid to a cell and methods of doing the same. In an aspect, provided herein is a system for delivery of a nucleic acid encoding RAS/RAP1-specific endopeptidase (RRSP) to a cell, the system comprising: a nanocarrier comprising a poly(ethylene glycol)-block-poly(propylene sulfide) copolymer (PEG-b-PPS) conjugated with a dendritic-specific branched cationic peptide (DP)(PPDP); and a nucleic acid encoding RRSP.

In embodiments, the PEG-b-PPS has a PEG weight fraction of between about 0.05 and about 0.50. In embodiments, the PEG-b-PPS is PEG₁₇-b-PPS₈₀. In embodiments, the mass ratio (w/w) of PPDP:nucleic acid is between about 15:1 and about 120:1.

In embodiments, the nucleic acid is an mRNA comprising a sequence having at least 90% identity to SEQ ID NO: 4.

In embodiments, the RRSP is a catalytically inactive form of RRSP. In embodiments, the nucleic acid is an mRNA comprising a sequence having at least 90% identity to SEQ ID NO: 3

In embodiments, the nucleic acid is a DNA comprising a sequence having at least 90% identity to SEQ ID NO: 7.

In another aspect, provided herein is a pharmaceutical composition comprising any of the systems described herein.

In another aspect, provided herein is a method of delivering a nucleic acid encoding RRSP to a cell, the method comprising contacting the cell with any of the systems described herein. In embodiments, the cell is a mammalian cell. In embodiments, the cell is a non-phagocytic cell. In embodiments, the cell is a cancer cell.

In embodiments, the nucleic acid comprises a DNA encoding RRSP and further comprises a promoter, wherein the promoter is a cell-specific promoter. In embodiments, the cell-specific promoter is a cancer-specific promoter. In embodiments, the cell is selected from a lung cancer cell, a colon cancer cell, a pancreatic cancer cell, a skin cancer cell, and a breast cancer cell.

In another aspect, provided herein is a method of treating a cancer in a subject in need thereof, the method comprising administering to the subject a therapeutically effective amount of any of the systems or pharmaceutical compositions described herein. In embodiments, the cancer is selected from lung cancer, colon cancer, pancreatic cancer, skin cancer, and breast cancer.

In embodiments, the nucleic acid comprises a DNA encoding RRSP and further comprises a promoter, wherein the promoter is a cell-specific promoter. In embodiments, the cancer is characterized by an increased rate of RAS mutation or increased signal flow through RAS.

In embodiments, the pharmaceutical composition is administered intratumorally. In embodiments, the pharmaceutical composition is administered systemically.

### BRIEF DESCRIPTION OF THE DRAWINGS

Non-limiting embodiments of the present invention will be described by way of example with reference to the accompanying Figures, which are schematic and are not intended to be drawn to scale. In the Figures, each identical or nearly identical component illustrated is typically represented by a single numeral. For purposes of clarity, not every component is labeled in every Figure, nor is every component of each embodiment of the invention shown where illustration is not necessary to allow those of ordinary skill in the art to understand the invention.

FIGS. 1A-1E. PPDP2 nanoparticles deliver gfp-mRNA and mCherry-mRNA into pancreatic cancer cells (A) Schematic of nanoparticle chemistry. (B) Representative fluorescent images of PANC-1 cells after transfection of cells with 1 μg of gfp-mRNA alone (top), PPDP2 alone (1:40 w/v %) (second row), gfp-mRNA with PPDP2 (third row) or gfp-mRNA with Lipofectamine MessengerMAX (Lipo) (bottom row). (C) Quantification of the fraction of GFP positive cells (green) from five imaged frames are shown as a histogram. (D) Representative Western blot and quantification of mCherry levels from PANC-1 xenografts injected with PPDP2 alone or PPDP2-mCherry. (E) mCherry IHC staining from PANC-1 xenografts injected with PPDP2 alone or PPDP2-mCherry. P values were calculated using a one-way ANOVA and Dunnett's multiple comparisons test, *p<0.05, **p<0.01.

FIGS. 2A-2E. PPDP2-rrsp reduces levels of RAS and impacts cell proliferation in pancreatic cancer cells. (A) Representative Western blot and quantification of uncleaved/cleaved RAS and pERK levels of PANC-1 cells transfected for 24 hours with either vehicle only, PPDP2, PPDP2 nanoparticles loaded with rrsp mRNA or rrsp mixed with Lipo as indicated. (B) Representative image and quantification of crystal violet-stained colonies from PANC-1 cells treated with PPDP2, PPDP2 nanoparticles loaded with rrsp mRNA or rrsp mixed with Lipo as indicated. (C-D) Representative Western blot and quantification of uncleaved/cleaved RAS levels of PANC-1 cells treated with 5.5 μg of rrsp, indicative of dose for following in vivo experiment for 24 h. (E) Caspase Glo 3/7 luminescence activity of PANC-1 cells were treated with 5.5 μg of rrsp delivered with PPDP2. Data are presented as mean±SEM with n≥5. P values were calculated using a one-way ANOVA and Dunnett's multiple comparisons test, *p<0.05, **p<0.01, ***p<0.001.

FIGS. 3A-3F. PPDP2-rrsp growth of pancreatic tumors. (A) Tumor volume measured every other day, excluding weekends. Day 0 indicates day of first treatment with 0.25 mg/kg dose three times per week. (B) Images of resected tumors on Day 30. (C) Tumor weight, (D) tumor volume and (E) change in volume compared to first day of treatment, normalized to 0, on Day 30. (F) Weight of mice measured every other day. Data were presented as mean±SEM with n=5. P values were calculated using a one-way ANOVA and Dunnett's multiple comparisons test, *p<0.05, **p<0.01, ***p<0.001.

FIGS. 4A-4B. PPDP2-rrsp reduces levels of RAS and impacts cell proliferation in PANC-1 xenograft tumors. (A) H&E, Masson's trichrome, and IHC staining with anti-cytokeratin 19, anti-Ki-67, anti-pan-RAS, anti-Phospho-p44/42 MAPK, and anti-mCherry antibodies. (B) ImageJ was used to quantify intensity of brown staining. Data were presented as mean±SEM with n≥3. P values were calculated using a one-way ANOVA and Dunnett's multiple comparisons test, assuming equal distribution *p<0.05, **p<0.01, ***p<0.001.

FIGS. 5A-5C. PPDP2-rrsp does not reduce distant tumors. When the primary tumors (2 per mouse-one on each flank) averaged 80-120 mm³, mice were grouped into 2 groups of 5 mice and treatments were initiated. One tumor per mouse was treated (A) and while the other was not treated (B). The first group received PPDP2 nanoparticles alone (equivalent polymer dose) (circles), the second received 0.25 mg/kg of rrsp-PPDP2 (triangles). Treatments were only given IT at primary tumor. Tumor volume was measured every other day, excluding weekends. Day 0 indicates day of first treatment. P values were calculated using a one-way ANOVA and Dunnett's multiple comparisons test, assuming equal distribution ***p<0.001, NS=not significant.

FIGS. 6A-6D. Structural model of RRSP in complex with KRAS suggests extensive contact sites. (A) Space filling AlphaFold2-generated model of RRSP (MARTX toxin aa 3594-4078) in complex with KRAS (aa 1-175). RRSP C1 membrane localization domain (MLD, white), C2A (dark blue), C2B (medium blue), and KRAS (pink). Switch 1 (Sw1) is shown only as a ribbon in magenta with D32 and Y33 as sticks in yellow. Catalytic residues are colored lime green. Note that the Sw1 is pulled into the active site of RRSP. (B) Ribbon cartoon of RRSP (with C1 MLD removed) with backbone colored as in Panel A. Residues mapped as binding to KRAS are colored (residues that bind C2A are yellow and that bind C2B are magenta) (C) Residues in KRAS mapped as binding to C2A are colored orange and to C2B are colored green. Scissile bond is marked with triangle. (D) Alignment of all RAS sequences experimentally validated as successfully cleaved by RRSP. RALA:RAS is a chimera with 4 aa changes that alters the noncleaved RalA Sw1 to match the KRAS Sw1 (changed residues outlined) and is cleaved with equal efficiency as KRAS. 27 residues mapped as binding RRSP C2A are indicated with a hashtag (#) and to RRSP C2B as an asterisk (*). Sw1 and Sw2 are marked in panels C and D. Sequences from top down are SEQ ID NOs: 9-13.

FIGS. 7A-7D. PPDP2 loading of rrsp mRNA. (A) Z-average size of PPDP2 alone and PPDP2 loaded with rrsp mRNA. (B) Polydispersity index of PPDP2 alone and PPDP2 loaded with rrsp mRNA. (C) Zeta potential of PPDP2 alone and PPDP2 loaded with rrsp mRNA. (D) Gel electrophoresis of PPDP2 alone and PPDP2 loaded with rrsp mRNA forming nanocomplexes at 35:1 wt. ratio.

FIGS. 8A-8B. rrsp mRNA causes cell death in mouse pancreatic KPC cells. (A) Dose response to KPC cells transfected with six different doses of rrsp mRNA. (B) Relative growth after treatment with 1 or 10 μg of rrsp mRNA. (compared to PBS control) from time-lapse images taken 24-, 48-, or 72-hours following transfection.

FIGS. 9A-9C. PPDP2-rrsp reduces levels of RAS and impacts cell proliferation in KPC cells. (A) Representative fluorescent images of KPC cells after transfection of cells with 1 μg of egfp-mRNA alone (top), PPDP2 alone (1:40 w/v %) (second row), egfp-mRNA with PPDP2 (third row) or egfp-mRNA with Lipofectamine MessengerMAX (Lipo) (bottom row). (B) Representative Western blot and quantification of uncleaved/cleaved RAS and pERK levels of KPC cells transfected for 24 hours with either vehicle only, PPDP2, PPDP2 nanoparticles loaded with rrsp mRNA or rrsp mixed with Lipo as indicated. (C) Representative image and quantification of crystal violet-stained colonies from KPC cells treated with PPDP2, PPDP2 nanoparticles loaded with rrsp mRNA or rrsp mixed with Lipo as indicated. Data are presented as mean±SEM with n≥5. P values were calculated using a one-way ANOVA and Dunnett's multiple comparisons test, *p<0.05, **p<0.01, ***p<0.001.

FIGS. 10A-10C. HCT-116 cells transfected with rrsp-mRNA inhibits proliferation and reduces RAS expression. (A) GFP fluorescence of HCT-116 cells following mRNA transfection with synthetic egfp capped mRNA. (B) Representative images of HCT-116 cells transfected with rrsp mRNA transfected after 24 hours. (C) Western blot of RAS band disappearance following transfection.

FIGS. 11A-11B. rrsp mRNA is expressed in tumor tissue and produces functional RRSP protein. (A-B) Representative Western blot and quantification of HA, uncleaved/cleaved RAS, pERK, ERK, from protein extracts from frozen tissues.

FIG. 12. PPDP2+/−rrsp mRNA do not alter tissue architecture. (A) H&E-stained tissue from mice injected with PPDP2 alone, PPDP2-rrsp, and PPDP2-rrsp* or PPDP2-mCherry from heart, kidney, liver, lungs, and spleen.

FIGS. 13A-13E Minimum tolerated dose and off-target effects of RRSP protein delivery to all cells. (A) Survival curve from mice treated with vehicle, RRSP-DT_{B} or RRSP*-DT_{B} treated DTR mice at indicated dose. (B) Median weights across all groups are reported along with occurrence of deaths. (C) H&E-stained tissue from heart, kidney, liver, lungs, and spleen from vehicle and 0.5 mg/kg RRSP-DT_{B}. (D) Western blot assay of HB-EGF/DTR receptor expression from PBMCs collected from control DTR WT mice and DTR Knock-In mice. (E) Flow cytometry analysis against DTR antibody on duplicate samples from mouse PBMCs.

FIG. 14. PPDP2 nanoparticles for delivery of rrsp mRNA to cancer cells. rrsp mRNA encapsulated in PPDP2 nanoparticles allows for cytoplasmic delivery and expression of full-length, functional RRSP protein. Active RRSP in the cytoplasm cleaves RAS, thus downregulating the RAS/RAF/MEK/ERK pathway, leading to cell death.

FIGS. 15A-15D. PPDP2 nanoparticles for deliver EGFP expression plasmid into pancreatic cancer cells. Representative fluorescent images of KPC (A) and PANC-1 (B) cells 24 hour after transfection of cells with 1 μg of pEGFP-N3 plasmid alone (top row), PPDP2 alone (second row) or pEGFP-N3 plasmid mixed with 2 μl Polyjet transfection reagent (SignaGen) (third row) or with PPDP2 (1:40 w/v %) (bottom row). gfp-mRNA with PPDP2 (third row) or gfp-mRNA with Lipofectamine MessengerMAX (Lipo) (bottom row). (C, D) Quantification of the fraction of GFP positive cells (green) from five imaged frames are shown as a histogram for KPC cells (C) and for PANC-1 cells (D). P values were calculated using a one-way ANOVA and Dunnett's multiple comparisons test, *p<0.05, **p<0.01, ***p<0.001.

FIGS. 16A-16B. PPDP2 nanoparticles for delivery of plasmid to cancer cells Western blot images of RAS in mouse pancreatic KPC 3813 (a) or human pancreatic PANC-1 (b) cells after 24 hour incubation untreated, treatment with PPDP2 alone, or treatment with indicated μg of RRSP-plasmid mixed with PPDP2 (1:40) ratio. Polyject tranfection reagent (SignaGen) used at 2 μl as control mixed with 2 μg RRSP-plasmid.

### DETAILED DESCRIPTION OF THE INVENTION

The advent of nucleic acids and plasmid delivery to cells using synthetic nanocarriers provides a key strategy for RAS/RAP1-specific endopeptidase (RRSP) to be expressed within cells resulting in degradation of RAS and loss of cell proliferation. Previous studies established that RRSP could be expressed in cells following transfection of plasmids with the primary genetic sequence for RRSP cloned under the control of eukaryotic expression promoter with the expression of RRSP resulted in cytotoxicity. Cell cytotoxicity following plasmid transfection was reported in U.S. Pat. No. 10,829,752B2. The possibility to express RRSP in cells when expressed from a nucleic acid was contemplated in U.S. Pat. No. 10,829,752B2. Lipid nanoparticles have been used to deliver an mRNA to colon cancer cells resulting in cell cytotoxicity, cleavage of RAS, and reduction of tumors. However, lipid nanoparticle transfection is typically toxic, and thus potentially challenging for cancer therapy that can require repeated injection. The lipid nanoparticles can also be costly to manufacture.

To achieve intracellular expression of RRSP, the inventors developed a strategy for delivery of nucleic acids into cancer cells using a PEG-b-PPS nanocarrier conjugated to a dendritic peptide to express RRSP in the cytoplasm for direct interaction with RAS resulting in its cleavage and degradation. Further, the inventors show that these nanoparticles can deliver RRSP encoding nucleic acids to cells in vitro and in vivo and RRSP is then expressed in the cell cytosol to cleave RAS, leading to cell death and xenograft tumor regression. Unexpectedly, the inventors found that a catalytically inactive form of RRSP also reduced tumor growth. Further, the inventors demonstrate that RRSP nucleic acids delivered through the nanoparticles, intratumorally, is non-toxic and not likely to result in resistance from emergence of a KRAS mutation, as all RAS isoforms are cleaved in the cell. Further, they show that systemic delivery of the RAS degrader systemic via a protein based delivery system is non-toxic indicating that this invention could be deployed systemically.

Accordingly, in a first aspect, a system for delivery of a nucleic acid encoding RAS/RAP1-specific endopeptidase (RRSP) to a cell is provided, the system comprising: a polymeric nanocarrier comprising a poly(ethylene glycol)-block-poly(propylene sulfide) copolymer (PEG-b-PPS) conjugated to a dendritic-specific branched cationic peptide (DP) (PEG-b-PPS-DP or PPDP); and the nucleic acid encoding RRSP.

PEG-b-PPS polymers conjugated with a dendritic peptide (DP) (PPDP) are assembled into stable nanostructures that encapsulate nucleic acids by mixing in aqueous buffer. This delivery system serves as an excellent platform for enhanced loading and delivery of genetic material for biomedical research and therapeutic applications with a unique capability to enhance intracellular delivery of nucleic acids to immune cells, which are notoriously difficult to transfect. Specifically, the nanocarrier platform described herein to deliver polynucleotides is nontoxic, and allows for efficient in vitro transfection of cells. This in vitro transfection of cells is allowed to take place in the presence of serum, and provides superior transfection as compared to the current lipofectamine standard for cell transfection, which is toxic and has been notorious for difficulty in transfecting immune cells. Lipofectamine requires special serum free medium and results in toxic effects and low cell viability after transfection.

The PEG-b-PPS may be conjugated with the DP via a linker. Linkers can also be called spacers and are known in the art and may comprise the amino acids glycine and or serine. The glycine and serine residues may repeat one or more times. Suitable linkers can include, but are not limited to, for example, 1) covalent bonds 2) ionic bonds or sensitive to 3) enzymatic degradation/proteolysis 4) pH 5) temperature 6) light 7) ultrasound 8) salt concentration 9) surfactants 10) oxidation 11) hydrolysis. Suitable methods of linking are known in the art. A linker group typically has two ends, wherein one of the ends comprises a substrate (DNA or RNA) attaching group and wherein the other of the ends comprises a polymer attaching group. The present invention is not limited to any particular linker group. Indeed, the use of a variety of linker groups is contemplated, including, but not limited to, alkyl, ether, polyether, alkyl amide groups or a combination of these groups. The present invention is not limited to the use of any particular substrate (DNA or RNA) attaching group or polymer attaching groups as they are known in the art. It is contemplated that a variety of polymer attaching groups may be used, including, but not limited to amine, hydroxyl, thiol, carboxylic acid, ester, amide, epoxide, isocyanate, and isothiocyanate groups. The linker may include a trityl moiety, an ester moiety, or a carboxylated dimethyl maleic acid (CDM) moiety. In exemplary embodiments, the linker is a disulfide bond or a pyridyl disulfide bond. However, any useful linker moiety can used.

Poly(ethylene glycol)-block-poly(propylene sulfide) copolymers (PEG-b-PPS or PEG-bl-PPS) copolymers can be prepared via known methods, for example those described in Allen, S. et al., Facile assembly and loading of theranostic polymersomes via multi-impingement flash nanoprecipitation J. Control. Release 2017. 262: p. 91-103 and in U.S. Pat. No. 10,633,493, each of which is incorporated herein by reference in its entirety with regard to the method of preparing the copolymers. The PEG-b-PPS polymers provide both hydrophobic moieties of PPS to stabilize the nanostructure and hydrophilic PEG corona to enhance cellular uptake and decrease toxicity. The integration of a bioreducible disulfide bond between PPS and DP improves gene delivery efficiency, due to the improved endosomal escape and cargo release in the reductive intracellular environment.

“Dendritic peptides,” “DP” or “branched cationic peptides” are peptides having a three-dimensional (3D) architecture with multiple functional groups. Dendritic peptides are branched oligocationic peptides that differ in the number and type (lysine, arginine, ornithine) of cationic amino acid. The DP has three generations and each unit is composed of positively charged arginine (R) for interaction with nucleic acids, histidine (H) with buffering capacity, lipophilic leucine (L) with membrane-binding ability to facilitate endosomal escape, and lysine (K) for functional unit branching.

These branched peptides show better encapsulation and transfection efficiency for gene delivery compared with linear peptides. A variety of parameters can affect the activity of dendritic peptides, including generations based on the layer of peptide branching, molecular weight, functional or branching units, and charge distribution. In one embodiment, the dendritic peptide conjugated to the PEG-b-PPS is {[(RHL)₂-KRHL]₂-KRHL}₂-KC-NH₂. The PEG-b-PPS may be conjugated to the DP via disulfide exchange, as described in Yi et al. iScience. 2022; 25(7): 104555, which is incorporated herein by reference in its entirety. The PEG-b-PPS to DP ratio may be from about 1:1 to about 1:1000, preferably about 1:1 to about 1:500. In some embodiments, the PEG-b-PPS to DP ratio is about 1:1, about 1:2, about 1:10, about 1:20, about 1:50, about 1:75, about 1:100, about 1:120, about 1:200, about 1:500, about 1:750, etc. including all ratios and ranges in between.

The term “nanocarrier,” “nanoparticle,” or “nanostructure” refers to a nanomaterial used as a transport module for another substance. For example, the nanocarriers disclosed herein may be used as a transport module for one or more therapeutic agents. PEG-b-PPS nanocarriers are non-inflammatory and are therefore advantageous as vehicles for immunomodulatory therapeutic agents, as the elicited responses are dependent solely on the transported therapeutic agent. PEG-b-PPS block copolymers can be prepared via known methods, e.g., those described in Allen, S. et al., Facile assembly and loading of theranostic polymersomes via multi-impingement flash nanoprecipitation, i.e., J. Control. Release 2017. 262: p. 91-103 and in U.S. Pat. No. 10,633,493, each of which is incorporated herein by reference in its entirety. For example, an appropriate methyl ether poly(ethylene glycol) with a mesylate leaving group can be reacted with thioacetic acid to form a protected PEG-thioacetate. Base activation of the thioacetate can result in the formation of a thiolate anion, which may be used as the initiator for ring opening polymerization of propylene sulfide. The reaction can be completed with the addition of an end-capping agent or functionalization agent. These block copolymers can be prepared with varying ratios of PEG and PPS by varying the degree of propylene sulfide polymerization.

Nanocarriers of the PEG-b-PPS can be prepared, for example, by flash-nanoprecipitation (FNP) or thin film rehydration. To make nanocarriers via FNP, polymer and any hydrophobic agents can be dissolved in one or more organic solvents, while any hydrophilic agents can be dissolved in an aqueous solution (e.g., a buffer such as phosphate-buffered saline). The two solutions can be loaded into separate syringes and impinged against each other into a reservoir using a confined impingement jets (CIJ) mixer. Multiple impingements can be used to extrude polymersomes. To make nanocarriers via thin film rehydration, polymer and any hydrophobic agents can be dissolved in one or more organic solvents, and the resulting solution can be dessicated. Then an aqueous solution (e.g., a buffer such as phosphate-buffered saline) can be added to the mixture can be shaken overnight, followed by extrusion (e.g., using a syringe filter). The polymersomes may be extruded using a 0.22 μm syringe filter. For both methods, unloaded agents can be removed either via exclusion column purification or dialysis. Nanocarriers can be characterized for size distribution via dynamic light scattering (DLS) and nanoparticle tracking analysis (NTA), and for morphology via cryogenic transmission electron microscopy (cryoTEM). Agent loading can be characterized via fluorescence and absorbance measurements.

The nanocarriers described herein can comprise PEGₘ-b-PPSₙ linked to the DP, wherein m and n are both integers each selected from 1-500, alternatively about 2-300, alternatively 10-250. Specific m and n can be selected to provide the specific ratio of PEG and PPS to provide the specific nanostructure desired (e.g., polymersome, bicontinous nanospheres, micelles, filomicelles etc. as described herein). Polymersomes are a class of artificial vesicle nanocarriers composed of amphiphilic synthetic block copolymers and having an aqueous core. Polymersome may be prepared via known methods, e.g., Du F. et al., Immunotheranostic Polymersomes Modularly Assembled from Tetrablock and Diblock Copolymers with Oxidation-Responsive Fluorescence. Cell. Mol. Bioeng. (2017). DOI: 10.1007/s12195-017-0486-7; Du F. et al., Sequential intracellular release of water-soluble cargos from Shell-crosslinked polymersomes. J Control Release.2018; 282:90-100; and Yi S., et al, Tailoring Nanostructure Morphology for Enhanced Targeting of Dendritic Cells in Atherosclerosis. ACS Nano. 2016; 10(12):11290-11303, each of which are incorporated herein by reference in their entirety. Micelles are a class of artificial vesicle nanocarriers having a hydrophobic/lipophilic core and a hydrophilic exterior. Micelle or filomicelle nanostructures have a spherical morphology and are typically smaller (e.g., less than 50 nm) than polymersomes and the hydrophobic core can be loaded with a nucleic acid. Micelles or filomicelles can be prepared via known methods, for example those described in Karabin, N. B., Allen, S., Kwon, H. et al. Sustained micellar delivery via inducible transitions in nanostructure morphology. Nat Commun 9, 624 (2018), which is incorporated herein by reference. Bicontinuous nanospheres (BCNs) are characterized by two continuous phases; (i) a cubic lattice of aqueous channels that traverse (ii) an extensive hydrophobic interior volume. Based on small angle X-ray scattering (SAXS) analysis, BCN have primitive type cubic internal organization (Im3m) as confirmed by Bragg peaks with relative spacing ratios at √2, √4, and √6. BCNs are the polymeric equivalent of lipid cubosomes and are lyotropic. BCNs can incorporate both hydrophobic and hydrophilic therapeutic agents. BCNs can be prepared via known methods, for examples those described in Allen, S. et al. Benchmarking bicontinuous nanospheres against polymersomes for in vivo biodistribution and dual intracellular delivery of lipophilic and water-soluble payloads. ACS Appl. Mater. Interfaces 2018, 10, 40, 33857-33866, which is incorporated herein by reference in its entirety regarding structure and characteristics for the continuous nanosphere structures.

The different PEG-b-PPS nanocarrier types can be prepared by varying the degree of propylene sulfide polymerization, oxidation or branching. For example, polymersomes typically have a PEG weight fraction of about 0.25 to about 0.45, micelles typically have a PEG weight fraction above 0.45, bicontinuous nanospheres typically have a PEG weight fraction below 0.25, and micelles and filomicelles typically have a PEG weight fraction of about 0.35 to about 0.45. The PEG-b-PPS nanocarrier of the present invention may have a PEG weight fraction of between about 0.01 and about 0.95 and any weight fractions and ranges in between, such as, for example between about 0.05 and about 0.9, between about 0.05 and about 0.50, between about 0.01 and about 0.25, between about 0.1 and about 0.2. In exemplary embodiments, the block copolymer is PEG₁₇PPS₈₀, in which the PEG weight fraction is 0.11 and the PPS weight fraction is 0.89. In exemplary embodiments, the DP-conjugated nanocarrier is PEG₁₇-PPS₈₀-ss-DP (PPDP2). One skilled in the art would be able to determine proper weight fractions and the weight fractions may vary from the examples provided herein but still be within the scope of the invention.

As used herein, the term “nucleic acid” or “polynucleotide” refers to deoxyribonucleic acid (DNA), ribonucleic acid (RNA) and DNA/RNA hybrids. Polynucleotides may be single-stranded or double-stranded. Nucleic acids include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA(+)), minus strand RNA (RNA(−)), synthetic RNA, genomic DNA (gDNA), PCR amplified DNA, complementary DNA (cDNA), synthetic DNA, or recombinant DNA. In some embodiments, the RRSP nucleic acid included in the system described herein is DNA encoding RRSP. The DNA may be in a plasmid or other vector, or a construct. In exemplary embodiments, the RRSP nucleic acid is a mRNA encoding RRSP. In embodiments, the RRSP nucleic acid includes any polynucleotide sequence encoding an RRSP protein, peptide or fragment thereof.

As used herein, the term “construct” refers to a recombinant polynucleotide, i.e., a polynucleotide that was formed artificially by combining at least two polynucleotide components from different sources (natural or synthetic). For example, the constructs described herein comprise the coding region of RRSP operably linked to a promoter that may be associated with another gene or may be synthetic. The RRSP may also be operably linked to its natural promoter. Constructs can be generated using conventional recombinant DNA methods.

Constructs may be part of a vector. As used herein, the term “vector” refers to a nucleic acid molecule capable of propagating another nucleic acid to which it is linked. The term includes the vector as a self-replicating nucleic acid structure as well as a vector incorporated into the genome of a host cell into which it has been introduced. Certain vectors are capable of directing the expression of nucleic acids to which they are operatively linked. Such vectors are referred to herein as “expression vectors” (or simply, “vectors”). Suitable vectors for use with the present invention comprise a promoter operably connected to a polynucleotide sequence encoding a protein or peptide of interest. The term vector encompasses “plasmids”, the most commonly used form of vector. Plasmids are circular double-stranded DNA loops into which additional DNA segments (e.g., those encoding peptides) may be ligated. In some embodiments, the vector is a mini-circle DNA (mcDNA) vector. Mini-circle DNA vectors are episomal DNA vectors that are produced as circular expression cassettes devoid of any bacterial plasmid DNA backbone. See, for example, System Biosciences, Mountain View CA, MN501A-1. Their smaller molecular size enables more efficient transfections and offers sustained expression over a period of weeks as compared to standard plasmid vectors that only work for a few days. The vectors may further a comprise heterologous nucleic acid backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide. Many commonly used expression vectors and plasmids contain non-human nucleic acid sequences, including, for example, CMV promoters.

The system described herein comprises a nanocarrier comprising PPDP and a nucleic acid encoding RRSP. RRSP, or Ras/Rap1-specific endopeptidases (RRSPs), formally known as DUF5, is a bacterial cytotoxic effector domain from the multifunctional-autoprocessing repeats-in-toxin (MARTX) toxin (Antic et al. 2015. Nat Commun 6, 7396). RRSP site-specifically cleaves RAS and its close homologue Repressor activator protein 1 (RAP1) between residues tyrosine-32 and aspartate-33 within the Switch I region, but not other closely related GTPases. Processing of RAS by RRSP disrupts its interaction with rapidly accelerated fibrosarcoma (RAF) kinases in the RAS-ERK (extracellular signal-regulated kinase) signaling axis. RRSP can cleave all three of the major RAS isoforms (H, N, and K), the most common oncogenic RAS mutations including G12C, G12D, G12V, G13D, and Q61R, and both GTP and GDP-bound RAS. Within cells, RRSP degradation of RAS leads to G1 cell cycle arrest that can progress to apoptosis, senescence, and loss of cell proliferation in more than 80% of all cells lines where it has been tested. The major limitation to use of RRSP as a therapeutic is that it is a 56-kiloDalton protein that alone cannot transit across the cell plasma membrane. The RRSP encoded by the nucleic acid includes the full protein, a homolog thereof, or an active portion thereof comprising the C2A subdomain and/or the C2B subdomain. As previously described in U.S. Pat. No. 10,829,752B2, the C2B and C2B in combination with C2A subdomains exhibit protease activity.

The nucleic acid encoding RRSP may comprise an mRNA sequence having at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 2 or SEQ ID NO: 4. The RRSP mRNA of SEQ ID NO: 2/4 is derived from Vibrio vulnificus. RNA encoding RRSP homologs from other organisms may be used, such as, for example, Vibrio ordalii, Vibrio cholerae, Vibrio splendidus, Moritella dasanensis, Aeromonas salmonicida, Aeromonas hydrophila, Photorhabdus temperate, Xenorhabdus nematophila, Photorhabdus luminescences, Photorhabdus asymbiotica, Yersinia kristensenii, and Pasteurella multocida. RRSP homologs are further characterized and described in U.S. Pat. No. 10,829,752B2, which is incorporated herein by reference.

The RRSP protein may be catalytically inactive. The catalytically inactive RRSP may be encoded by a nucleic acid comprising a sequence having at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 3.

The RRSP mRNA derived from Vibrio vulnificus encodes a polypeptide having the sequence of SEQ ID NO: 8. Therefore, the RRSP protein may comprise a sequence having at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to

(SEQ ID NO: 8)
QELKERAKVFAKPIGASYQGILDQLDLVHQAKGRDQIAASFELNKKINDY
IAEHPTSGRNQALTQLKEQVTSALFIGKMQVAQAGIDAIAQTRPELAARI
FMVAIEEANGKHVGLTDMMVRWANEDPYLAPKHGYKGETPSDLGFDAKYH
VDLGEHYADFKQWLETSQSNGLLSKATLDESTKTVHLGYSYQELQDLTGA
ESVQMAFYFLKEAAKKADPISGDSAEMILLKKFADQSYLSQLDSDRMDQI
EGIYRSSHETDIDAWDRRYSGTGYDELTNKLASATGVDEQLAVLLDDRKG
LLIGEVHGSDVNGLRFVNEQMDALKKQGVTVIGLEHLRSDLAQPLIDRYL
ATGVMSSELSAMLKTKHLDVTLFENARANGMRIVALDANSSARPNVQGTE
HGLMYRAGAANNIAVEVLQNLPDGEKFVAIYGKAHLQSHKGIEGFVPGIT
HRLDLPALKVSDSNQFTVEQDDVSLRVVYDDVANKPKITFKGSL.

The nucleic acid encoding RRSP may comprise a DNA sequence having at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 7. The DNA may be expressed in a construct comprising a heterologous promoter. The RRSP DNA may be expressed in a plasmid. In embodiments, the DNA sequence comprises at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 6.

The terms “percent identity” and “% identity” refer to the percentage of residue matches between at least two polynucleotide or polypeptide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid or peptide sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases and “blastp,” that is used to align a known amino acid sequence with other amino acids sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide or polypeptide sequence, for example, as defined by a particular SEQ ID number, or may be measured over a shorter length, for example, over the length of a fragment taken from a larger, defined polynucleotide or polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, Figures or Sequence Listing, may be used to describe a length over which percentage identity may be measured.

Structural similarity of a synthetic designed protein that has a three-dimensional structure that aligns with the three-dimensional determined crystal structure of RRSP (RCSB Protein Data Bank identification numbers 5W6L (DOI: 10.1126/scisignal.aat8335) 6A7H (DOI: 10.1074/jbc.RA118.004857) and 6A8J (DOI: 10.1074/jbc.RA118.004857) can also be based on conservation of the protein fold. The comparison can be based on structural comparison tools integrated into common structure analysis software suites and servers such as PyMol, UCSF ChimeraX, DALI, and HHPRED.

The terms “peptide,” “polypeptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The terms polypeptide, peptide, and protein are also inclusive of modifications including, but not limited to, glycosylation, lipid attachment, sulfation, carboxylation, hydroxylation, ADP-ribosylation, and addition of other complex polysaccharides. The terms “residue” or “amino acid residue” or “amino acid” are used interchangeably to refer to an amino acid that is incorporated into a peptide, protein, or polypeptide. The amino acid may be a naturally occurring amino acid and, unless otherwise limited, may encompass non-natural analogues of natural amino acids that can function in a similar manner as naturally occurring amino acids.

“Homology” refers to sequence similarity or, interchangeably, sequence identity, between two or more polypeptide sequences. Homology, sequence similarity, and percentage sequence identity may be determined using methods in the art and described herein.

A “variant,” “mutant,” or “derivative” of a particular polypeptide sequence may be defined as a polypeptide sequence having at least 20% sequence identity, but less than 100% identity, to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide. In particular embodiments, the term “gene” refers to a cDNA. cDNA are polynucleotides the encode for a protein and do not contain introns and can be artificially produced.

The nucleic acid may comprise a cell-specific promoter. The term “promoter” refers to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The cell-specific promoter may be a cancer-specific promoter. Examples of cancer-specific promoters include, but are not limited to, A33, glycoprotein A33; AFP, alpha fetal protein; Bmi-1, B cell-specific Moloney leukemia virus insertion site 1; CCKAR, cholecystokinin type A receptor; CEA, carcinoma embryonic antigen; COX-2, cyclooxygenase-2; FGF, fibroblast growth factor; HER-2, human epidermal growth factor receptor 2; hTERT, human telomerase reverse transcriptase; KDR, kinase domain insert containing receptor; PB, probasin; PSA, prostate-specific antigen; Rad51, rad51 recombinase; TTF-1, thyroid transcription factor-1; uPAR, urokinase-type plasminogen activator receptor.

The system described herein may be prepared using any standard mixing or nanoparticle fabrication method, including thin film hydroation and nano-precipitation, as well as scalable methods such as flash nano precipitation and microfluidics. In exemplary embodiments, the system is prepare by mixing PPDP with the nucleic acid under physiologic conditions, or under mildly acidic conditions such as, for example, in 25 mM sodium acetate buffer.

Once assembled, the delivery system may comprise any suitable mass ratio of PPDP to nucleic acid necessary to achieve the desired effect. For example, the delivery system may comprise a mass ratio (w/w) of PPDP:nucleic acid of between about 0.1:1 to about 1000:1, about 15:1 to 120:1, and any ratios and ranges in between. For example, the mass ratio may be 5:1, 10:1, 15:1, 30:1, 50:1, 75:1, 100:1, 115:1, 120:1, or 130:1. In preferred embodiments, the mass ratio of PPDP:DNA is 60:1. In exemplary embodiments, the mass ratio of PPDP:mRNA is 40:1.

The nanocarriers disclosed herein may also be incorporated into pharmaceutical compositions. The nanocarriers or pharmaceutical compositions comprising the nanocarriers may be used in methods of gene therapy in a subject in need thereof. The pharmaceutical compositions may further comprise one or more pharmaceutically acceptable excipients. The pharmaceutically acceptable excipients will be dependent on the mode of administration to be used. Suitable modes of administration include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseous, periocular, intratumoral, intracerebral, and intracerebroventricular administration. In some embodiments, the disclosed pharmaceutical compositions are administered parenterally. In some embodiments, parenteral administration is by intrathecal administration, intracerebroventricular administration, or intraparenchymal administration. In embodiments, the disclosed pharmaceutical compositions are administered subcutaneously. In embodiments, the disclosed pharmaceutical compositions are administered intravenously.

The nanocarrier may further comprise at least one therapeutic agent. The therapeutic agent may be any suitable therapeutic agent to achieve the desired therapeutic effect. The therapeutic agent may be hydrophilic or hydrophobic. In some embodiments, a nanocarrier comprising the at least one therapeutic agent is able to achieve the same immunomodulatory effects at a lower therapeutically effective dose compared the therapeutically effective dose required for free therapeutic agent (i.e., the therapeutic agent in the absence of the nanocarrier), therefore allowing therapeutic efficacy with minimized side effects in the subject. In other embodiments, the nanocarriers may enable a high dose of the therapeutic agent to be used safely without negative side effects typically associated with the same dose of the therapeutic agent in the absence of the nanocarrier. The disclosed nanocarriers may therefore improve the quality of life for patients, such as patients requiring immunosuppression for organ transplantation or inflammatory diseases, as the intended effect of the therapy will be achieved with reduced side effects. The nanocarrier may comprise any suitable number of therapeutic agents to achieve the desired effect.

The nanocarrier may further comprise a targeting ligand displayed on a surface of the nanocarrier. The targeting ligand may target any desired cell type. In some embodiments, the targeting ligand may selectively target cancer cells or other nonphagocytic cells. The targeting ligand may comprise an antibody, antibody fragment, an aptamer, or a peptide.

### Methods

In a second aspect, the present disclosure also provides methods of delivering an RRSP-encoding nucleic acid to a cell, the method comprising contacting or contacting the cell with the nanocarrier system disclosed herein. The nucleic acid may be DNA or RNA. The cell may be a mammalian cell. The cell may be a non-phagocytic cell. The cell may be a cancer cell. In particular, the cell may be a cancer cell with a mutation is the RAS gene. Ras genes encode proteins that can cause cancer or become oncogenic when mutated. Ras proteins are GTPases which act as molecular switches in the cell, regulating signaling pathways and other interactions. Ras signaling is an important intracellular signaling pathway that plays a role in cellular proliferation and differentiation, survival, and gene expression. Ras oncoprotein has also been implicated in the development of cancer by either having increased intensity or prolonged signaling mechanism. The cancer may include, but is not limited to lung cancer, colon cancer, skin cancer, pancreatic cancer, or breast cancer.

“Contacting” as used herein, refers to contacting a sample directly or indirectly in vitro, ex vivo, or in vivo. Contacting a cell may include addition of the system to a sample including the cell. Further, contacting a cell includes adding the system to a cell culture. The contacting may comprise culturing the cells in culture medium comprising the nanocarrier system and serum. The cell should be contacted for a sufficient time and under sufficient conditions to allow the cell to uptake the nanocarrier system and deliver the nucleic acid to the cell. The cell may essentially be transfected by the system by contacting the cell for a sufficient time and under sufficient conditions to allow the system to the nucleus of the cell. The polynucleotide is capable of expressing RRSP protein when it is transcribed and translated within the nucleus of the cell. Contacting also encompasses administration of the system to a solution, tissue, or subject.

The nanocarrier systems may be contacted to the cells at an amount that is non-toxic to the cells. The nanocarrier system results in a high delivery efficiency of the polynucleotide, specifically when compared to methods of the art such as lipofectamine. The term “non-toxic” refers to the ability of the nanocarrier system to not cause apoptosis or cell death when incubated or put into contact with the host cells. Suitably, non-toxic system refers to the ability to retain a majority of the cells being contacted with the nanocarrier system viable and able to incorporate the polynucleotide. Suitably, the term non-toxic refers to not causing a statistically significant decrease in cell viability as assessed by live/dead or metabolic assays (e.g., but not limited to MTT assay or CellTiterGlo) or other proliferation assays including, but not limited to, a crystal violet cell staining assay to quantify cell expansion after reseeding). Non-toxic may also refer to the ability of the system described herein to not cause unwanted, adverse off-target or side effects following administration to a cell or subject.

In a third aspect, the present disclosure also provides methods of treating a cancer in a subject by administering a therapeutically effective amount of the systems or pharmaceutical compositions thereof described herein. In embodiments, the cancer is a lung cancer, a colon cancer, a pancreatic cancer, a skin cancer, or a breast cancer. As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process. As used herein, the term “administering” refers to dispensing, delivering or applying the substance to the subject. In exemplary embodiments, the system is delivered intratumorally. In other preferred embodiments, the system is administered systemically.

The term “subject” or “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Preferably, the subject is a human. In some embodiments, the subject is a mammal in need of gene therapy. The term “subject” does not denote a particular age or sex. In one specific embodiment, a subject is a mammal, preferably a human.

The disclosed pharmaceutical compositions herein can be administered as the sole active agent or in combination with other pharmaceutical agents such as other agents used in the treatment of genetic disease in a subject. The amount of the disclosed nanocarriers or pharmaceutical compositions comprising the same to be administered is dependent on a variety of factors, including the severity of the condition, the age, sex, and weight of the subject, the frequency of administration, the duration of treatment, and the like. The disclosed nanocarriers or pharmaceutical compositions may be administered at any suitable dosage, frequency, and for any suitable duration necessary to achieve the desired therapeutic effect, i.e., to treat genetic disease. The disclosed nanocarriers or pharmaceutical compositions may be administered once per day, twice per day, or multiple times per day. The disclosed nanocarrier or pharmaceutical compositions may be administered daily, every other day, every three days, every four days, every five days, every six days, once per week, once every two weeks, or less than once every two weeks. The nanocarriers or pharmaceutical compositions may be administered for any suitable duration to achieve the desired therapeutic effect. For example, the nanocarriers or pharmaceutical compositions may be administered to the subject for one day, two days, three days, four days, five days, six days, seven days, eight days, nine days, ten days, eleven days, twelve days, thirteen days, two weeks, one month, two months, three months, six months, 1 year, or more than 1 year.

Any suitable dose of the disclosed nanocarriers or pharmaceutical compositions comprising the same may be used. Suitable doses will depend on the therapeutic agent, intended therapeutic effect, body weight of the individual, age of the individual, and the like. In general, suitable dosages of the disclosed nanocarriers or pharmaceutical compositions comprising the same may range from about 0.025 mg nanocarrier/kg body weight to 200 mg nanocarrier/kg body weight. For example, suitable dosages may be about 0.025 mg/kg, or 0.03 mg/kg, or 0.05 mg/kg, or 0.10 mg/kg, or 0.15 mg/kg, or 0.30 mg/kg, to 0.5 mg/kg, or 0.75 mg/kg, or 1.0 mg/kg, or 1.25 mg/kg, or 1.5 mg/kg, or 1.75 mg/kg, or 2.0 mg/kg. In some embodiments, the suitable doses may be 1 mg nanocarrier/kg body weight, or 3 mg/kg, or 5 mg/kg, or 10 mg/kg, or 25 mg/kg, or 50 mg/kg, or 75 mg/kg, or 100 mg/kg, or 125 mg/kg, or 150 mg/kg, or 175 mg/kg, or 200 mg/kg. In some embodiments, the pharmaceutical composition or nanocarrier may be administered intravenously.

### Miscellaneous

The present invention has been described in terms of one or more preferred and exemplary embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or Figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The following Examples are illustrative and should not be interpreted to limit the scope of the claimed subject matter.

### Example 1—(PPDP2) Nanoparticles for Delivery of an RRSP mRNA to Cells

In the following example, the inventors describe a novel targeted cancer therapy that employs mRNA-loaded nanoparticles to interrupt the RAS signaling system within cancer cells. The nanoparticles are composed of the polymer poly(ethylene glycol)-b-poly(propylene sulfide) (PEG-b-PPS) conjugated to a dendritic cationic peptide (DP) (26), and are demonstrated to deliver RRSP mRNA into cancer cell cytoplasm for direct interaction with RAS resulting in its cleavage and degradation. The optimally sized PEG₁₇-PPS₈₀-ss-DP (PPDP2) molecule undergoes pH-dependent disorder-to-order transitions to adopt a unique helical conformation under acidic conditions that promotes the cytoplasmic release of diverse payloads intracellularly.

PPDP2 Nanoparticles Deliver Gfp-mRNA into Pancreatic Cancer Cells and mCherry-mRNA into Pancreatic Cancer Xenografts

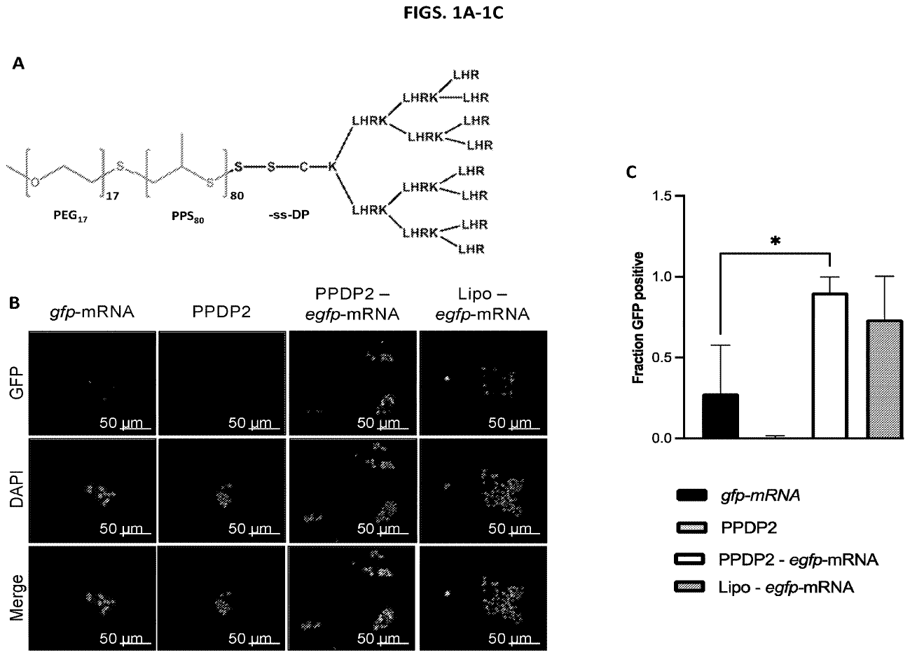

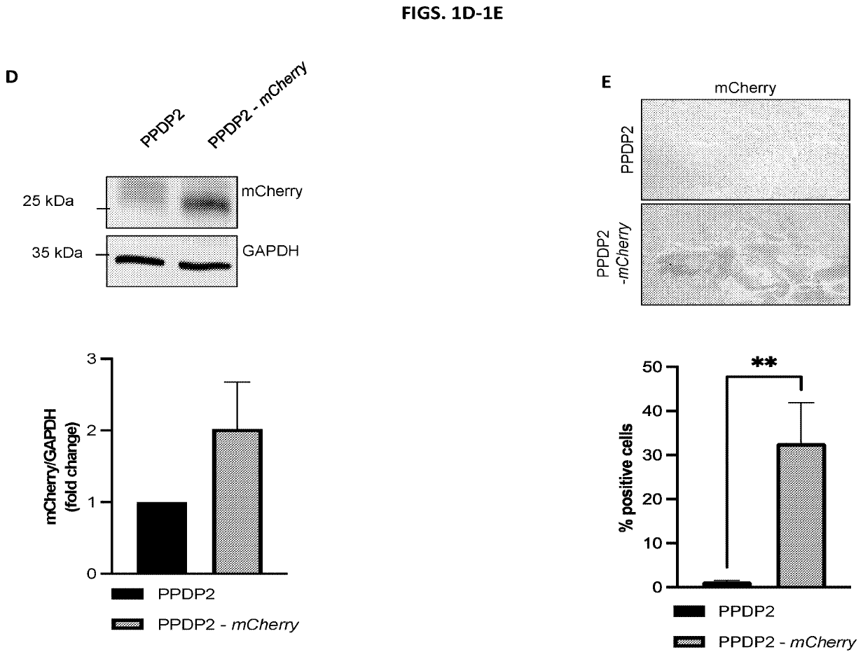

PPDP2 has been demonstrated to deliver payloads to immune cells but has not previously been tested against nonphagocytic cancer cells. The first objective was to test whether PPDP2 nanoparticles (FIG. 1A) can deliver mRNA into cancer cells. In human pancreatic PANC-1 cells, PPDP2 nanoparticles successfully delivered gfp-mRNA [1 μg] to cancer cells resulting in green fluorescent protein (GFP)-positive cells with about 80% of cells transfected (FIG. 1B). Expression of GFP was 2.5-fold higher than gfp-mRNA alone and comparable to the comparison transfection reagent lipofectamine.

In addition, to determine in vivo expression and localization of the cargo within our PPDP2 nanoparticles, a pilot experiment with mCherry mRNA delivered by PPDP2 through intratumoral injection was performed. Here, after 4 weeks of treatment, 3× per week, the tumors showed high expression of mCherry, through western blotting and immunohistochemical (IHC) staining (FIGS. 1C-1D).

### PPDP2-Rrsp Reduces Levels of RAS and Impacts Cell Proliferation in Pancreatic Cancer Cells

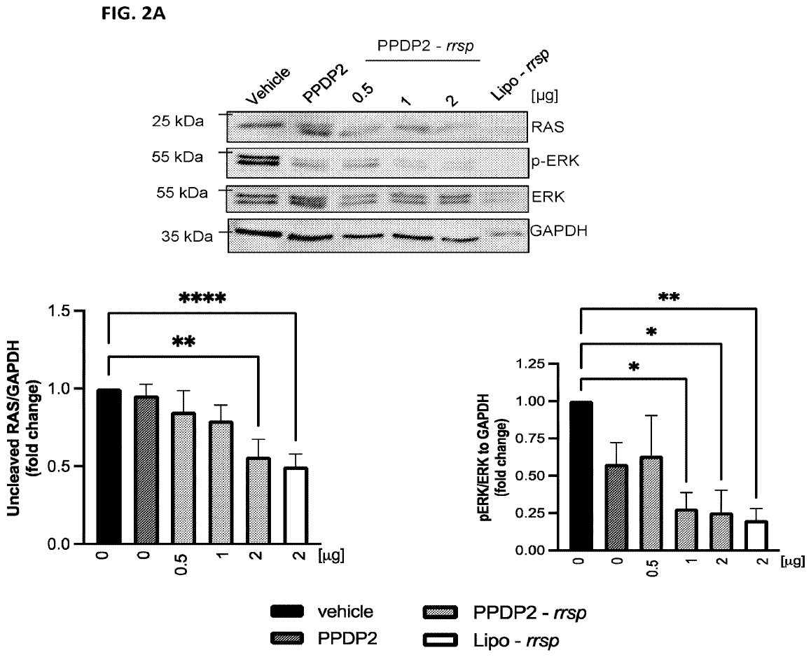

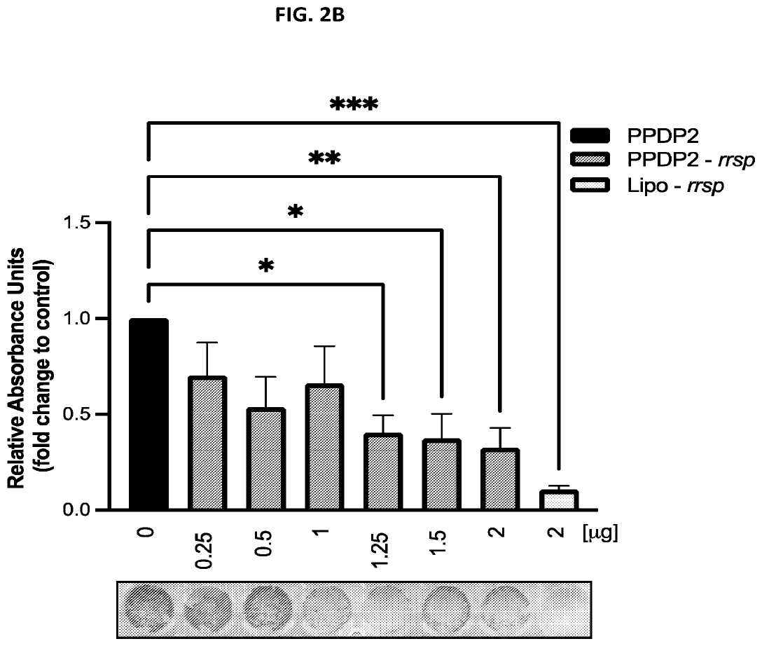

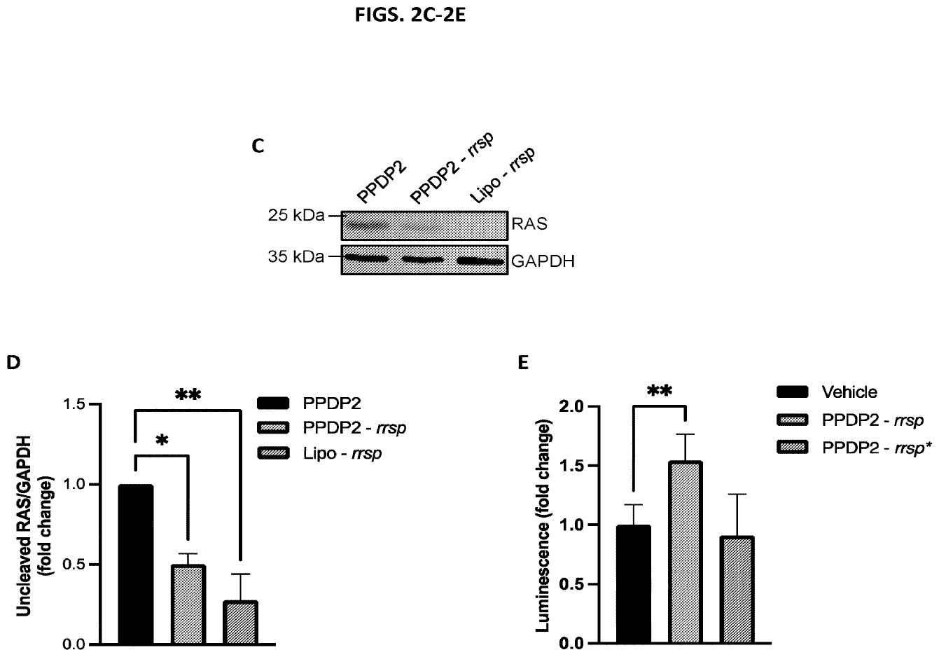

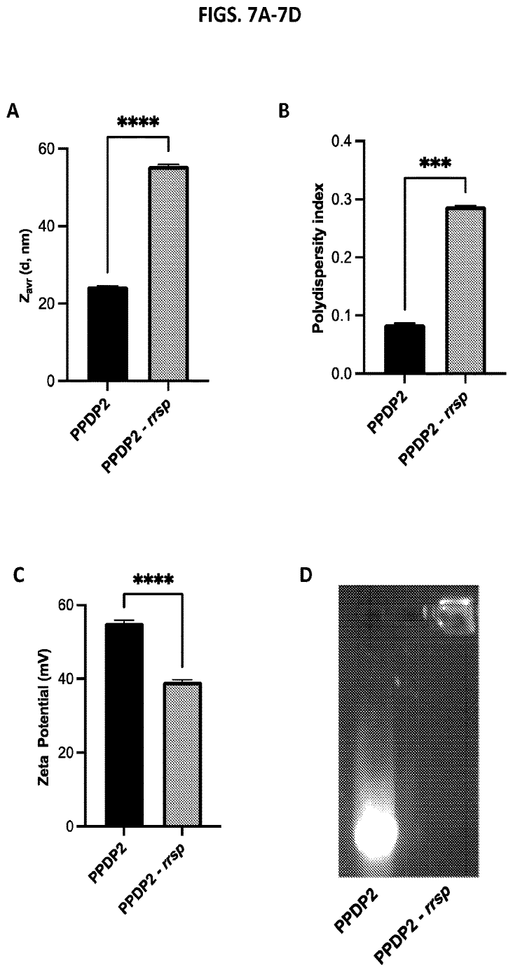

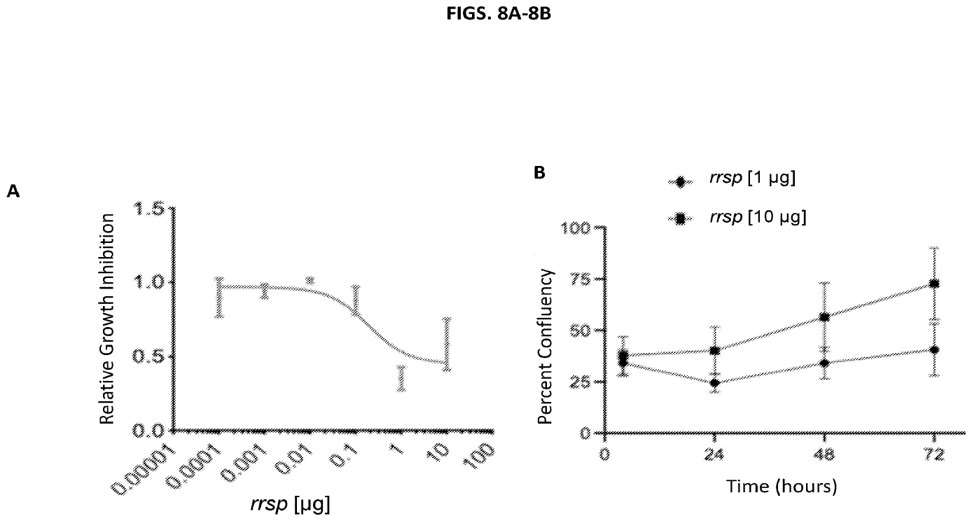

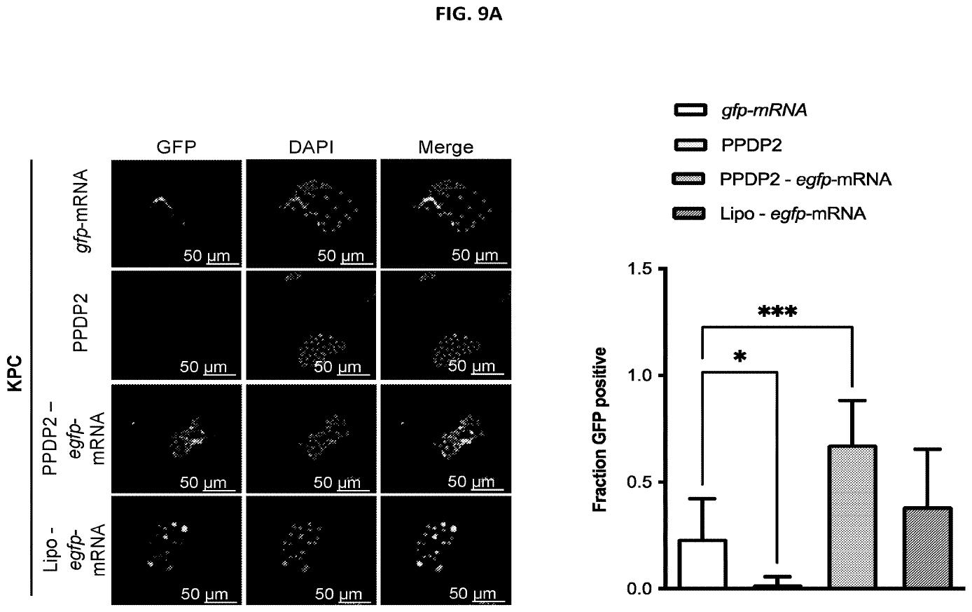

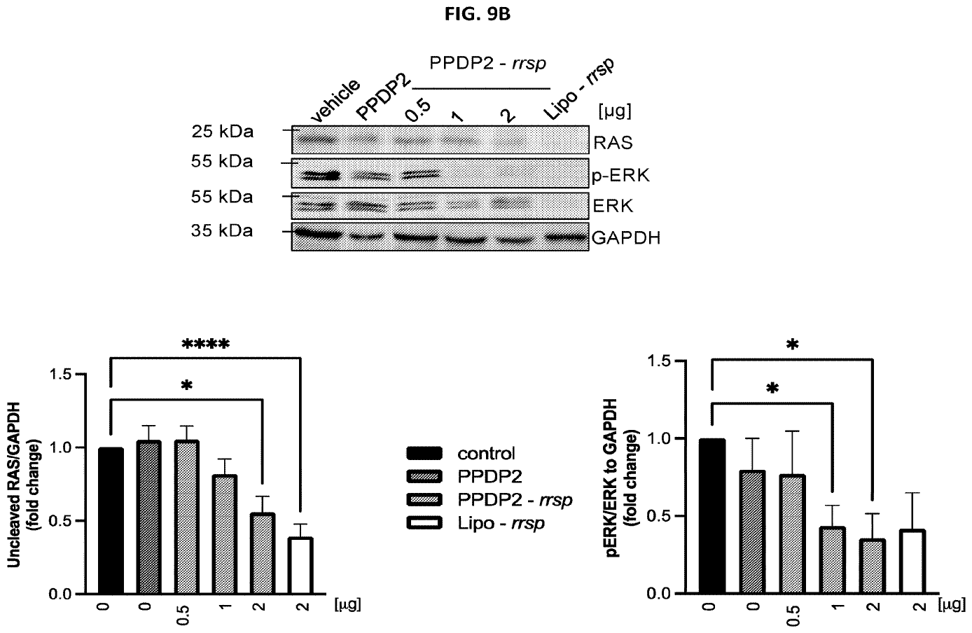

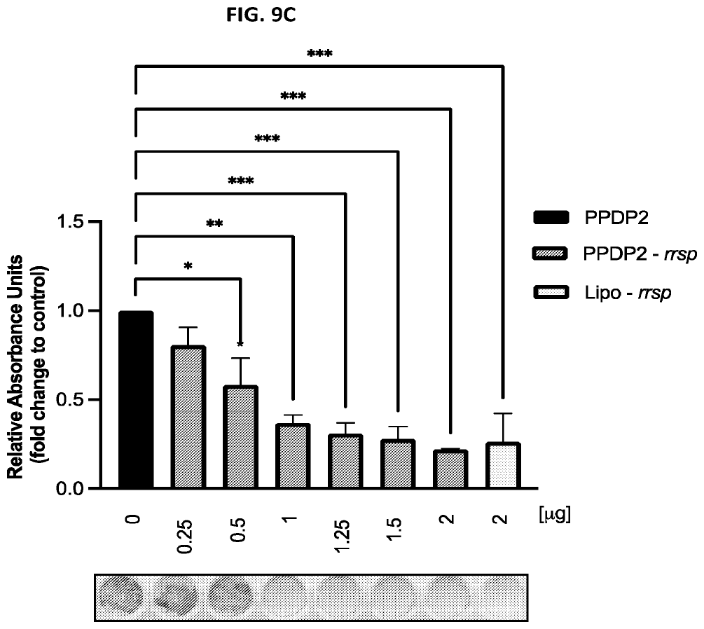

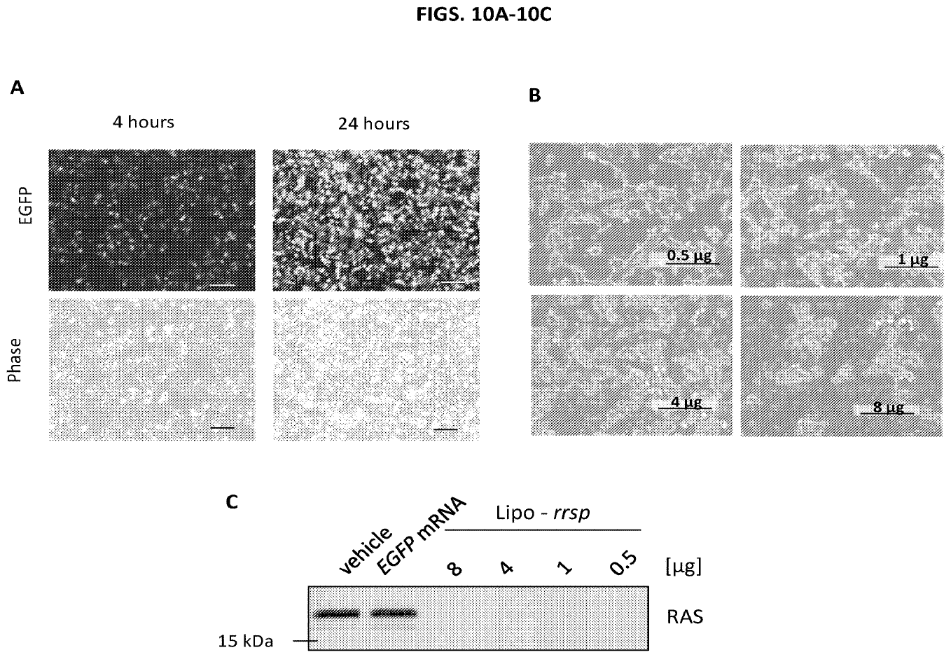

Next, we sought to determine whether if mRNA for expression of RRSP could be delivered within cells using PPDP2 synthetic nanoparticles. After validating loading of rrsp mRNA into PPDP2 nanoparticles (FIG. 7), we treated PANC-1 cells with PPDP2 nanoparticles loaded with varying concentrations of rrsp-mRNA (PPDP2-rrsp). The stability of RAS was monitored with a pan-RAS antibody that recognizes the conserved Switch 1 and thus only intact RAS is detected by western blotting. In treated cells, we found a 40% reduction in total levels of intact RAS (FIG. 2A). The phosphorylation of ERK was also significantly reduced indicative of RRSP inhibiting the signaling pathway downstream of RAS. PPDP2-rrsp at even 1.25 μg in PANC-1 cells showed significant reduction (over 60%) in cell proliferation measured by crystal violet staining (FIG. 2B). We also tested mouse pancreatic KPC cells, finding similar results (FIG. 8A). These data support that PPDP2 nanoparticles can be used for delivery of mRNA to nonphagocytic cancer cells. In addition, there was an increase in the activity of caspases 3 and 7 indicating an increase in cell death upon treatment with 5.5 μg rrsp mRNA in PANC-1 cells (FIGS. 2C-2D). These experiments were replicated in mouse KPC cells (FIGS. 9B-9C) and in human HCT-116 colorectal carcinoma cells (FIG. 10). After 24 h transfection, RAS levels were reduced with only 0.5 μg of rrsp delivered via Lipofectamine MessengerMAX (FIG. 9) and even 0.5 μg caused changes to cell morphology and reduced confluency was observed at 4 and 8 μg (FIG. 10B). Overall, rrsp mRNA delivery with PPDP2 causes loss of RAS and downstream p-ERK expression as well as reduced cell proliferation and viability indicating RAS is cleaved and is detrimental to the survival of cancer cells.

### PPDP2-Rrsp Reduces Pancreatic Tumor Growth

After validation of mRNA delivery and expression in vivo with PPDP2 nanoparticles, we next determined whether rrsp mRNA delivered with PPDP2 nanoparticles could lead to RRSP protein expression and cleavage of RAS in vivo. Based on our in vitro experiments of 2 μg rrsp mRNA causing loss of RAS and p-ERK expression, we increased the in vitro dose 2.5× to 5.5 μg rrsp mRNA, equivalent to 0.25 mg/kg, a dose similar to 10 μM in vitro, that significantly reduced RAS levels and increased caspase activation in PANC-1 cells (FIGS. 2C-2E).

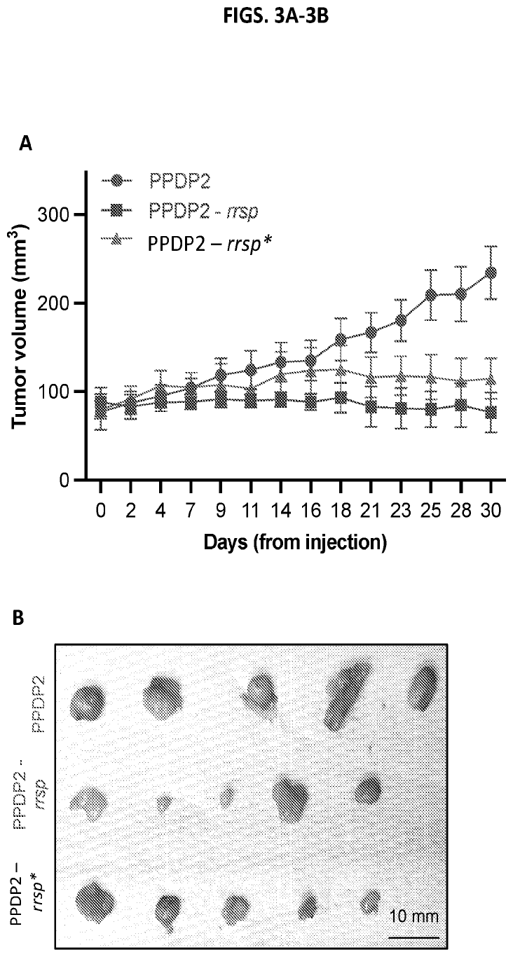

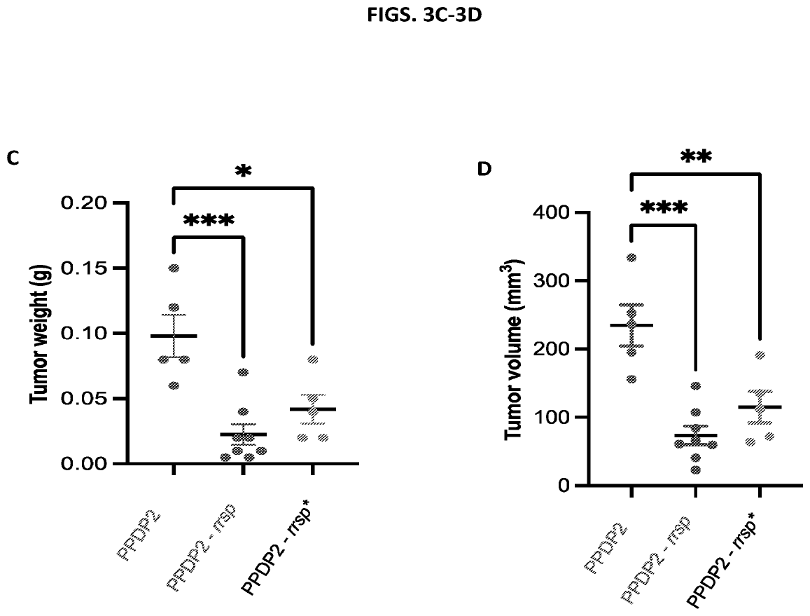

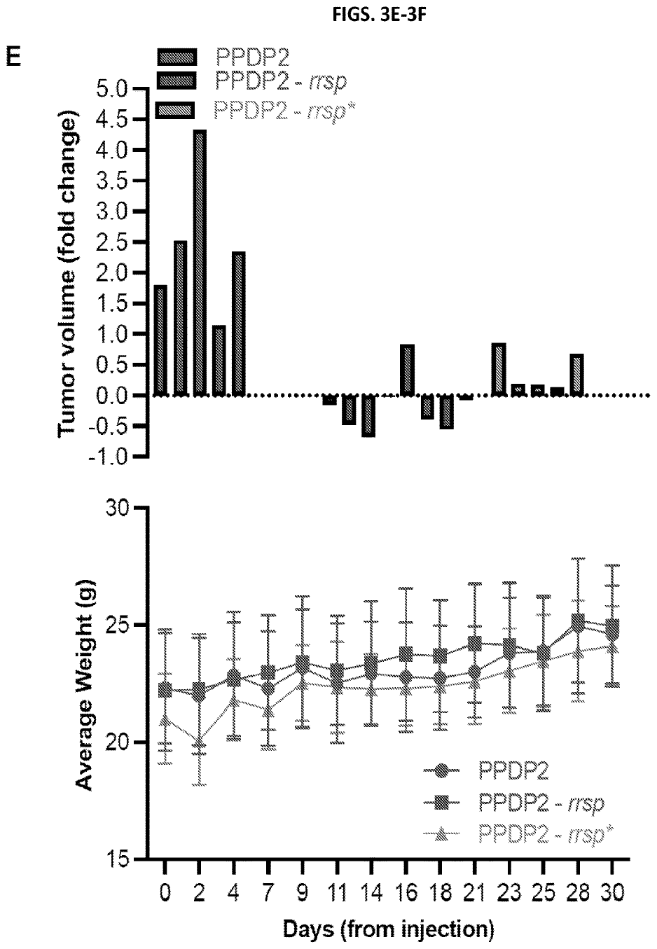

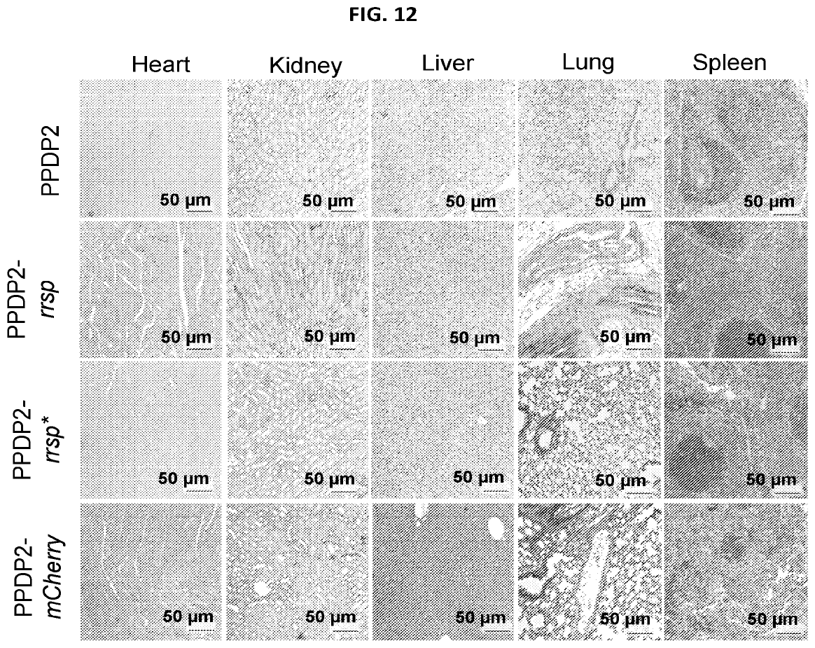

Here, we compared PPDP2 nanoparticle treatment alone with the PPDP2-rrsp treatment, at the 0.25 mg/kg dose 3×/week for 4 weeks through intratumoral injection. As a negative control, we delivered mRNA for a catalytically inactive form of RRSP (rrsp*). There was a significant reduction in tumor size for the PPDP2-rrsp treatment group with tumors reduced by 70-80% (FIGS. 3A-3E). Unexpectedly, the catalytically inactive form of RRSP (rrsp*) reduced tumor growth by 50-60%. This may be due to RRSP* retaining the ability to bind to RAS (22) and by preventing access of RAF and/or GTP to the Switch 1 region. In addition, mouse weight remained stable throughout the experiment (FIG. 3F), and tissue architecture within the major organs was unchanged (FIG. 12) indicating that RAS inhibition is non-toxic. Overall, we found that PPDP2 nanoparticles are capable of delivering both mCherry and rrsp/rrsp*mRNA in vivo. In addition, delivery of rrsp/rrsp*mRNA caused significant tumor regression without off-target toxicities suggesting the promise of this combination therapeutic and delivery system for pancreatic cancer treatment.

PPDP2-Rrsp Reduces Proliferation, RAS and p-ERK Expression in Mouse Pancreatic Tumors

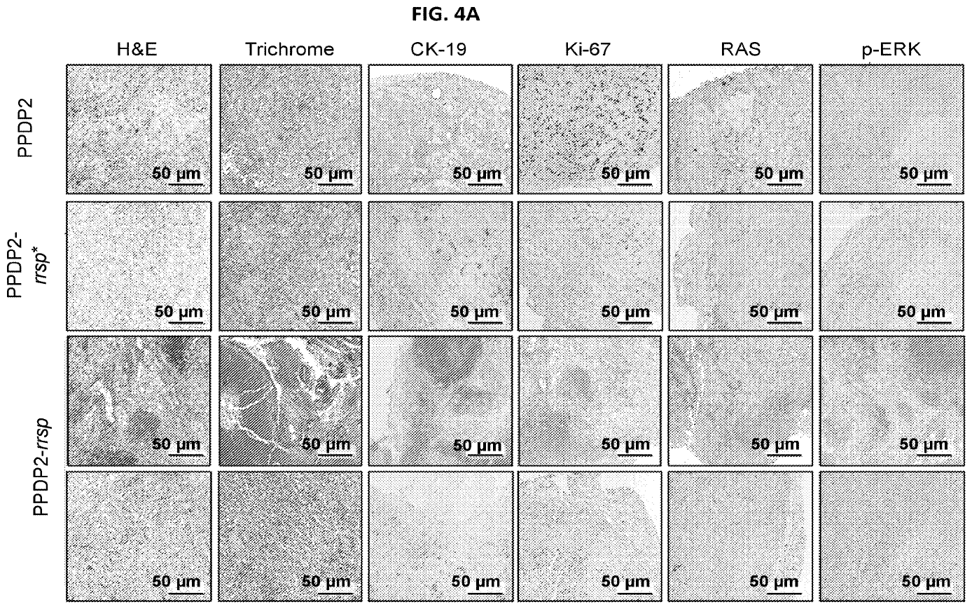

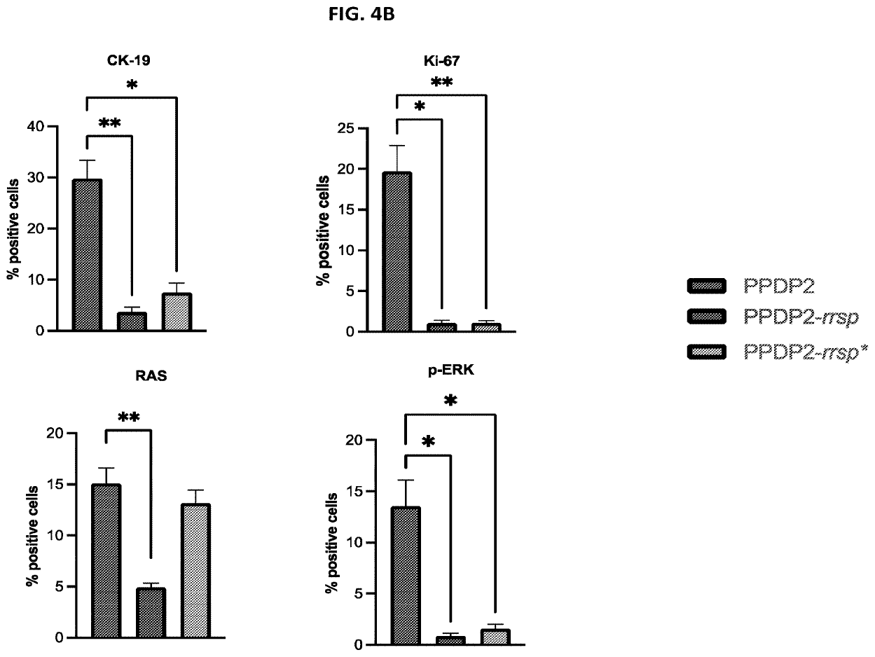

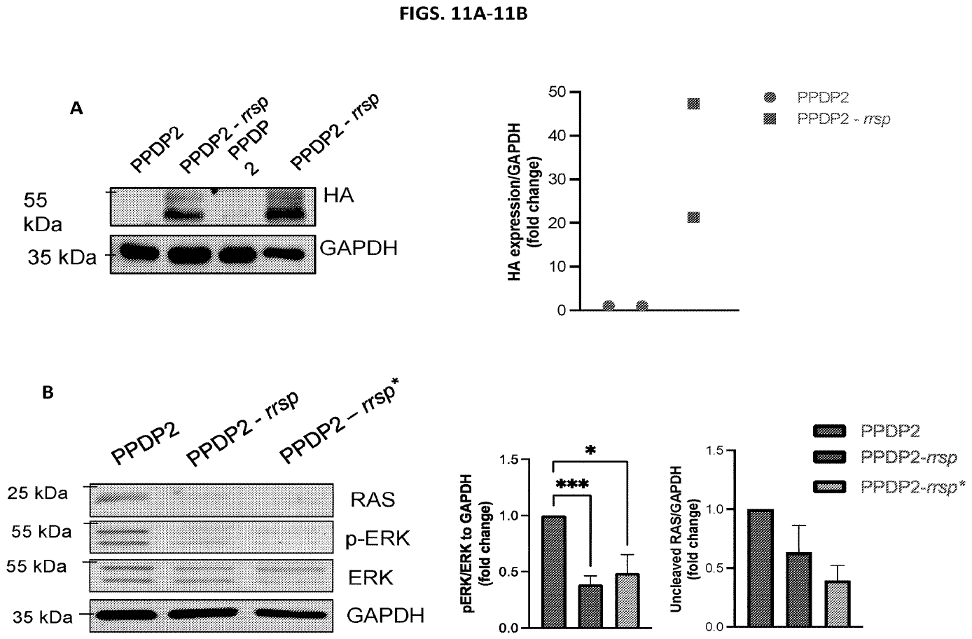

To validate whether PPDP2-rrsp targeted RAS in vivo, we performed western blotting on mouse tumor samples. We tested a hemagglutinin (HA) antibody to detect our HA-tagged RRSP and found measurable protein levels (FIG. 11A). In addition, we observed reduced levels of RAS leading to a statistically significant loss of p-ERK for both rrsp mRNA and rrsp*mRNA treated tumors (FIG. 11B). This result further supports that highly expressed catalytically inactive RRSP* may be functioning by binding to RAS and thus inhibiting recruitment and activation of RAF and downstream effects on ERK (FIG. 10B). We also performed IHC staining for cytokeratin-19, a marker of tumor progression, Ki-67, a marker of cell proliferation, and both RAS and p-ERK. We saw a statistically significant reduction in all four markers in PPDP2-rrsp treated mice. Although IHC staining did not show significant loss of RAS with rrsp* treatment, p-ERK levels were reduced indicating again that highly expressed RRSP* is likely binding to RAS which may be sufficient to inhibit its activation and downstream ERK expression (FIGS. 4A-4B). Overall, our data show that both rrsp and rrsp*mRNA delivered through PPDP2 nanoparticles produced RRSP protein that can significantly inhibit RAS signaling, demonstrating the mechanism of tumor regression.

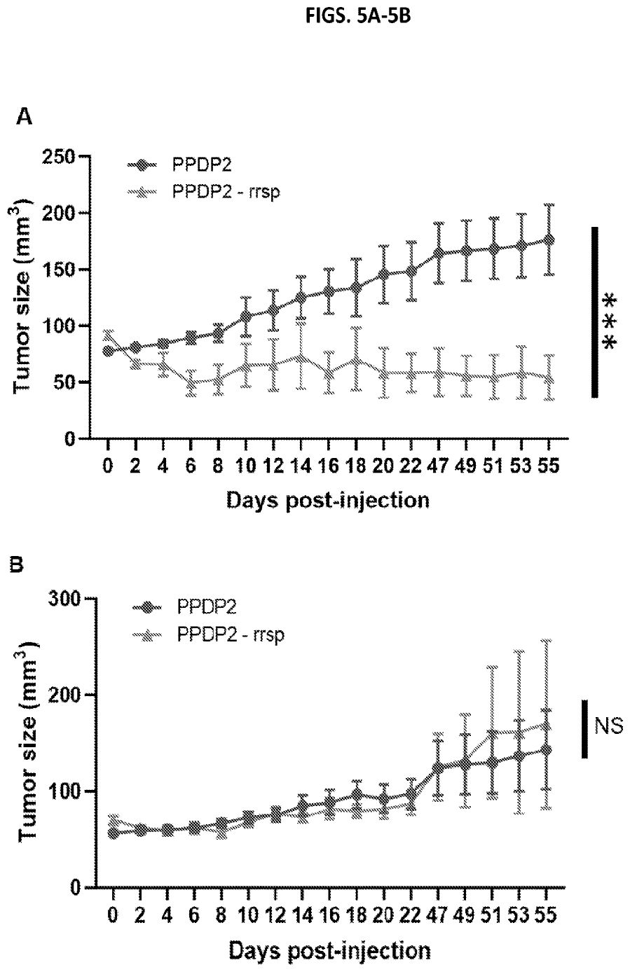

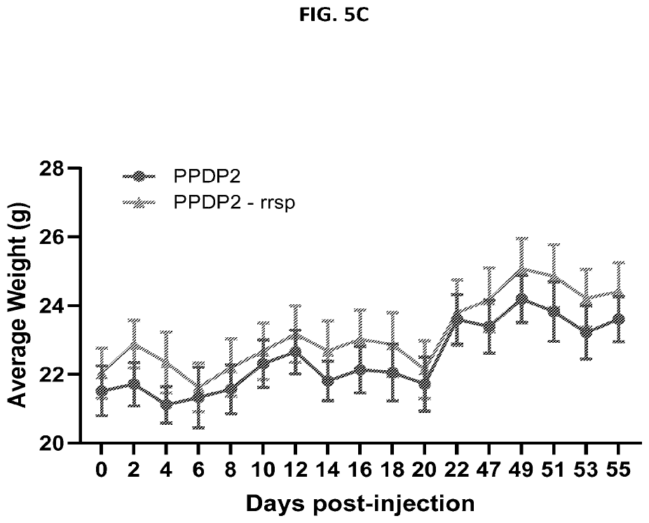

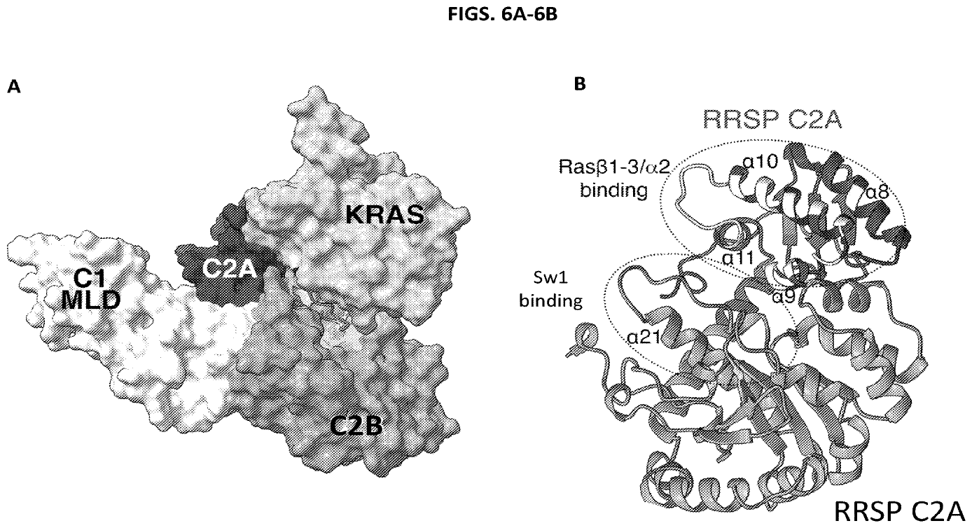

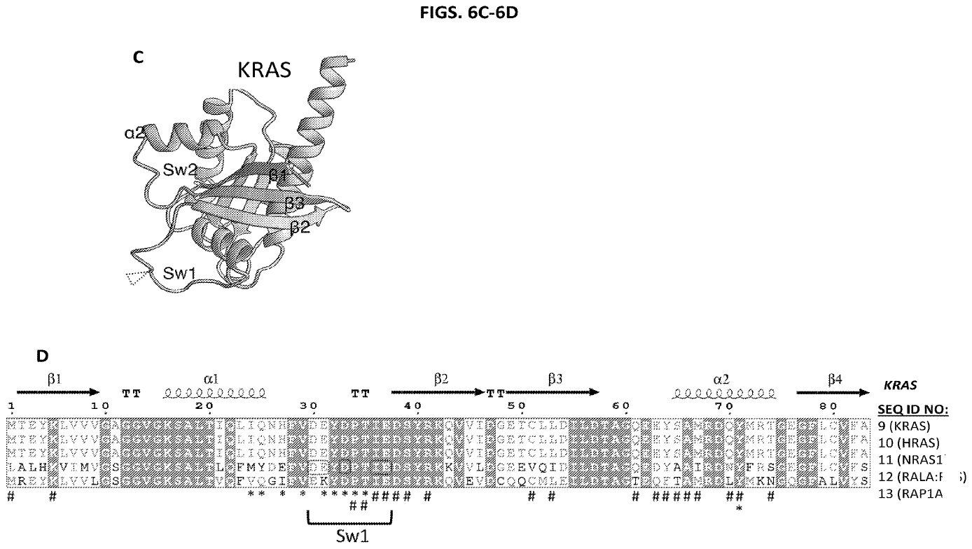

Intratumoral injection does not result in systemic spread of particles. To test if intratumoral injection would result in systemic spread of particles we tested if treatment of one tumor would result in reduction of a tumor on the opposing flank on the same mouse. We established two PANC-1 tumors per mouse and treated only one tumor. One tumor per mouse was treated with 5.5 μg rrsp mRNA in PPDP2. There was a significant reduction in tumor size for tumors in the PPDP2-rrsp treatment group but this treatement did not result in a reduction of the tumor in the opposing flank (FIGS. 5A-5B).

### Systemic Delivery of RRSP Exhibits Low Toxicity in DTR Knock-In Mice.

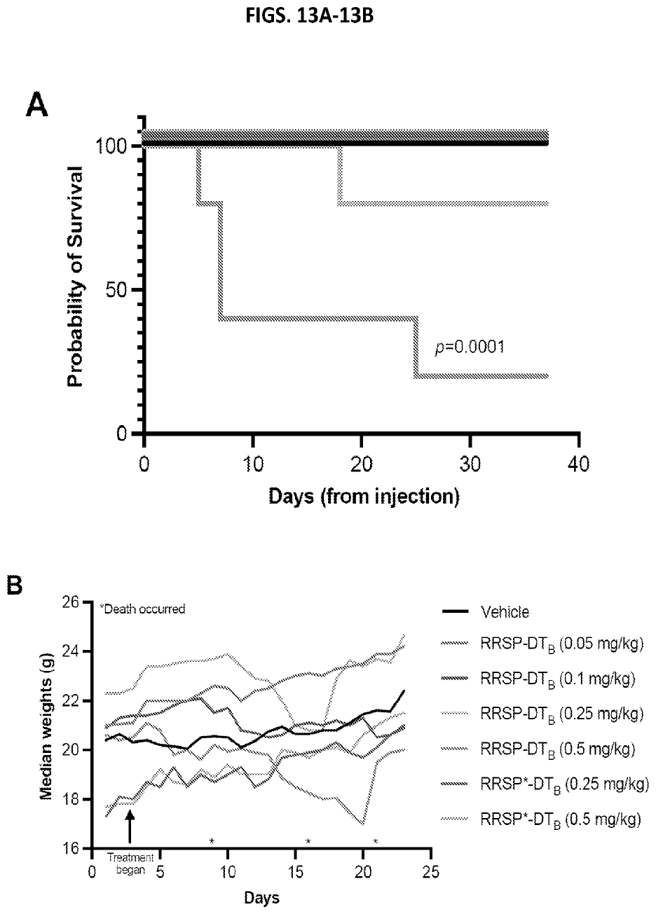

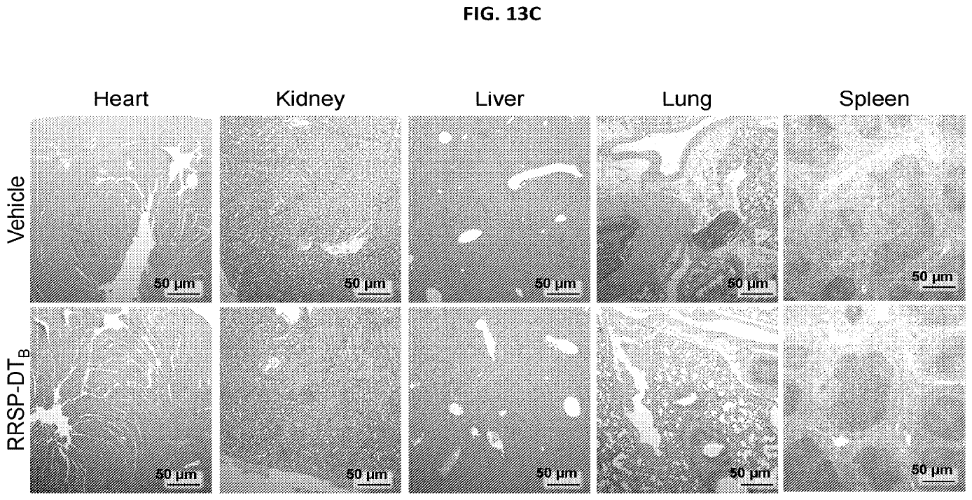

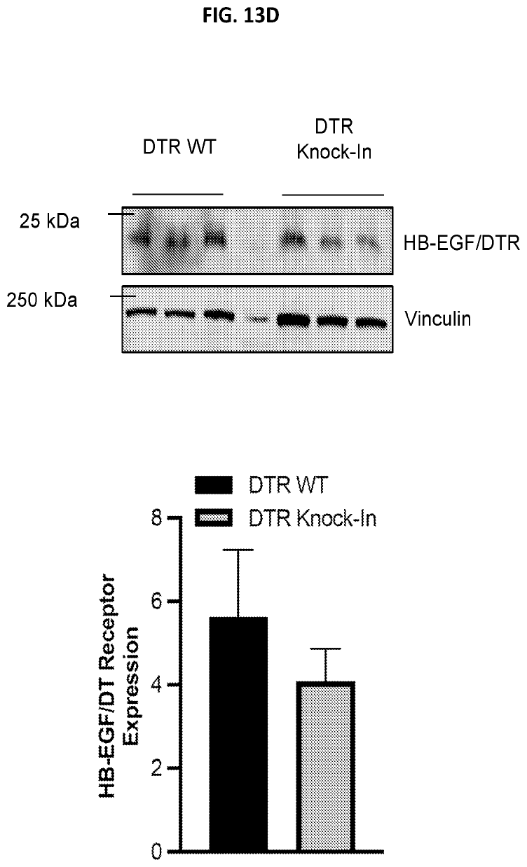

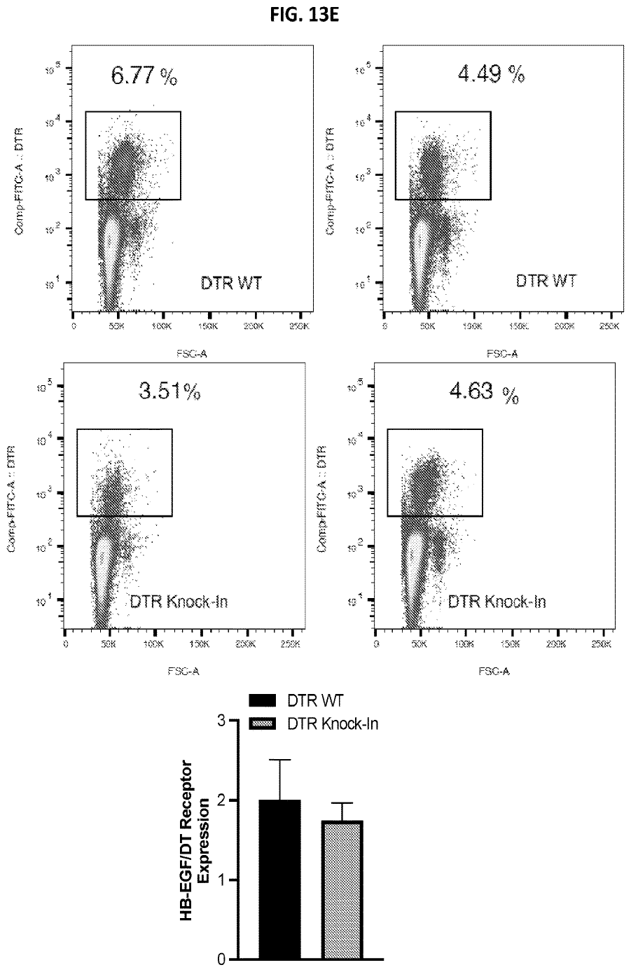

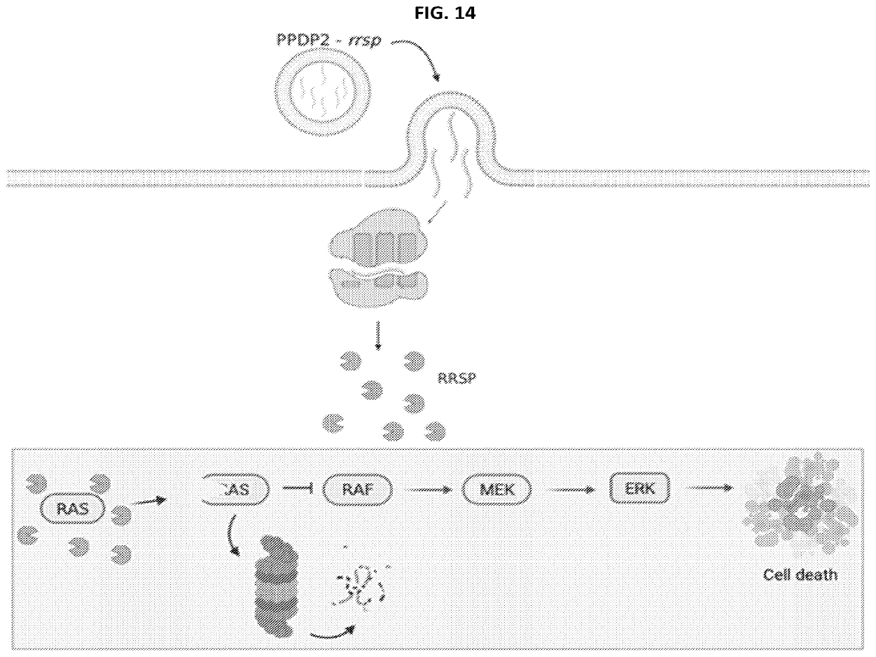

As our data only tested delivery of PPDP2 cargo upon intratumoral injection, we found it did not result in systemic spread to treat a tumor on the opposite flank. Regardless, the mice showed no evidence of toxicity (FIG. 5C), which was surprising as we assumed there would be some spread from the tumor injection site. Thus, we sought to explore if generalized toxicity of systemic RAS inhibition with RRSP would occur if RRSP was systemically delivered. We previously showed that systemic delivery of RRSP-DT_{B} (a protein fusion of RRSP fused with diphtheria toxin B subunit) can reduce tumors when specifically targeted to human cells, as mice lack a high affinity receptor for DT_{B} (20, 21). Herein, we explored the level of toxicity if the protein was accessible to all cells in the mouse. For this experiment, we created diphtheria toxin receptor (DTR) knock-in mice, expressing the high affinity human diphtheria toxin receptor HB-EGF in all cells, with the assistance of Jackson Laboratory. The DTR knock-in mice were treated with various doses up to 0.5 mg/kg of RRSP-DT_{B} (27) or catalytically inactive RRSP*-DT_{B} three times per week for 4 weeks. Mouse weights and general health were monitored, and deaths recorded (FIG. 12A). In the 0.5 mg/kg group, 3 mice died, and one mouse died from the 0.25 mg/kg group. No other deaths occurred, and mouse weights remained stable or increased throughout the experiment (FIGS. 13A-13B). Overall, we found the minimal tolerated dose (MTD) to be 0.25 mg/kg of RRSP delivered to all cells, the same MTD value previously found for normal mice where RRSP-DT_{B} is targeted to only human cells (20). Necropsy showed the treated mice without any significant changes to tissue architecture within the major organs or adverse effects on mouse weight, or general health, with only one death occurring over the course of the experiment (FIG. 13B). In addition, upon termination of the experiment, we validated the presence of DTR in the peripheral blood mononuclear cells (PBMC) compared to DTR WT mice (FIG. 13C). Overall, this shows systemic inhibition of all RAS is not toxic to mice, likely as RAS is only functional in actively growing cells.

### Structural Modeling of RRSP/RAS Indicate Low Risk of Resistance

A major concern currently for RAS-directed therapeutics is the development of resistance by gain of additional mutations in RAS (11) or increased expression of other rare RAS proteins such as MRAS (28). A major asset for RRSP is that it is a pan-RAS inhibitor that targets all isoforms of RAS so resistance is less likely to emerge by upregulation of a rare form of RAS unless the isoform gained a mutation that would resist binding of RRSP.

To consider the likelihood of RRSP resistant RAS mutations to arise, we used ColabFold integrated into ChimeraX to generate an AlphaFold2 (AF2) model of the dimeric structure of RRSP bound to KRAS. RRSP is a multidomain protein with a membrane targeting N-terminal domain C1 and a large C-terminal C2 domain formed as two lobes (termed C2A and C2B) joined by a long flexible helix. An initial analysis revealed that the RRSP membrane targeting C1 domain does not form contacts with the C2 domain (Table 1) consistent with removal of C1 not impacting cytotoxicity of RRSP (16). The AF2 model predicts that the C2 domain does bind with RAS with close contact (defined as <4 angstrom) at 26 distinct residues of the RAS G-domain. Residues 21-35 comprising the Switch 1 and neighboring residues are inserted into the active site of the C2B lobe with catalytic residues directed toward the scissile bond. However, unexpectedly, the majority of contacts between RAS and RRSP are not between the C2B catalytic lobe and the Switch 1 of KRAS. The primary site of recognition of KRAS is formed by the RAS beta-sheet (b1-b2-b3) as well as with Helix 2. This AF2 model is supported by prior data that overexpression of C2A alone in cells is cytotoxic, whereas overexpression of C2B alone is not cytotoxic in the absence of C2A (16). Hence the high specificity of RRSP for RAS is dictated outside of the Switch 1, even though there is sequence specificity for cleavage of the RAS Switch 1 (18). Further, the residues of the beta-sheet that contact RRSP are conserved across all RAS molecules, as well as RAP1. This very large area of contact sites suggests that a single mutation in RAS would not impede susceptibility of the molecule. Thus, the modeling supports that gain of additional mutations in RAS are unlikely to lead to resistance as any single point of contact is not likely essential and multiple mutations would be required for resistance. Indeed, one direct contact residue is Q61 in Switch 2 although a Q61R mutation remains susceptible to RRSP (16). Also, any single mutation in the Switch 1 did not impact susceptibility to RRSP (18). Thus, protein modeling supports our finding that RRSP is not likely to result in resistance from emergence of a KRAS mutation and as all RAS isoforms are cleaved in the cell.

TABLE 1
KRAS residues that make close contact with RRSP based on AF2 model

| KRAS | | RRSP | | | Distance | |
|---|---|---|---|---|---|---|
| residue | | residue | | Domain | (Å) | Interaction |
| Met | 1 | Pro | 225 | C2A | 3.99 | hydrophobic |
| Met | 1 | Ile | 226 | C2A | 3.41 | hydrophobic |
| Lys | 5 | Glu | 218 | C2A | 2.66 | salt bridge hydrogen bond |
| Ile | 24 | Asn | 401 | C2B | 3.60 | induction + dispersion |
| Ile | 24 | Gln | 403 | C2B | 3.89 | induction + dispersion |
| Gln | 25 | Arg | 399 | C2B | 3.78 | electrostatic |
| Gln | 25 | Asn | 401 | C2B | 3.18 | electrostatic |
| Gln | 25 | Gly | 404 | C2B | 3.28 | induction + dispersion |
| His | 27 | Arg | 399 | C2B | 3.07 | ionic repulsion CAUTION |
| His | 27 | Glu | 406 | C2B | 3.91 | salt bridge hydrogen bond anion-π stacking |
| His | 27 | Leu | 409 | C2B | 3.08 | induction + dispersion |
| Val | 29 | Arg | 399 | C2B | 3.07 | induction + dispersion |
| Glu | 31 | Leu | 409 | C2B | 3.76 | induction + dispersion |
| Glu | 31 | Ala | 440 | C2B | 3.03 | induction + dispersion |
| Glu | 31 | His | 445 | C2B | 3.37 | salt bridge hydrogen bond anion-π stacking |
| Tyr | 32 | His | 313 | C2B | 2.99 | cation-π stacking π-π stacking hydrogen bond |
| | | | | | | dipole-π stacking |
| Tyr | 32 | His | 342 | C2B | 3.72 | cation-π stacking π-π stacking hydrogen bond |
| | | | | | | dipole-π stacking |
| Tyr | 32 | Lys | 372 | C2B | 3.46 | cation-π stacking hydrogen bond |
| Asp | 33 | Arg | 399 | C2B | 3.35 | salt bridge hydrogen bond |
| Pro | 34 | Gln | 201 | C2A | 3.84 | induction + dispersion |
| Pro | 34 | Arg | 344 | C2B | 3.43 | induction + dispersion |
| Pro | 34 | Lys | 372 | C2B | 2.81 | induction + dispersion |
| Pro | 34 | Asp | 393 | C2B | 3.21 | induction + dispersion |
| Pro | 34 | Arg | 412 | C2B | 3.31 | induction + dispersion |
| Thr | 35 | Gln | 198 | C2A | 3.77 | electrostatic |
| Thr | 35 | Gln | 201 | C2A | 3.59 | electrostatic |
| Thr | 35 | Arg | 344 | C2B | 2.89 | electrostatic |
| Thr | 35 | Ala | 398 | C2B | 3.81 | induction + dispersion |
| Ile | 36 | Gln | 201 | C2A | 2.64 | induction + dispersion |
| Glu | 37 | Tyr | 197 | C2A | 2.65 | anion-π stacking π-π stacking electrostatic |
| Glu | 37 | Gln | 201 | C2A | 3.48 | electrostatic |
| Glu | 37 | Ser | 230 | C2A | 3.18 | electrostatic |
| Glu | 37 | Ala | 231 | C2A | 3.71 | induction + dispersion |
| Glu | 37 | Glu | 232 | C2A | 3.88 | ionic repulsion CAUTION |
| Asp | 38 | Lys | 217 | C2A | 2.57 | salt bridge hydrogen bond |
| Ser | 39 | Lys | 217 | C2A | 3.57 | electrostatic |
| Arg | 41 | Lys | 221 | C2A | 3.76 | ionic repulsion CAUTION |
| Arg | 41 | Asp | 224 | C2A | 2.91 | salt bridge hydrogen bond |
| Arg | 41 | Ser | 227 | C2A | 3.00 | electrostatic |
| Arg | 41 | Gly | 228 | C2A | 3.26 | induction + dispersion |
| Arg | 41 | Asp | 229 | C2A | 2.74 | salt bridge hydrogen bond |
| Arg | 41 | Ile | 234 | C2A | 2.94 | induction + dispersion |
| Leu | 52 | Pro | 225 | C2A | 3.44 | hydrophobic |
| Leu | 52 | Ser | 227 | C2A | 3.67 | induction + dispersion |
| Leu | 52 | Gly | 228 | C2A | 3.93 | hydrophobic |
| Asp | 54 | Lys | 221 | C2A | 2.74 | salt bridge hydrogen bond |
| Gln | 61 | Gln | 210 | C2A | 2.74 | electrostatic |
| Glu | 63 | Lys | 370 | C2B | 2.67 | salt bridge hydrogen bond |
| Tyr | 64 | Thr | 204 | C2A | 3.73 | electrostatic |
| Tyr | 64 | Gly | 205 | C2A | 2.78 | hydrophobic |
| Tyr | 64 | Glu | 207 | C2A | 3.06 | anion-π stacking π-π stacking electrostatic |
| Tyr | 64 | Gln | 210 | C2A | 2.90 | electrostatic |
| Tyr | 64 | Lys | 370 | C2B | 2.83 | cation-π stacking hydrogen bond |
| Ser | 65 | Glu | 207 | C2A | 2.95 | electrostatic |
| Ala | 66 | Trp | 169 | C2A | 4.00 | hydrophobic |
| Ala | 66 | Glu | 207 | C2A | 2.75 | induction + dispersion |
| Ala | 66 | Met | 211 | C2A | 3.83 | hydrophobic |
| Met | 67 | Glu | 207 | C2A | 3.59 | electrostatic |
| Met | 67 | Gln | 210 | C2A | 3.31 | electrostatic |
| Met | 67 | Met | 211 | C2A | 3.41 | hydrophobic electrostatic |
| Met | 67 | Tyr | 214 | C2A | 3.27 | electrostatic |
| Gln | 70 | Met | 211 | C2A | 3.97 | electrostatic |
| Gln | 70 | Tyr | 214 | C2A | 3.48 | electrostatic |
| Tyr | 71 | Tyr | 214 | C2A | 3.51 | hydrogen bond π-π stacking dipole-π stacking |
| | | | | | | hydrophobic |
| Thr | 74 | Tyr | 214 | C2A | 3.04 | electrostatic |

### DISCUSSION

Uncontrolled mutant KRAS signaling drives the onset and progression of 95% of pancreatic cancers (29). Although many mutant KRAS inhibitors exist, none are currently approved for use in PDAC (11). Recently, Sotorasib, a KRAS G12C inhibitor, has shown clinical activity in heavily pretreated patients with KRAS G12C-mutated metastatic pancreatic cancer (30). However, KRAS G12C mutations are only present in 1-2% of PDAC cases. Therefore, a need exists for a pan-RAS inhibitor that is effective against all RAS isoforms and variants and that avoids drug resistance.

RRSP is unique amongst the current RAS degraders and is more advanced in understanding of its mechanism and methods of delivery. First, most RAS degraders are selective against only KRAS or even one specific mutation, such as ASP3082 which targets only KRAS G12D (31). RRSP cleaves HRAS, NRAS, and KRAS as well as KRAS G12V, G12D, G12C, G13D, and Q61R mutants and both GDP- and GTP-bound RAS (16-19). It also demonstrates complete degradation of RAS in the picomolar range, outperforming other degraders under investigation. In addition, the majority of RAS degraders have been shown to be effective using transfection strategies or doxycycline-inducible expression. RRSP is the only degrader that reduced tumors by both exogenous protein addition and mRNA delivery and that has shown in vivo efficacy across several different models, including breast, colon, and pancreatic mouse models (11).

Our data herein demonstrate the efficacy of using synthetic nanoparticles for mRNA delivery to achieve intracellular expression of RRSP, and the potential use of this combination approach for cancer therapy. This approach is supported further by data indicating lipid nanoparticles were successful in delivering rrsp mRNA into various cancer cell lines and intracellular expression of functional RRSP. In addition, this combination suppressed tumor growth (25). Both nanoparticle delivery methods allowed rrsp mRNA to escape from the endosome and expression of RRSP protein was detected in tumors. In addition, lipid nanoparticle delivery of rrsp mRNA was based on higher reactive oxygen species (ROS) in the tumor microenvironment (25), while our system does not need this specific environment to deliver rrsp mRNA. Furthermore, synthetic polymers can be modified to respond to a variety of different chemical, or environmental triggers to enhance delivery. PEG-PPS has been specifically shown to respond to ROS, which has advantages for targeting cancer and innate immune cells. PEG-PPS nanoparticles can also be engineered to respond to specific wavelengths of light and ultrasound. These capabilities are much more difficult to achieve with LNPs.

Both methods of rrsp mRNA delivery with nanoparticles are efficacious and warrant further investigation to treat RAS-driven cancers. Surprisingly, our data indicate that the catalytically inactive mutant of RRSP (RRSP*) has enough physical contacts with RAS to inhibit its activity and cause tumor regression. The data are consistent with our prior work that C2A alone is sufficient to cause cell cytotoxicity (16). Further a homolog of RRSP from Photorhabdus luminescence also induced cell arrest, even when the protein was catalytically inactive (32). Our previous work has further shown reduction in pERK levels with 10 nM of RRSP* through protein delivery (20). With intratumoral delivery, this study demonstrated only 10 μM rrsp*mRNA produced enough active RRSP* to sufficiently reduce pERK levels and tumor growth.

Although we initially tested targeted delivery of RRSP by intratumoral injection, we went on to investigate the toxicity of RRSP delivery to all cells. Even with untargeted delivery, no toxicity was shown with RRSP protein at 1 mg/kg (10 nM) doses and limited toxicity up to 2.5 mg/kg (25 nM). We saw no detrimental effects on the animal health through either experiment. The mouse weights remained stable and even increased throughout the study and major organs showed no morphological changes. While preliminary, these studies indicate that systemic loss of RAS is unexpectedly low toxicity (FIG. 13). We hypothesize that we observed limited toxicity because RAS is most important in actively growing cells and thus terminally differentiated and non-growing cells may not be severely impacted by the loss of RAS. We have previously shown that RRSP degradation of RAS leads to G1 cell cycle arrest that can progress to apoptosis, senescence, and loss of cell proliferation depending on the cell line, with those exhibiting RAS genomic abnormalities being the most sensitive (21). These results are supported by recent reports from Revolution Medicine that their anti-pan-RAS small molecule therapeutic is effective, safe, and tolerated in humans (33, 34). This is in sharp contrast to MEK inhibitors which have shown off-target effects and toxicity (35, 36). This difference in toxicity is perhaps due to MEK crosstalk with AKT, mammalian target of rapamycin (mTOR) and Rac1 (37, 38). We were surprised to find this lack of toxicity of RRSP further supporting its potential in cancer therapy.

A major limitation to cancer therapeutics is the development of resistance. Current KRAS therapeutic strategies exhibit resistance through a variety of genomic and signaling mechanisms (39). Tumors can develop KRAS-independent signaling, mutations in other oncogenic drivers, activation of other RAS isoforms, and other methods which contribute to resistance (4, 40-44). In a prior study with RRSP-DT_{B}, we did not observe tumor resistance to a second treatment. In this study, we likewise found no emergence of resistance and a tumor that was previously treated regressed while a control tumor on the alternative flank continued to expand. The lack of emerging resistance is strongly supported by AF2 modeling of interface between RRSP and RAS. RRSP specifically contacts RAS both through the catalytic C2B domain that binds Switch 1 region and also with C2A contact of the RAS beta-sheet. It is known that single point mutations in RAS do not impact processing by RRSP indicating that multiple mutations in oncogenic RAS would be required for resistance to develop.

Overall, RRSP continues to be demonstrated as a potent Ras Degrader functional both in vitro and in vivo. It has been shown to reduce tumors by three delivery strategies and to be useful against all types of cancer including breast, colon, and pancreatic. Further, our findings here that the mRNA delivery by PPDP2 is so highly effective that protein expression in tumor cells was sufficiently high for even catalytically inactive protein to inhibit tumor growth. Finally, the systemic delivery of RRSP was no more toxic to mice that have the HB-EGF receptor. These data support both protein and mRNA based delivery strategies for delivery of RRSP could progress into the next stage of preclinical and clinical development, potentially without need to develop complex tumor specific targeting strategies. Overall, RRSP is well poised for development as cancer gene therapeutic, particularly when paired with synthetic nanoparticles for mRNA delivery.

### Materials and Methods

### Chemicals, Protein Purification, and Cell Lines

All chemicals were from Sigma-Aldrich unless otherwise specified. An mRNA for expression of GFP, a second CleanCap eGFP-mRNA and an mRNA for expression of the RAS/Rap1 protease (also known as Vibrio vulnificus MARTX toxin DUF5) (rrsp-mRNA) and a catalytically inactive negative control mRNA (rrsp*mRNA) were synthesized by TriLink BioTechnologies. mRNA sequences were capped with 5′ AG head (CleanCap®), hemagglutinin (HA) tagged (as indicated) and polyadenylated tail and 100% pseudouridine. Sequences for all mRNA are found as Table 2.

TABLE 2

| mRNA sequences (5′-3′) | | SEQ ID NO: |
|---|---|---|
| egfp mRNA | AUGGUGAGC0AAGGGCGAGGAGCUGUUCACCGGGGUG | 1 |
| | GUGCCCAUCCUGGUCGAGCUGGACGGCGACGUAAACG | |
| | GCCACAAGUUCAGCGUGUCCGGCGAGGGCGAGGGCGA | |
| | UGCCACCUACGGCAAGCUGACCCUGAAGUUCAUCUGC | |
| | ACCACCGGCAAGCUGCCCGUGCCCUGGCCCACCCUCG | |
| | UGACCACCCUGACCUACGGCGUGCAGUGCUUCAGCCG | |
| | CUACCCCGACCACAUGAAGCAGCACGACUUCUUCAAG | |
| | UCCGCCAUGCCCGAAGGCUACGUCCAGGAGCGCACCA | |
| | UCUUCUUCAAGGACGACGGCAACUACAAGACCCGCGC | |
| | CGAGGUGAAGUUCGAGGGCGACACCCUGGUGAACCGC | |
| | AUCGAGCUGAAGGGCAUCGACUUCAAGGAGGACGGC | |
| | AACAUCCUGGGGCACAAGCUGGAGUACAACUACAACA | |
| | GCCACAACGUCUAUAUCAUGGCCGACAAGCAGAAGAA | |
| | CGGCAUCAAGGUGAACUUCAAGAUCCGCCACAACAUC | |
| | GAGGACGGCAGCGUGCAGCUCGCCGACCACUACCAGC | |
| | AGAACACCCCCAUCGGCGACGGCCCCGUGCUGCUGCC | |
| | CGACAACCACUACCUGAGCACCCAGUCCGCCCUGAGC | |
| | AAAGACCCCAACGAGAAGCGCGAUCACAUGGUCCUGC | |
| | UGGAGUUCGUGACCGCCGCCGGGAUCACUCUCGGCAU | |
| | GGACGAGCUGUACAAGUAA | |
| rrsp mRNA - | AUGUACCCAUACGAUGUUCCAGAUUACGCUGGUGAU | 2 |
| HA Tag | AAAACCAAGGUCGUGGUCGAUUUAGCGCAAAUCUUU | |
| | ACGGUGCAAGAGCUGAAAGAAAGAGCAAAAGUUUUU | |
| | GCUAAACCGAUUGGCGCAUCCUACCAAGGUAUUCUCG | |
| | AUCAACUCGACCUUGUGCAUCAGGCUAAAGGCCGCGA | |
| | UCAAAUCGCAGCGAGCUUUGAGCUUAAUAAGAAGAU | |
| | UAAUGACUACAUCGCUGAACAUCCAACUUCGGGGCGU | |
| | AAUCAAGCGCUAACGCAGUUGAAAGAGCAGGUCACC | |
| | AGUGCGUUGUUUAUCGGUAAGAUGCAAGUUGCCCAA | |
| | GCGGGUAUUGAUGCAAUCGCACAAACAAGACCGGAG | |
| | CUUGCCGCUCGUAUCUUUAUGGUCGCGAUUGAAGAA | |
| | GCCAACGGUAAACACGUAGGUUUGACGGACAUGAUG | |
| | GUUCGUUGGGCCAAUGAAGACCCAUACUUGGCACCGA | |
| | AGCAUGGUUACAAAGGCGAAACGCCAAGUGACCUUG | |
| | GUUUUGAUGCGAAGUACCACGUAGAUCUAGGUGAGC | |
| | AUUACGCUGAUUUCAAACAGUGGUUAGAAACGUCCC | |
| | AGUCGAACGGGUUGUUGAGUAAAGCGACGUUGGAUG | |
| | AAUCCACUAAAACGGUUCAUCUUGGCUAUAGCUAUC | |
| | AAGAACUUCAGGAUUUGACGGGUGCUGAAUCGGUGC | |
| | AAAUGGCGUUCUACUUCCUGAAAGAAGCGGCGAAGA | |
| | AAGCGGAUCCGAUUUCUGGUGAUUCAGCUGAAAUGA | |
| | UACUGCUGAAGAAAUUUGCAGAUCAAAGCUACUUAU | |
| | CUCAACUUGAUUCCGACCGAAUGGAUCAAAUUGAAG | |
| | GUAUCUACCGCAGUAGCCAUGAGACGGAUAUUGACG | |
| | CUUGGGAUCGUCGUUACUCUGGUACAGGCUAUGAUG | |
| | AGCUGACGAAUAAGCUUGCUAGUGCAACGGGCGUUG | |
| | ACGAGCAGCUUGCGGUUCUUCUGGAUGAUCGUAAAG | |
| | GCCUCUUGAUUGGUGAAGUGCAUGGCAGCGACGUCA | |
| | ACGGCCUACGCUUUGUUAAUGAACAGAUGGAUGCAC | |
| | UGAAAAAACAGGGAGUCACAGUCAUUGGCCUUGAGC | |
| | AUUUACGCUCAGACCUUGCGCAACCGCUGAUUGAUCG | |
| | CUACCUAGCUACGGGUGUGAUGUCGAGUGAACUAAG | |
| | CGCAAUGCUGAAAACAAAGCAUCUCGAUGUCACUCUU | |
| | UUUGAAAACGCACGUGCUAACGGUAUGCGCAUCGUC | |
| | GCGCUGGAUGCAAACAGCUCUGCGCGUCCAAAUGUUC | |
| | AGGGAACAGAACAUGGUCUGAUGUACCGUGCUGGUG | |
| | CUGCGAACAACAUUGCGGUGGAAGUAUUACAAAAUC | |
| | UGCCUGAUGGCGAAAAGUUCGUUGCUAUCUACGGUA | |
| | AAGCGCAUUUGCAGUCUCACAAAGGGAUUGAAGGGU | |
| | UCGUUCCUGGUAUCACGCACCGUCUCGAUCUUCCUGC | |
| | GCUUAAAGUCAGUGACUCGAACCAGUUCACAGUUGA | |
| | ACAAGACGAUGUAAGUCUACGUGUUGUCUACGAUGA | |
| | UGUUGCUAACAAACCGAAGAUCACGUUCAAGGGCAG | |
| | UUUGUAG | |
| rrsp* mRNA - | AUGUACCCAUACGUUCCAGAUUACGCUAUGGGUGAU | 3 |
| HA Tag | AAAACCAAGGUCGUGGUCGAUUUAGCGCAAAUCUUU | |
| | ACGGUGCAAGAGCUGAAAGAAAGAGCAAAAGUUUUU | |
| | GCUAAACCGAUUGGCGCAUCCUACCAAGGUAUUCUCG | |
| | AUCAACUCGACCUUGUGCAUCAGGCUAAAGGCCGCGA | |
| | UCAAAUCGCAGCGAGCUUUGAGCUUAAUAAGAAGAU | |
| | UAAUGACUACAUCGCUGAACAUCCAACUUCGGGGCGU | |
| | AAUCAAGCGCUAACGCAGUUGAAAGAGCAGGUCACC | |
| | AGUGCGUUGUUUAUCGGUAAGAUGCAAGUUGCCCAA | |
| | GCGGGUAUUGAUGCAAUCGCACAAACAAGACCGGAG | |
| | CUUGCCGCUCGUAUCUUUAUGGUCGCGAUUGAAGAA | |
| | GCCAACGGUAAACACGUAGGUUUGACGGACAUGAUG | |
| | GUUCGUUGGGCCAAUGAAGACCCAUACUUGGCACCGA | |
| | AGCAUGGUUACAAAGGCGAAACGCCAAGUGACCUUG | |
| | GUUUUGAUGCGAAGUACCACGUAGAUCUAGGUGAGC | |
| | AUUACGCUGAUUUCAAACAGUGGUUAGAAACGUCCC | |
| | AGUCGAACGGGUUGUUGAGUAAAGCGACGUUGGAUG | |
| | AAUCCACUAAAACGGUUCAUCUUGGCUAUAGCUAUC | |
| | AAGAACUUCAGGAUUUGACGGGUGCUGAAUCGGUGC | |
| | AAAUGGCGUUCUACUUCCUGAAAGAAGCGGCGAAGA | |
| | AAGCGGAUCCGAUUUCUGGUGAUUCAGCUGAAAUGA | |
| | UACUGCUGAAGAAAUUUGCAGAUCAAAGCUACUUAU | |
| | CUCAACUUGAUUCCGACCGAAUGGAUCAAAUUGAAG | |
| | GUAUCUACCGCAGUAGCCAUGAGACGGAUAUUGACG | |
| | CUUGGGAUCGUCGUUACUCUGGUACAGGCUAUGAUG | |
| | AGCUGACGAAUAAGCUUGCUAGUGCAACGGGCGUUG | |
| | ACGAGCAGCUUGCGGUUCUUCUGGAUGAUCGUAAAG | |
| | GCCUCUUGAUUGGUGAAGUGCAUGGCAGCGACGUCA | |
| | ACGGCCUACGCUUUGUUAAUGAACAGAUGGAUGCAC | |
| | UGAAAAAACAGGGAGUCACAGUCAUUGGCCUUGAGC | |
| | AUUUACGCUCAGACCUUGCGCAACCGCUGAUUGAUCG | |
| | CUACCUAGCUACGGGUGUGAUGUCGAGUGAACUAAG | |
| | CGCAAUGCUGAAAACAAAGCAUCUCGAUGUCACUCUU | |
| | UUUGAAAACGCACGUGCUAACGGUAUGCGCAUCGUC | |
| | GCGCUGGAUGCAAACAGCUCUGCGCGUCCAAAUGUUC | |
| | AGGGAACAGAACAUGGUCUGAUGUACCGUGCUGGUG | |
| | CUGCGAACAACAUUGCGGUGGAAGUAUUACAAAAUC | |
| | UGCCUGAUGGCGAAAAGUUCGUUGCUAUCUACGGUA | |
| | AAGCGCAUUUGCAGUCUCACAAAGGGAUUGAAGGGU | |
| | UCGUUCCUGGUAUCACGGCGCGUCUCGAUCUUCCUGC | |
| | GCUUAAAGUCAGUGACUCGAACCAGUUCACAGUUGA | |
| | ACAAGACGAUGUAAGUCUACGUGUUGUCUACGAUGA | |
| | UGUUGCUAACAAACCGAAGAUCACGUUCAAGGGCAG | |
| | UUUGUAG | |
| rrsp mRNA - | AUGGGUGAUAAAACCAAGGUCGUGGUCGAUUUAGCG | 4 |
| no tag | CAAAUCUUUACGGUGCAAGAGCUGAAAGAAAGAGCA | |
| | AAAGUUUUUGCUAAACCGAUUGGCGCAUCCUACCAA | |
| | GGUAUUCUCGAUCAACUCGACCUUGUGCAUCAGGCUA | |
| | AAGGCCGCGAUCAAAUCGCAGCGAGCUUUGAGCUUA | |
| | AUAAGAAGAUUAAUGACUACAUCGCUGAACAUCCAA | |
| | CUUCGGGGCGUAAUCAAGCGCUAACGCAGUUGAAAG | |
| | AGCAGGUCACCAGUGCGUUGUUUAUCGGUAAGAUGC | |
| | AAGUUGCCCAAGCGGGUAUUGAUGCAAUCGCACAAA | |
| | CAAGACCGGAGCUUGCCGCUCGUAUCUUUAUGGUCGC | |
| | GAUUGAAGAAGCCAACGGUAAACACGUAGGUUUGAC | |
| | GGACAUGAUGGUUCGUUGGGCCAAUGAAGACCCAUA | |
| | CUUGGCACCGAAGCAUGGUUACAAAGGCGAAACGCCA | |
| | AGUGACCUUGGUUUUGAUGCGAAGUACCACGUAGAU | |
| | CUAGGUGAGCAUUACGCUGAUUUCAAACAGUGGUUA | |
| | GAAACGUCCCAGUCGAACGGGUUGUUGAGUAAAGCG | |
| | ACGUUGGAUGAAUCCACUAAAACGGUUCAUCUUGGC | |
| | UAUAGCUAUCAAGAACUUCAGGAUUUGACGGGUGCU | |
| | GAAUCGGUGCAAAUGGCGUUCUACUUCCUGAAAGAA | |
| | GCGGCGAAGAAAGCGGAUCCGAUUUCUGGUGAUUCA | |
| | GCUGAAAUGAUACUGCUGAAGAAAUUUGCAGAUCAA | |
| | AGCUACUUAUCUCAACUUGAUUCCGACCGAAUGGAUC | |
| | AAAUUGAAGGUAUCUACCGCAGUAGCCAUGAGACGG | |
| | AUAUUGACGCUUGGGAUCGUCGUUACUCUGGUACAG | |
| | GCUAUGAUGAGCUGACGAAUAAGCUUGCUAGUGCAA | |
| | CGGGCGUUGACGAGCAGCUUGCGGUUCUUCUGGAUG | |
| | AUCGUAAAGGCCUCUUGAUUGGUGAAGUGCAUGGCA | |
| | GCGACGUCAACGGCCUACGCUUUGUUAAUGAACAGA | |
| | UGGAUGCACUGAAAAAACAGGGAGUCACAGUCAUUG | |
| | GCCUUGAGCAUUUACGCUCAGACCUUGCGCAACCGCU | |
| | GAUUGAUCGCUACCUAGCUACGGGUGUGAUGUCGAG | |
| | UGAACUAAGCGCAAUGCUGAAAACAAAGCAUCUCGA | |
| | UGUCACUCUUUUUGAAAACGCACGUGCUAACGGUAU | |
| | GCGCAUCGUCGCGCUGGAUGCAAACAGCUCUGCGCGU | |
| | CCAAAUGUUCAGGGAACAGAACAUGGUCUGAUGUAC | |
| | CGUGCUGGUGCUGCGAACAACAUUGCGGUGGAAGUA | |
| | UUACAAAAUCUGCCUGAUGGCGAAAAGUUCGUUGCU | |
| | AUCUACGGUAAAGCGCAUUUGCAGUCUCACAAAGGG | |
| | AUUGAAGGGUUCGUUCCUGGUAUCACGCACCGUCUCG | |
| | AUCUUCCUGCGCUUAAAGUCAGUGACUCGAACCAGUU | |
| | CACAGUUGAACAAGACGAUGUAAGUCUACGUGUUGU | |
| | CUACGAUGAUGUUGCUAACAAACCGAAGAUCACGUU | |
| | CAAGGGCAGUUUGUAG | |
| mCherry | AUGGUGAGCAAGGGCGAGGAGGACAACAUGGCCAUC | 5 |
| mRNA | AUCAAGGAGUUCAUGCGGUUCAAGGUGCACAUGGAG | |
| | GGCAGCGUGAACGGCCACGAGUUCGAGAUCGAGGGC | |
| | GAGGGCGAGGGCCGGCCCUACGAGGGCACCCAGACCG | |
| | CCAAGCUGAAGGUGACCAAGGGCGGCCCCCUGCCCUU | |
| | CGCCUGGGACAUCCUGAGCCCCCAGUUCAUGUACGGC | |
| | AGCAAGGCCUACGUGAAGCACCCCGCCGACAUCCCCG | |
| | ACUACCUGAAGCUGAGCUUCCCCGAGGGCUUCAAGUG | |
| | GGAGCGGGUGAUGAACUUCGAGGACGGCGGCGUGGU | |
| | GACCGUGACCCAGGACAGCAGCCUGCAGGACGGCGAG | |
| | UUCAUCUACAAGGUGAAGCUGCGGGGCACCAACUUCC | |
| | CCAGCGACGGCCCCGUGAUGCAGAAGAAGACCAUGGG | |
| | CUGGGAGGCCAGCAGCGAGCGGAUGUACCCCGAGGAC | |
| | GGCGCCCUGAAGGGCGAGAUCAAGCAGCGGCUGAAGC | |
| | UGAAGGACGGCGGCCACUACGACGCCGAGGUGAAGAC | |
| | CACCUACAAGGCCAAGAAGCCCGUGCAGCUGCCCGGC | |
| | GCCUACAACGUGAACAUCAAGCUGGACAUCACCAGCC | |
| | ACAACGAGGACUACACCAUCGUGGAGCAGUACGAGCG | |
| | GGCCGAGGGCCGGCACAGCACCGGCGGCAUGGACGAG | |
| | CUGUACAAGAGCGGCAACUGA | |
| Plasmid | GTACATGACCTTATGGGACTTTCCTACTTGGCAGTACA | 6 |
| DUFVvC2- | TCTACGTATTAGTCATCGCTATTACCATGGTGATGCGGT | |
| EGFP | TTTGGCAGTACATCAATGGGCGTGGATAGCGGTTTGAC | |
| | TCACGGGGATTTCCAAGTCTCCACCCCATTGACGTCAA | |
| | TGGGAGTTTGTTTTGGCACCAAAATCAACGGGACTTTC | |
| | CAAAATGTCGTAACAACTCCGCCCCATTGACGCAAATG | |
| | GGCGGTAGGCGTGTACGGTGGGAGGTCTATATAAGCA | |
| | GAGCTGGTTTAGTGAACCGTCAGATCCGCTAGCCGCCA | |
| | CCATGTTTATCGGTAAGATGCAAGTTGCCCAAGCGGGT | |
| | ATTGATGCAATCGCACAAACAAGACCGGAGCTTGCCGC | |
| | TCGTATCTTTATGGTCGCGATTGAAGAAGCCAACGGTA | |
| | AACACGTAGGTTTGACGGACATGATGGTTCGTTGGGCC | |
| | AATGAAGACCCATACTTGGCACCGAAGCATGGTTACAA | |
| | AGGCGAAACGCCAAGTGACCTTGGTTTTGATGCGAAGT | |
| | ACCACGTAGATCTAGGTGAGCATTACGCTGATTTCAAA | |
| | CAGTGGTTAGAAACGTCCCAGTCGAACGGGTTGTTGAG | |
| | TAAAGCGACGTTGGATGAATCCACTAAAACGGTTCATC | |
| | TTGGCTATAGCTATCAAGAACTTCAGGATTTGACGGGT | |
| | GCTGAATCGGTGCAAATGGCGTTCTACTTCCTGAAAGA | |
| | AGCGGCGAAGAAAGCGGATCCGATTTCTGGTGATTCAG | |
| | CTGAAATGATACTGCTGAAGAAATTTGCAGATCAAAGC | |
| | TACTTATCTCAACTTGATTCCGACCGAATGGATCAAAT | |
| | TGAAGGTATCTACCGCAGTAGCCATGAGACGGATATTG | |
| | ACGCTTGGGATCGTCGTTACTCTGGTACAGGCTATGAT | |
| | GAGCTGACGAATAAGCTTGCTAGTGCAACGGGCGTTGA | |
| | CGAGCAGCTTGCGGTTCTTCTGGATGATCGTAAAGGCC | |
| | TCTTGATTGGTGAAGTGCATGGCAGCGACGTCAACGGC | |
| | CTACGCTTTGTTAATGAACAGATGGATGCACTGAAAAA | |
| | ACAGGGAGTCACAGTCATTGGCCTTGAGCATTTACGCT | |
| | CAGACCTTGCGCAACCGCTGATTGATCGCTACCTAGCT | |
| | ACGGGTGTGATGTCGAGTGAACTAAGCGCAATGCTGA | |
| | AAACAAAGCATCTCGATGTCACTCTTTTTGAAAACGCA | |
| | CGTGCTAACGGTATGCGCATCGTCGCGCTGGATGCAAA | |
| | CAGCTCTGCGCGTCCAAATGTTCAGGGAACAGAACATG | |
| | GTCTGATGTACCGTGCTGGTGCTGCGAACAACATTGCG | |
| | GTGGAAGTATTACAAAATCTGCCTGATGGCGAAAAGTT | |
| | CGTTGCTATCTACGGTAAAGCGCATTTGCAGTCTCACA | |
| | AAGGGATTGAAGGGTTCGTTCCTGGTATCACGCACCGT | |
| | CTCGATCTTCCTGCGCTTAAAGTCAGTGACTCGAACCA | |
| | GTTCACAGTTGAACAAGACGATGTAAGTCTACGTGTTG | |
| | TCTACGATGATGTTGCTAACAAACCGAAGATCACGTTC | |
| | AAGGGCAGTTTGGTCGACGGTACCGCGGGCCCGGGAT | |
| | CCATCGCCACCATGGTGAGCAAGGGCGAGGAGCTGTTC | |
| | ACCGGGGTGGTGCCCATCCTGGTCGAGCTGGACGGCGA | |
| | CGTAAACGGCCACAAGTTCAGCGTGTCCGGCGAGGGC | |
| | GAGGGCGATGCCACCTACGGCAAGCTGACCCTGAAGTT | |
| | CATCTGCACCACCGGCAAGCTGCCCGTGCCCTGGCCCA | |
| | CCCTCGTGACCACCCTGACCTACGGCGTGCAGTGCTTC | |
| | AGCCGCTACCCCGACCACATGAAGCAGCACGACTTCTT | |
| | CAAGTCCGCCATGCCCGAAGGCTACGTCCAGGAGCGC | |
| | ACCATCTTCTTCAAGGACGACGGCAACTACAAGACCCG | |
| | CGCCGAGGTGAAGTTCGAGGGCGACACCCTGGTGAAC | |
| | CGCATCGAGCTGAAGGGCATCGACTTCAAGGAGGACG | |
| | GCAACATCCTGGGGCACAAGCTGGAGTACAACTACAA | |
| | CAGCCACAACGTCTATATCATGGCCGACAAGCAGAAG | |
| | AACGGCATCAAGGTGAACTTCAAGATCCGCCACAACAT | |
| | CGAGGACGGCAGCGTGCAGCTCGCCGACCACTACCAG | |
| | CAGAACACCCCCATCGGCGACGGCCCCGTGCTGCTGCC | |
| | CGACAACCACTACCTGAGCACCCAGTCCGCCCTGAGCA | |
| | AAGACCCCAACGAGAAGCGCGATCACATGGTCCTGCT | |
| | GGAGTTCGTGACCGCCGCCGGGATCACTCTCGGCATGG | |
| | ACGAGCTGTACAAGTAAAGCGGCCGCGACTCTAGATC | |
| | ATAATCAGCCATACCACATTTGTAGAGGTTTTACTTGCT | |
| | TTAAAAAACCTCCCACACCTCCCCCTGAACCTGAAACA | |
| | TAAAATGAATGCAATTGTTGTTGTTAACTTGTTTATTGC | |
| | AGCTTATAATGGTTACAAATAAAGCAATAGCATCACAA | |
| | ATTTCACAAATAAAGCATTTTTTTCACTGCATTCTAGTT | |
| | GTGGTTTGTCCAAACTCATCAATGTATCTTAAGGCGTA | |
| | AATTGTAAGCGTTAATATTTTGTTAAAATTCGCGTTAA | |
| | ATTTTTGTTAAATCAGCTCATTTTTTAACCAATAGGCCG | |
| | AAATCGGCAAAATCCCTTATAAATCAAAAGAATAGAC | |
| | CGAGATAGGGTTGAGTGTTGTTCCAGTTTGGAACAAGA | |
| | GTCCACTATTAAAGAACGTGGACTCCAACGTCAAAGGG | |
| | CGAAAAACCGTCTATCAGGGCGATGGCCCACTACGTGA | |
| | ACCATCACCCTAATCAAGTTTTTTGGGGTCGAGGTGCC | |
| | GTAAAGCACTAAATCGGAACCCTAAAGGGAGCCCCCG | |
| | ATTTAGAGCTTGACGGGGAAAGCCGGCGAACGTGGCG | |
| | AGAAAGGAAGGGAAGAAAGCGAAAGGAGCGGGCGCT | |
| | AGGGCGCTGGCAAGTGTAGCGGTCACGCTGCGCGTAA | |
| | CCACCACACCCGCCGCGCTTAATGCGCCGCTACAGGGC | |
| | GCGTCAGGTGGCACTTTTCGGGGAAATGTGCGCGGAAC | |
| | CCCTATTTGTTTATTTTTCTAAATACATTCAAATATGTA | |
| | TCCGCTCATGAGACAATAACCCTGATAAATGCTTCAAT | |
| | AATATTGAAAAAGGAAGAGTCCTGAGGCGGAAAGAAC | |
| | CAGCTGTGGAATGTGTGTCAGTTAGGGTGTGGAAAGTC | |
| | CCCAGGCTCCCCAGCAGGCAGAAGTATGCAAAGCATG | |
| | CATCTCAATTAGTCAGCAACCAGGTGTGGAAAGTCCCC | |
| | AGGCTCCCCAGCAGGCAGAAGTATGCAAAGCATGCAT | |
| | CTCAATTAGTCAGCAACCATAGTCCCGCCCCTAACTCC | |
| | GCCCATCCCGCCCCTAACTCCGCCCAGTTCCGCCCATTC | |
| | TCCGCCCCATGGCTGACTAATTTTTTTTATTTATGCAGA | |
| | GGCCGAGGCCGCCTCGGCCTCTGAGCTATTCCAGAAGT | |
| | AGTGAGGAGGCTTTTTTGGAGGCCTAGGCTTTTGCAAA | |
| | GATCGATCAAGAGACAGGATGAGGATCGTTTCGCATG | |
| | ATTGAACAAGATGGATTGCACGCAGGTTCTCCGGCCGC | |
| | TTGGGTGGAGAGGCTATTCGGCTATGACTGGGCACAAC | |
| | AGACAATCGGCTGCTCTGATGCCGCCGTGTTCCGGCTG | |
| | TCAGCGCAGGGGCGCCCGGTTCTTTTTGTCAAGACCGA | |
| | CCTGTCCGGTGCCCTGAATGAACTGCAAGACGAGGCAG | |
| | CGCGGCTATCGTGGCTGGCCACGACGGGCGTTCCTTGC | |
| | GCAGCTGTGCTCGACGTTGTCACTGAAGCGGGAAGGG | |
| | ACTGGCTGCTATTGGGCGAAGTGCCGGGGCAGGATCTC | |
| | CTGTCATCTCACCTTGCTCCTGCCGAGAAAGTATCCATC | |
| | ATGGCTGATGCAATGCGGCGGCTGCATACGCTTGATCC | |
| | GGCTACCTGCCCATTCGACCACCAAGCGAAACATCGCA | |
| | TCGAGCGAGCACGTACTCGGATGGAAGCCGGTCTTGTC | |
| | GATCAGGATGATCTGGACGAAGAGCATCAGGGGCTCG | |
| | CGCCAGCCGAACTGTTCGCCAGGCTCAAGGCGAGCATG | |
| | CCCGACGGCGAGGATCTCGTCGTGACCCATGGCGATGC | |
| | CTGCTTGCCGAATATCATGGTGGAAAATGGCCGCTTTT | |
| | CTGGATTCATCGACTGTGGCCGGCTGGGTGTGGCGGAC | |
| | CGCTATCAGGACATAGCGTTGGCTACCCGTGATATTGC | |
| | TGAAGAGCTTGGCGGCGAATGGGCTGACCGCTTCCTCG | |
| | TGCTTTACGGTATCGCCGCTCCCGATTCGCAGCGCATC | |
| | GCCTTCTATCGCCTTCTTGACGAGTTCTTCTGAGCGGGA | |
| | CTCTGGGGTTCGAAATGACCGACCAAGCGACGCCCAAC | |
| | CTGCCATCACGAGATTTCGATTCCACCGCCGCCTTCTAT | |
| | GAAAGGTTGGGCTTCGGAATCGTTTTCCGGGACGCCGG | |
| | CTGGATGATCCTCCAGCGCGGGGATCTCATGCTGGAGT | |
| | TCTTCGCCCACCCTAGGGGGAGGCTAACTGAAACACGG | |
| | AAGGAGACAATACCGGAAGGAACCCGCGCTATGACGG | |
| | CAATAAAAAGACAGAATAAAACGCACGGTGTTGGGTC | |
| | GTTTGTTCATAAACGCGGGGTTCGGTCCCAGGGCTGGC | |
| | ACTCTGTCGATACCCCACCGAGACCCCATTGGGGCCAA | |
| | TACGCCCGCGTTTCTTCCTTTTCCCCACCCCACCCCCCA | |
| | AGTTCGGGTGAAGGCCCAGGGCTCGCAGCCAACGTCG | |
| | GGGCGGCAGGCCCTGCCATAGCCTCAGGTTACTCATAT | |
| | ATACTTTAGATTGATTTAAAACTTCATTTTTAATTTAAA | |
| | AGGATCTAGGTGAAGATCCTTTTTGATAATCTCATGAC | |
| | CAAAATCCCTTAACGTGAGTTTTCGTTCCACTGAGCGT | |
| | CAGACCCCGTAGAAAAGATCAAAGGATCTTCTTGAGAT | |
| | CCTTTTTTTCTGCGCGTAATCTGCTGCTTGCAAACAAAA | |
| | AAACCACCGCTACCAGCGGTGGTTTGTTTGCCGGATCA | |
| | AGAGCTACCAACTCTTTTTCCGAAGGTAACTGGCTTCA | |
| | GCAGAGCGCAGATACCAAATACTGTCCTTCTAGTGTAG | |
| | CCGTAGTTAGGCCACCACTTCAAGAACTCTGTAGCACC | |
| | GCCTACATACCTCGCTCTGCTAATCCTGTTACCAGTGGC | |
| | TGCTGCCAGTGGCGATAAGTCGTGTCTTACCGGGTTGG | |
| | ACTCAAGACGATAGTTACCGGATAAGGCGCAGCGGTC | |
| | GGGCTGAACGGGGGGTTCGTGCACACAGCCCAGCTTG | |
| | GAGCGAACGACCTACACCGAACTGAGATACCTACAGC | |
| | GTGAGCTATGAGAAAGCGCCACGCTTCCCGAAGGGAG | |
| | AAAGGCGGACAGGTATCCGGTAAGCGGCAGGGTCGGA | |
| | ACAGGAGAGCGCACGAGGGAGCTTCCAGGGGGAAACG | |
| | CCTGGTATCTTTATAGTCCTGTCGGGTTTCGCCACCTCT | |
| | GACTTGAGCGTCGATTTTTGTGATGCTCGTCAGGGGGG | |
| | CGGAGCCTATGGAAAAACGCCAGCAACGCGGCCTTTTT | |
| | ACGGTTCCTGGCCTTTTGCTGGCCTTTTGCTCACATGTT | |
| | CTTTCCTGCGTTATCCCCTGATTCTGTGGATAACCGTAT | |
| | TACCGCCATGCAT | |
| RRSP DNA | ATGTTTATCGGTAAGATGCAAGTTGCCCAAGCGGGTAT | 7 |
| | TGATGCAATCGCACAAACAAGACCGGAGCTTGCCGCTC | |
| | GTATCTTTATGGTCGCGATTGAAGAAGCCAACGGTAAA | |
| | CACGTAGGTTTGACGGACATGATGGTTCGTTGGGCCAA | |
| | TGAAGACCCATACTTGGCACCGAAGCATGGTTACAAAG | |
| | GCGAAACGCCAAGTGACCTTGGTTTTGATGCGAAGTAC | |
| | CACGTAGATCTAGGTGAGCATTACGCTGATTTCAAACA | |
| | GTGGTTAGAAACGTCCCAGTCGAACGGGTTGTTGAGTA | |
| | AAGCGACGTTGGATGAATCCACTAAAACGGTTCATCTT | |
| | GGCTATAGCTATCAAGAACTTCAGGATTTGACGGGTGC | |
| | TGAATCGGTGCAAATGGCGTTCTACTTCCTGAAAGAAG | |
| | CGGCGAAGAAAGCGGATCCGATTTCTGGTGATTCAGCT | |
| | GAAATGATACTGCTGAAGAAATTTGCAGATCAAAGCTA | |
| | CTTATCTCAACTTGATTCCGACCGAATGGATCAAATTG | |
| | AAGGTATCTACCGCAGTAGCCATGAGACGGATATTGAC | |
| | GCTTGGGATCGTCGTTACTCTGGTACAGGCTATGATGA | |
| | GCTGACGAATAAGCTTGCTAGTGCAACGGGCGTTGACG | |
| | AGCAGCTTGCGGTTCTTCTGGATGATCGTAAAGGCCTC | |
| | TTGATTGGTGAAGTGCATGGCAGCGACGTCAACGGCCT | |
| | ACGCTTTGTTAATGAACAGATGGATGCACTGAAAAAAC | |
| | AGGGAGTCACAGTCATTGGCCTTGAGCATTTACGCTCA | |
| | GACCTTGCGCAACCGCTGATTGATCGCTACCTAGCTAC | |
| | GGGTGTGATGTCGAGTGAACTAAGCGCAATGCTGAAA | |
| | ACAAAGCATCTCGATGTCACTCTTTTTGAAAACGCACG | |
| | TGCTAACGGTATGCGCATCGTCGCGCTGGATGCAAACA | |
| | GCTCTGCGCGTCCAAATGTTCAGGGAACAGAACATGGT | |
| | CTGATGTACCGTGCTGGTGCTGCGAACAACATTGCGGT | |
| | GGAAGTATTACAAAATCTGCCTGATGGCGAAAAGTTCG | |
| | TTGCTATCTACGGTAAAGCGCATTTGCAGTCTCACAAA | |
| | GGGATTGAAGGGTTCGTTCCTGGTATCACGCACCGTCT | |
| | CGATCTTCCTGCGCTTAAAGTCAGTGACTCGAACCAGT | |
| | TCACAGTTGAACAAGACGATGTAAGTCTACGTGTTGTC | |
| | TACGATGATGTTGCTAACAAACCGAAGATCACGTTCAA | |
| | GGGCAGTTTG | |
| RRSP | QELKERAKVFAKPIGASYQGILDQLDLVHQAKGRDQIAA | 8 |
| Protein | SFELNKKINDYIAEHPTSGRNQALTQLKEQVTSALFIGKM | |
| | QVAQAGIDAIAQTRPELAARIFMVAIEEANGKHVGLTDM | |
| | MVRWANEDPYLAPKHGYKGETPSDLGFDAKYHVDLGEH | |
| | YADFKQWLETSQSNGLLSKATLDESTKTVHLGYSYQELQ | |
| | DLTGAESVQMAFYFLKEAAKKADPISGDSAEMILLKKFA | |
| | DQSYLSQLDSDRMDQIEGIYRSSHETDIDAWDRRYSGTG | |
| | YDELTNKLASATGVDEQLAVLLDDRKGLLIGEVHGSDVN | |
| | GLRFVNEQMDALKKQGVTVIGLEHLRSDLAQPLIDRYLA | |
| | TGVMSSELSAMLKTKHLDVTLFENARANGMRIVALDAN | |
| | SSARPNVQGTEHGLMYRAGAANNIAVEVLQNLPDGEKF | |
| | VAIYGKAHLQSHKGIEGFVPGITHRLDLPALKVSDSNQFT | |
| | VEQDDVSLRVVYDDVANKPKITFKGSL | |

Protein purification of RRSP-DT_{B} and RRSP*-DT_{B} for in vivo experiments was completed as described previously (20). Cell lines were obtained from the National Cancer Institute RAS Initiative, a resource that manages tumor cell lines from collaborators or commercial sources. Ras Initiative cells lines are confirmed free of Mycoplasma using VenorGeM Mycoplasma Classic Endpoint PCR assay and are also subjected to short tandem repeat analysis using the AmpFLSTR Identifiler PCR Amplification Kit to authenticate the cell lines, comparing the results with information located at web.expasy.org/cellosaurus/. Cells were cultured at 37° C. and 5% CO₂ atmosphere. PANC-1 and KPC cells were grown in Dulbecco's Minimal Eagle's Medium (DMEM) (ATCC formulation) with 10% fetal bovine serum (FBS) and 1% penicillin-streptomycin (P/S) and HCT-116 cells were grown in DMEM-F12 with Glutamax (Gibco) containing 10% FBS and 1% P/S.

### Nanoparticle Formulation and Characterization

Briefly, block copolymers, PEG₁₇-b-PPS₈₀-pyridyl disulfide were synthesized as previously described and conjugated to the cationic dendritic peptide (DP) via disulfide exchange as previously described (24). GMP grade synthesis of PEG₁₇-b-PPS₈₀-pyridyl disulfide was performed in collaboration with our contract research organization Sequens Group, while the final DP-end capping was completed in-house within a clean room. The resulting sterile PPDP2 was mixed with rrsp mRNA or CleanCap® mCherry-mRNA (TriLink BioTechnologies) in 25 mM sodium acetate buffer at the weight ratio of 40:1 to assemble monodisperse spherical complexes for mRNA transfection or fluorescent tracing of cell transfection, respectively. For quality control, PPDP2-rrsp-mRNA and PPDP2-mCherry-mRNA were assessed by dynamic light scattering, NanoSight, cryogenic electron microscopy, and high-performance liquid chromatography to ensure consistent nanoparticle diameter, concentration, structure, and nucleic acid loading efficiency, respectively, as previously described (24)

### Epifluorescence

KPC and PANC-1 cells were plated on 4-well slides in DMEM (10% FBS, 1% P/S) media and allowed to attach overnight. The following day, 1 μg GFP-mRNA was mixed with 40 μg of PPDP2 in ddH₂O for 30 minutes at room temperature or 1.5 μL MessengerMax Lipofectamine (Invitrogen) as a positive control according to manufacturer's instructions. The mixture was added to cells along with negative controls, gfp-mRNA and nanoparticles alone. After 24-hour incubation, cells were washed three times for 5 minutes with phosphate-buffered saline (PBS), fixed with 100% methanol for 10 minutes, washed three times for 5 minutes with PBS, and mounted with 4′,6-diamidino-2-phenylindole (DAPI) stain. Cells were visualized on a Nikon Ti2 widefield microscope using a 40× objective. Five images were taken for each treatment and the total number of cells were counted per frame and compared to the number of cells that were GFP positive.

### SDS-PAGE and Western Blotting

Protein extracts were prepared by either directly adding 2×SDS buffer to the well or by harvesting cells by adding radioimmunoprecipitation assay buffer (50 mM Tris-HCl, pH 7.4, 150 mM NaCl, 2 mM EDTA, 1% Nonidet P-40, 1% Na-deoxycholate, 0.1% SDS), supplemented with 1× protease and phosphatase inhibitors and 1 mM phenylmethylsulfonyl fluoride (PMSF). Equal amounts of proteins or equal volumes were separated by SDS-PAGE followed by Western blot analysis as described previously (24). Membranes were blotted using the following antibodies: anti-pan-RAS (Thermo Fisher Scientific, catalog no. MA1-012, RRID:AB_2536664), which recognizes RAS Switch I and thus detects only uncleaved RAS. Anti-Phospho-p44/42 MAPK (pERK1/2; Cell Signaling Technology, catalog no. 4377, RRID:AB_331775), anti-p44/42 MAPK (ERK1/2; Cell Signaling Technology, catalog no. 4696, RRID:AB_390780), and anti-HB-EGF (Abcam, #ab185555). Anti-vinculin (Cell Signaling Technology, catalog no. 13901, RRID:AB_2728768) or anti-GAPDH (Cell Signaling Technology, catalog no. 2118S) (as indicated) was used for normalization. Secondary antibodies used were fluorescent-labeled IRDye 680RD goat anti-mouse (LI-COR Biosciences, catalog no. 926-68070, RRID:AB_10956588) and IRDye 800CW goat anti-rabbit (LI-COR Biosciences, catalog no. 926-32211, RRID:AB_621843). Blot images were acquired using the Odyssey Infrared Imaging System (LI-COR Biosciences) and quantified by densitometry using NIH ImageJ software v2.14.0/1.54f (ImageJ, RRID:SCR_003070). Percentage of uncleaved RAS and pERK/ERK was calculated as described previously (21).

Protein extracts from frozen tissues were prepared by pulverizing tissue with mortar and pestle and homogenizing tissue in a microcentrifuge tube containing RIPA buffer (50 mM Tris-HCl, Ph 7.4, 150 mM NaCl, 2 mM EDTA, 1% Nonidet P-40, 1% Na-deoxycholate, 0.1% SDS), supplemented with 1× protease and phosphatase inhibitors and 1 mM PMSF. Samples were homogenized on ice three times, 5 seconds each time, incubated on ice for 30 minutes, and centrifuged at 13,500 rpm for 15 minutes at 4° C. Supernatant fluid was collected and protein content measured using the bicinchoninic acid protein assay kit (Thermo Fisher Scientific) according to manufacturer's instructions.

### Cytotoxicity and Apoptosis Assays

Apoptosis was assessed by Caspase-Glo 3/7 assay (Promega) according to manufacturer's instructions. A total of 10,000 cells/well were grown in 24-well clear plates and treated with 5.5 μg (in vivo concentration) rrsp-mRNA or rrsp*-mRNA by PPDP2 nanoparticles for 24 hours. Caspase-Glo 3/7 was then added to each well and luminescence was recorded using a Tecan Safire2 plate reader at 570 nM.

Cytotoxicity was assessed by staining cells with crystal violet, as previously described (20). Briefly, 2×10⁴ cells/well were cultured in 24-well plates and treated with either 0.25, 0.5, 1, 1.5 or 2 μg rrsp mRNA by PPDP2 nanoparticles or 2 μg rrsp mRNA via Lipofectamine MessengerMAX (Invitrogen) for 72 hours. Cells were washed and crystal violet fixing/staining solution was added for 20 minutes at room temperature as described previously (20). Images of air-dried plates were acquired using a conventional desktop scanner followed by dissolving crystal violet in methanol. Absorbance was read at 570 nm for quantitative analysis. at 570 nM.

For HCT-116 cells, cells were seeded into 12 well plate at ˜1×10⁵ cells per well overnight (˜80% confluency). Cells were transfected with EGFP-mRNA (8 μg) or rrsp-mRNA (8 μg, 4 μg, 1 μg, 0.5 μg) using Lipofectamine MessengerMax according to manufacturer's protocol. Images were taken using EVOS microscope at indicted timepoints. Lysates were collected after 24 hours and probed for RAS as described previously (19). For KPC cells, ˜6.5×10⁶ cells were seeded in a 12 well plate until cells were attached (˜2 hours). Once cells were attached, treatment with rrsp mRNA was transfected into KPC cells using Lipofectamine MessengerMax. Cells were imaged over time using Biostation CT (Nikon Instruments). Calculation of percent confluency was done using Nikon Elements v4.11.0 as described previously (19).

### In Vivo MTD in DTR Mice

In collaboration with Jackson Laboratory (Bar Harbor, ME), we created a custom DTR mouse model, expressing the DT receptor human HB-EGF in all mouse cells by crossing the ROSA26iDTR mouse strain with the B6.FVB-Tg (EIIa-cre) C5379Lmgd/J strain to create DTR Knock-In mice. We divided the mice into 7 treatment groups with 5 mice per treatment group and 4 mice in the control group. Treatments included 0.05 mg/kg RRSP-DT_{B}, 0.1 mg/kg RRSP-DT_{B}, 0.25 mg/kg RRSP-DT_{B}, 0.5 mg/kg of RRSP-DT_{B}, or 0.25 and 0.5 mg/kg of catalytically inactive RRSP*-DT_{B}. Mice were treated every other day for 4 weeks, excluding weekends, weighed, and assessed for signs of distress once per day. Median weights across all groups were recorded along with occurrence of deaths. At the end of the experiment, tissue was harvested from heart, kidney, liver, lungs, and spleen from control group and highest dosed group to observe off-target effects.

### Flow Cytometry for DTR Expression

PBMCs were harvested from mouse blood from DTR Knock-In and DTR WT mice via density gradient centrifugation over Ficoll and PBMCs were cultured for use in Western blotting. Cells were then stained with anti-HB-EGF as per manufacturer's instructions and expression of HB-EGF was assessed by flow cytometry using the BD-FACSCelesta.

### In Vivo Tumors

When tumors reached an average size of 80-120 mm³, mice were randomized into groups of 5 and treatment started. The first group received PPDP2 (25 mM sodium acetate buffer), the second 0.25 mg/kg of PPDP2+rrsp mRNA (1×, every other day), the third 0.25 mg/kg of PPDP2+rrsp*mRNA (1×, every other day), the fourth PPDP2+mCherry mRNA. Both tumor size and mouse body weight were measured every other day. At the end of the treatment schedule, mice were euthanized, tumors excised, and either snap frozen in liquid N₂ or fixed in 10% formalin overnight. A repeat experiment was conducted identically except mice were grafted with two tumors, one on each flank, and only one tumor was treated.

### Histology, IHC, and Image Analysis

Paraffin-embedding, sectioning, hematoxylin and eosin (H&E) and IHC staining of mouse tissue specimens were performed by the Robert H. Lurie Comprehensive Mouse Histology and Pathology Core Facility. Tumor sections were stained with anti-cytokeratin 19 [(CK-19), #ab76539; Abcam], anti-Ki-67 (#GA626; Dako), anti-pan-RAS [(RAS), #PA5-85947; Thermo Fisher Scientific], and anti-Phospho-p44/42 MAPK [(ERK1/2; Thr202/Tyr204, (D13.14.4E) XP, #4370; Cell Signaling Technology] antibodies as described previously (20). Primary antibodies were detected using the appropriate secondary antibodies and 3,3⁰-diaminobenzidine revelation (Dako).

### Statistical Analysis

Graphpad Prism v.10 software was used for statistical analysis. Bar plots represent the mean of at least three independent experiments and the SD or SEM as indicated in Figure legends. Statistical significance was assessed using one-way ANOVA assuming normal distribution. Dunnett multiple comparison post-test was employed to compare the mean of the control group with the mean of treatment groups. Tukey's multiple comparison test was used to compare the mean of each group with the mean of every other group. Values of p<0.05 were considered statistically significant. Pairwise tests were analyzed using Student's t-test.

### Structure Modeling and Analysis

The multimeric structure of V. vulnificus RRSP bound to human KRAS was modeled using ColabFold v1.5.1 (Mirdita et al (2022) Nat Methods 19:679-682)) on the Google Colab virtual machine via the interface embedded within UCSF ChimeraX structure analysis software (Meng et al (2023) Protein Sci 32: e4792). All structures were visualized, aligned, and analyzed in ChimeraX. The resulting RRSP model has r.m.s.d.=0692 with the published RRSP structure (PDB ID 5w6l, www.rcsb.org) and the resulting KRAS structure has r.m.s.d=0.629 with the published KRAS structure (PDB ID 4obe, www.rcsb.org). Close contacts was determined using the resulting model analyzed using the MAPIYA contact map server (mapiya.icbio.pl) (Badaczewska-Dawid et al (2022) Nucleic Acids Res 50: W474-W482).

### Example 2-(PPDP2) Nanoparticles for Delivery of an RRSP DNA to Cells

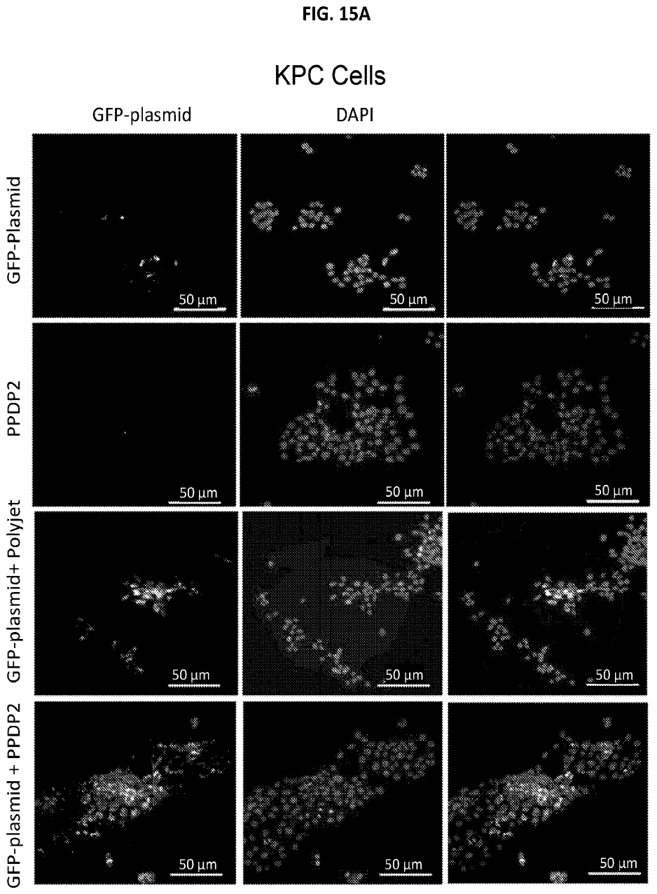

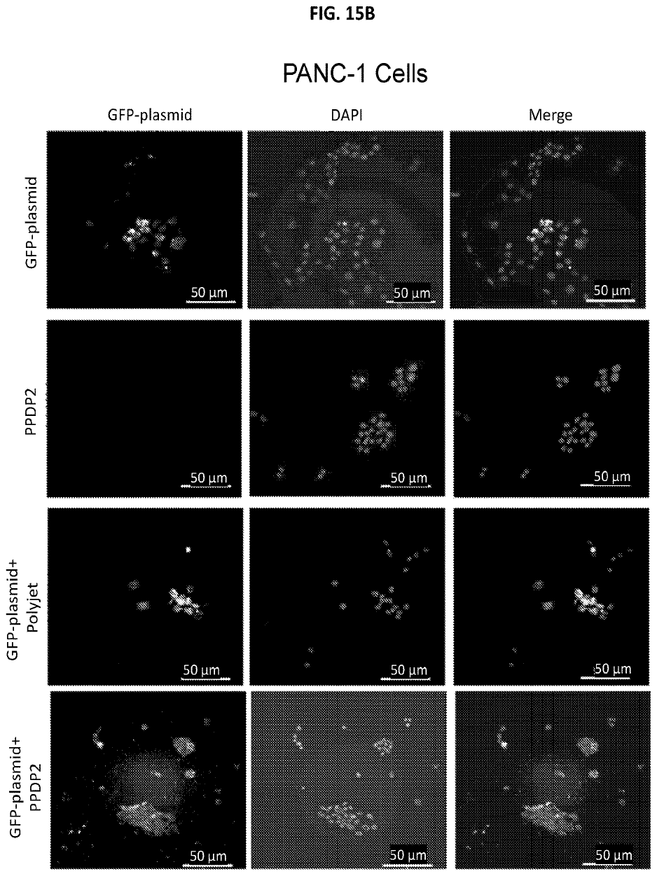

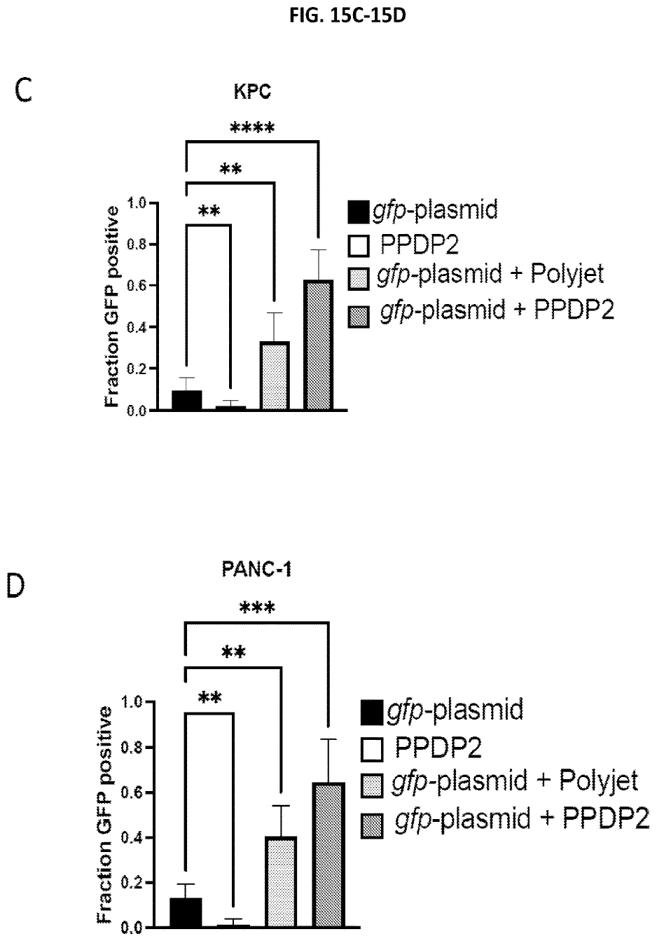

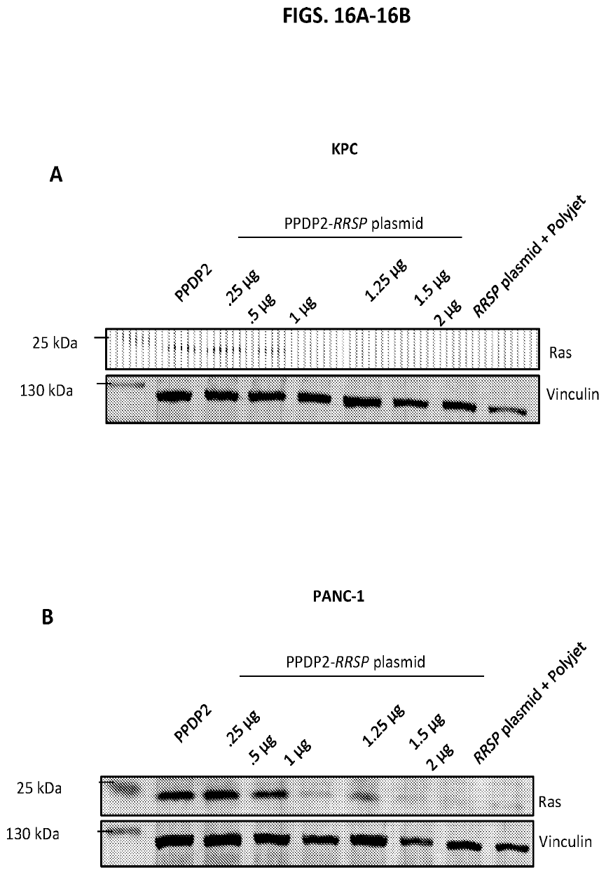

7.5×10⁴ mouse pancreatic KPC (FIG. 15A) and human pancreatic PANC-1 cells (FIG. 15B) were plated in 24-well plates and allowed to attach overnight. The following day, the indicated amount of pEGFP-N3 (Clontech) or RRSP-plasmid (SEQ ID NO: 6) prepared by the alkaline lysis method was used to transfect cells. Plasmid DNA was pre-mixed with PPDP2 (1:40 ratio) and then added to cells for 24 hours with either PPDP2 nanoparticles alone, PPDP2 nanoparticles loaded with either 0.25, 0.5, 1, 1.25, 1.5 or 2 μg RRSP plasmid (SEQ ID NO: 6) or 2 μg RRSP-plasmid mixed with 2 μl PolyJet reagent (SignaGen). For epifluorescence, after 24-hour incubation, cells were washed three times for 5 minutes with phosphate-buffered saline (PBS), fixed with 100% methanol for 10 minutes, washed three times for 5 minutes with PBS, and mounted with 4′,6-diamidino-2-phenylindole (DAPI) stain. Cells were visualized on a Nikon Ti2 widefield microscope using a 40× objective. Five images were taken for each treatment and the total number of cells were counted per frame and compared to the number of cells that were GFP positive. For Western blot, cell lysate was harvested the following day by direct application of 2× SDS buffer and boiled for 10 minutes. Western blot samples were equal volume loaded.

Plasmid DUFVvC2-EGFP from Antic, Biancucci, Satchell (2014) Proteins 82:2643-2656 [16] Sequence: RRSP sequence underlined; GFP sequence underlined; vector pEGFP-N3 (Clontech) NheI (GCGAGC) and SalI (GTCGAC) sites used for cloning indicated by boxes.

(SEQ ID NO: 6)
GTACATGACCTTATGGGACTTTCCTACTTGGCAGTACATCTACGTATTAGTCATCGCT
ATTACCATGGTGATGCGGTTTTGGCAGTACATCAATGGGCGTGGATAGCGGTTTGAC
TCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAAT
GGGCGGTAGGCGTGTACGGTGGGAGGTCTATATAAGCAGAGCTGGTTTAGTGAACC

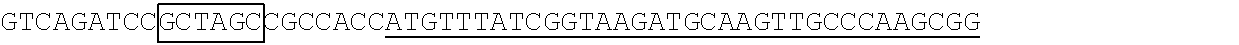

GTATTGATGCAATCGCACAAACAAGACCGGAGCTTGCCGCTCGTATCTTTATGGTCG
CGATTGAAGAAGCCAACGGTAAACACGTAGGTTTGACGGACATGATGGTTCGTTGG
GCCAATGAAGACCCATACTTGGCACCGAAGCATGGTTACAAAGGCGAAACGCCAAG
TGACCTTGGTTTTGATGCGAAGTACCACGTAGATCTAGGTGAGCATTACGCTGATTT
CAAACAGTGGTTAGAAACGTCCCAGTCGAACGGGTTGTTGAGTAAAGCGACGTTGG
ATGAATCCACTAAAACGGTTCATCTTGGCTATAGCTATCAAGAACTTCAGGATTTGA
CGGGTGCTGAATCGGTGCAAATGGCGTTCTACTTCCTGAAAGAAGCGGCGAAGAAA
GCGGATCCGATTTCTGGTGATTCAGCTGAAATGATACTGCTGAAGAAATTTGCAGAT
CAAAGCTACTTATCTCAACTTGATTCCGACCGAATGGATCAAATTGAAGGTATCTAC
CGCAGTAGCCATGAGACGGATATTGACGCTTGGGATCGTCGTTACTCTGGTACAGGC
TATGATGAGCTGACGAATAAGCTTGCTAGTGCAACGGGCGTTGACGAGCAGCTTGC
GGTTCTTCTGGATGATCGTAAAGGCCTCTTGATTGGTGAAGTGCATGGCAGCGACGT
CAACGGCCTACGCTTTGTTAATGAACAGATGGATGCACTGAAAAAACAGGGAGTCA
CAGTCATTGGCCTTGAGCATTTACGCTCAGACCTTGCGCAACCGCTGATTGATCGCT
ACCTAGCTACGGGTGTGATGTCGAGTGAACTAAGCGCAATGCTGAAAACAAAGCAT
CTCGATGTCACTCTTTTTGAAAACGCACGTGCTAACGGTATGCGCATCGTCGCGCTG
GATGCAAACAGCTCTGCGCGTCCAAATGTTCAGGGAACAGAACATGGTCTGATGTA
CCGTGCTGGTGCTGCGAACAACATTGCGGTGGAAGTATTACAAAATCTGCCTGATGG
CGAAAAGTTCGTTGCTATCTACGGTAAAGCGCATTTGCAGTCTCACAAAGGGATTGA
AGGGTTCGTTCCTGGTATCACGCACCGTCTCGATCTTCCTGCGCTTAAAGTCAGTGA
CTCGAACCAGTTCACAGTTGAACAAGACGATGTAAGTCTACGTGTTGTCTACGATGA

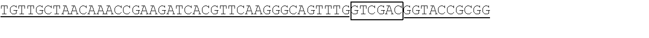

GCCCGGGATCCATCGCCACCATGGTGAGCAAGGGCGAGGAGCTGTTCACCGGGGTG
GTGCCCATCCTGGTCGAGCTGGACGGCGACGTAAACGGCCACAAGTTCAGCGTGTC
CGGCGAGGGCGAGGGCGATGCCACCTACGGCAAGCTGACCCTGAAGTTCATCTGCA
CCACCGGCAAGCTGCCCGTGCCCTGGCCCACCCTCGTGACCACCCTGACCTACGGCG
TGCAGTGCTTCAGCCGCTACCCCGACCACATGAAGCAGCACGACTTCTTCAAGTCCG
CCATGCCCGAAGGCTACGTCCAGGAGCGCACCATCTTCTTCAAGGACGACGGCAAC
TACAAGACCCGCGCCGAGGTGAAGTTCGAGGGCGACACCCTGGTGAACCGCATCGA
GCTGAAGGGCATCGACTTCAAGGAGGACGGCAACATCCTGGGGCACAAGCTGGAGT
ACAACTACAACAGCCACAACGTCTATATCATGGCCGACAAGCAGAAGAACGGCATC
AAGGTGAACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAACC
ACTACCTGAGCACCCAGTCCGCCCTGAGCAAAGACCCCAACGAGAAGCGCGATCAC
ATGGTCCTGCTGGAGTTCGTGACCGCCGCCGGGATCACTCTCGGCATGGACGAGCTG
TACAAGTAAAGCGGCCGCGACTCTAGATCATAATCAGCCATACCACATTTGTAGAG
GTTTTACTTGCTTTAAAAAACCTCCCACACCTCCCCCTGAACCTGAAACATAAAATG
AATGCAATTGTTGTTGTTAACTTGTTTATTGCAGCTTATAATGGTTACAAATAAAGCA
ATAGCATCACAAATTTCACAAATAAAGCATTTTTTTCACTGCATTCTAGTTGTGGTTT
GTCCAAACTCATCAATGTATCTTAAGGCGTAAATTGTAAGCGTTAATATTTTGTTAA
AATTCGCGTTAAATTTTTGTTAAATCAGCTCATTTTTTAACCAATAGGCCGAAATCGG
CAAAATCCCTTATAAATCAAAAGAATAGACCGAGATAGGGTTGAGTGTTGTTCCAGT
TTGGAACAAGAGTCCACTATTAAAGAACGTGGACTCCAACGTCAAAGGGCGAAAAA
CCGTCTATCAGGGCGATGGCCCACTACGTGAACCATCACCCTAATCAAGTTTTTTGG
GGTCGAGGTGCCGTAAAGCACTAAATCGGAACCCTAAAGGGAGCCCCCGATTTAGA
GCTTGACGGGGAAAGCCGGCGAACGTGGCGAGAAAGGAAGGGAAGAAAGCGAAAG
GAGCGGGCGCTAGGGCGCTGGCAAGTGTAGCGGTCACGCTGCGCGTAACCACCACA
CCCGCCGCGCTTAATGCGCCGCTACAGGGCGCGTCAGGTGGCACTTTTCGGGGAAAT
GTGCGCGGAACCCCTATTTGTTTATTTTTCTAAATACATTCAAATATGTATCCGCTCA
TGAGACAATAACCCTGATAAATGCTTCAATAATATTGAAAAAGGAAGAGTCCTGAG
GCGGAAAGAACCAGCTGTGGAATGTGTGTCAGTTAGGGTGTGGAAAGTCCCCAGGC
TCCCCAGCAGGCAGAAGTATGCAAAGCATGCATCTCAATTAGTCAGCAACCAGGTG
TGGAAAGTCCCCAGGCTCCCCAGCAGGCAGAAGTATGCAAAGCATGCATCTCAATT
AGTCAGCAACCATAGTCCCGCCCCTAACTCCGCCCATCCCGCCCCTAACTCCGCCCA
GTTCCGCCCATTCTCCGCCCCATGGCTGACTAATTTTTTTTATTTATGCAGAGGCCGA
GGCCGCCTCGGCCTCTGAGCTATTCCAGAAGTAGTGAGGAGGCTTTTTTGGAGGCCT
AGGCTTTTGCAAAGATCGATCAAGAGACAGGATGAGGATCGTTTCGCATGATTGAA
CAAGATGGATTGCACGCAGGTTCTCCGGCCGCTTGGGTGGAGAGGCTATTCGGCTAT
GACTGGGCACAACAGACAATCGGCTGCTCTGATGCCGCCGTGTTCCGGCTGTCAGCG
CAGGGGCGCCCGGTTCTTTTTGTCAAGACCGACCTGTCCGGTGCCCTGAATGAACTG
CAAGACGAGGCAGCGCGGCTATCGTGGCTGGCCACGACGGGCGTTCCTTGCGCAGC
TGTGCTCGACGTTGTCACTGAAGCGGGAAGGGACTGGCTGCTATTGGGCGAAGTGC
CGGGGCAGGATCTCCTGTCATCTCACCTTGCTCCTGCCGAGAAAGTATCCATCATGG
CTGATGCAATGCGGCGGCTGCATACGCTTGATCCGGCTACCTGCCCATTCGACCACC
AAGCGAAACATCGCATCGAGCGAGCACGTACTCGGATGGAAGCCGGTCTTGTCGAT
CAGGATGATCTGGACGAAGAGCATCAGGGGCTCGCGCCAGCCGAACTGTTCGCCAG
GCTCAAGGCGAGCATGCCCGACGGCGAGGATCTCGTCGTGACCCATGGCGATGCCT
GCTTGCCGAATATCATGGTGGAAAATGGCCGCTTTTCTGGATTCATCGACTGTGGCC
GGCTGGGTGTGGCGGACCGCTATCAGGACATAGCGTTGGCTACCCGTGATATTGCTG
AAGAGCTTGGCGGCGAATGGGCTGACCGCTTCCTCGTGCTTTACGGTATCGCCGCTC
CCGATTCGCAGCGCATCGCCTTCTATCGCCTTCTTGACGAGTTCTTCTGAGCGGGACT
CTGGGGTTCGAAATGACCGACCAAGCGACGCCCAACCTGCCATCACGAGATTTCGA
TTCCACCGCCGCCTTCTATGAAAGGTTGGGCTTCGGAATCGTTTTCCGGGACGCCGG
CTGGATGATCCTCCAGCGCGGGGATCTCATGCTGGAGTTCTTCGCCCACCCTAGGGG
GAGGCTAACTGAAACACGGAAGGAGACAATACCGGAAGGAACCCGCGCTATGACG
GCAATAAAAAGACAGAATAAAACGCACGGTGTTGGGTCGTTTGTTCATAAACGCGG
GGTTCGGTCCCAGGGCTGGCACTCTGTCGATACCCCACCGAGACCCCATTGGGGCCA
ATACGCCCGCGTTTCTTCCTTTTCCCCACCCCACCCCCCAAGTTCGGGTGAAGGCCC
AGGGCTCGCAGCCAACGTCGGGGCGGCAGGCCCTGCCATAGCCTCAGGTTACTCAT
ATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCG
TCAGACCCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTTGCCGGAT
CAAGAGCTACCAACTCTTTTTCCGAAGGTAACTGGCTTCAGCAGAGCGCAGATACCA
AATACTGTCCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGG
AGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAAA
ACGCCAGCAACGCGGCCTTTTTACGGTTCCTGGCCTTTTGCTGGCCTTTTGCTCACAT
GTTCTTTCCTGCGTTATCCCCTGATTCTGTGGATAACCGTATTACCGCCATGCAT

### REFERENCES

1 Siegel R L, Miller K D, Wagle N S, Jemal A. Cancer statistics, 2023. C A: A Cancer Journal for Clinicians. 2023; 73(1):17-48.
2. Pezzilli R, Fabbri D, Imbrogno A. Pancreatic ductal adenocarcinoma screening: new perspectives. World J Gastroenterol. 2012; 18(36):4973-7.
3. Hezel A F, Kimmelman A C, Stanger B Z, Bardeesy N, Depinho R A. Genetics and biology of pancreatic ductal adenocarcinoma. Genes Dev. 2006; 20(10):1218-49.
4. McCormick F. Progress in targeting RAS with small molecule drugs. Biochem J. 2019; 476(2):365-74.
5. McGee J H, Shim S Y, Lee S J, Swanson P K, Jiang S Y, Durney M A, et al. Exceptionally high-affinity Ras binders that remodel its effector domain. J Biol Chem. 2018; 293(9):3265-80.
6. Martin-Acosta P, Xiao X. PROTACs to address the challenges facing small molecule inhibitors. Eur J Med Chem. 2021; 210:112993.
7 Xi M, Chen Y, Yang H, Xu H, Du K, Wu C, et al. Small molecule PROTACs in targeted therapy: An emerging strategy to induce protein degradation. Eur J Med Chem. 2019; 174:159-80.
8. Yao T, Xiao H, Wang H, Xu X. Recent Advances in PROTACs for Drug Targeted Protein Research. Int J Mol Sci. 2022; 23(18).
9 Amgen. Expanded Access of Sotorasib. ClinicalTrials.gov/show/NCT04667234.
10. Expanded Access of Adagrasib (MRTX849) in Patients With Advanced Solid Tumors Who Have a KRAS G12C Mutation. ClinicalTrials.gov/show/NCT05162443.
11. Escher T E, Satchell K J F. RAS Degraders: The New Frontier for RAS Driven Cancers. Molecular Therapy. 2023.
12. Bobbala S, Allen S D, Yi S, Vincent M, Frey M, Karabin N B, et al. Employing bicontinuous-to-micellar transitions in nanostructure morphology for on-demand photo-oxidation responsive cytosolic delivery and off-on cytotoxicity. Nanoscale. 2020; 12(9):5332-40.
13. Burke J A, Zhang X, Bobbala S, Frey M A, Bohorquez Fuentes C, Freire Haddad H, et al. Subcutaneous nanotherapy repurposes the immunosuppressive mechanism of rapamycin to enhance allogeneic islet graft viability. Nat Nanotechnol. 2022; 17(3):319-30.
14. Dowling D J, Scott E A, Scheid A, Bergelson I, Joshi S, Pietrasanta C, et al. Toll-like receptor 8 agonist nanoparticles mimic immunomodulating effects of the live BCG vaccine and enhance neonatal innate and adaptive immune responses. J Allergy Clin Immunol. 2017; 140(5):1339-50.
15. Hyun S, Shin D. Small-Molecule Inhibitors and Degraders Targeting KRAS-Driven Cancers. Int J Mol Sci. 2021; 22(22).
16. Antic I, Biancucci M, Satchell K J. Cytotoxicity of the Vibrio vulnificus MARTX toxin effector DUF5 is linked to the C2A subdomain. Proteins. 2014; 82(10):2643-56.
17. Antic I, Biancucci M, Zhu Y, Gius D R, Satchell K J F. Site-specific processing of Ras and Rap1 Switch I by a MARTX toxin effector domain. Nat Commun. 2015; 6:7396.
18. Biancucci M, Rabideau A E, Lu Z, Loftis A R, Pentelute B L, Satchell K J F. Substrate Recognition of MARTX Ras/Rap1-Specific Endopeptidase. Biochemistry. 2017; 56(21):2747-57.
19. Stubbs C K, Biancucci M, Vidimar V, Satchell K J F. RAS specific protease induces irreversible growth arrest via p27 in several KRAS mutant colorectal cancer cell lines. Sci Rep. 2021; 11(1):17925.
20. Vidimar V, Park M, Stubbs C K, Ingram N K, Qiang W, Zhang S, et al. Proteolytic pan-RAS Cleavage Leads to Tumor Regression in Patient-derived Pancreatic Cancer Xenografts. Mol Cancer Ther. 2022; 21(5):810-20.
21. Vidimar V, Beilhartz G L, Park M, Biancucci M, Kieffer M B, Gius D R, et al. An engineered chimeric toxin that cleaves activated mutant and wild-type RAS inhibits tumor growth. Proc Natl Acad Sci USA. 2020; 117(29):16938-48.
22. Biancucci M, Minasov G, Banerjee A, Herrera A, Woida P J, Kieffer M B, et al. The bacterial Ras/Rap1 site-specific endopeptidase RRSP cleaves Ras through an atypical mechanism to disrupt Ras-ERK signaling. Sci Signal. 2018; 11(550).
23. Yi S, Allen S D, Liu Y G, Ouyang B Z, Li X, Augsornworawat P, et al. Tailoring Nanostructure Morphology for Enhanced Targeting of Dendritic Cells in Atherosclerosis. ACS Nano. 2016; 10(12):11290-303.
24. Yi S, Kim S Y, Vincent M P, Yuk S A, Bobbala S, Du F, et al. Dendritic peptide-conjugated polymeric nanovectors for non-toxic delivery of plasmid DNA and enhanced non-viral transfection of immune cells. iScience. 2022; 25(7):104555.
25. Cai W, Luo T, Chen X, Mao L, Wang M. A Combinatorial Library of Biodegradable Lipid Nanoparticles Preferentially Deliver mRNA into Tumor Cells to Block Mutant RAS Signaling. Advanced Functional Materials.n/a(n/a):2204947.
26. Allen S, Vincent M, Scott E. Rapid, Scalable Assembly and Loading of Bioactive Proteins and Immunostimulants into Diverse Synthetic Nanocarriers Via Flash Nanoprecipitation. J Vis Exp. 2018(138).
27. Gill D M. Bacterial toxins: a table of lethal amounts. Microbiol Rev. 1982; 46(1):86-94.
28. Ebi YAaH. National Cancer Institute: RAS Dialogue Blog 2023.
29. Bernard V, Fleming J, Maitra A. Molecular and Genetic Basis of Pancreatic Carcinogenesis: Which Concepts May be Clinically Relevant? Surg Oncol Clin N Am. 2016; 25(2):227-38.
30. A Phase 1/2, Study Evaluating the Safety, Tolerability, PK, and Efficacy of Sotorasib (AMG 510) in Subjects With Solid Tumors With a Specific KRAS Mutation (CodeBreak 100).
31. Nagashima T, Inamura K, Nishizono Y, Suzuki A, Tanaka H, Yoshinari T, et al. ASP3082, a First-in-class novel KRAS G12D degrader, exhibits remarkable anti-tumor activity in KRAS G12D mutated cancer models. European Journal of Cancer. 2022; 174: S30.
32. Wang X, Shen J, Jiang F, Jin Q. The Photorhabdus Virulence Cassettes RRSP-Like Effector Interacts With Cyclin-Dependent Kinase 1 and Causes Mitotic Defects in Mammalian Cells. Frontiers in Microbiology. 2020; 11.
33. Janne P A. Progress in the treatment of KRAS mutant cancers. American Association for Cancer Research Annual Meeting 2023, Orlando, FL. 2023.
34. Schulze C J, Seamon K J, Zhao Y, Yang Y C, Cregg J, Kim D, et al. Chemical remodeling of a cellular chaperone to target the active state of mutant KRAS. Science. 2023; 381(6659):794-9.
35. Zhao Y, Adjei A A. The clinical development of MEK inhibitors. Nature Reviews Clinical Oncology. 2014; 11(7):385-400.
36. Welsh S J, Corrie P G. Management of BRAF and MEK inhibitor toxicities in patients with metastatic melanoma. Therapeutic Advances in Medical Oncology. 2015; 7(2):122-36.
37. Sunayama J, Matsuda K, Sato A, Tachibana K, Suzuki K, Narita Y, et al. Crosstalk between the PI3K/mTOR and MEK/ERK pathways involved in the maintenance of self-renewal and tumorigenicity of glioblastoma stem-like cells. Stem Cells. 2010; 28(11):1930-9.
38. Zugasti O, Rul W, Roux P, Peyssonnaux C, Eychene A, Franke T F, et al. Raf-MEK-Erk cascade in anoikis is controlled by Rac1 and Cdc42 via Akt. Mol Cell Biol. 2001; 21(19):6706-17.
39. Awad M M, Liu S, Rybkin I I, Arbour K C, Dilly J, Zhu V W, et al. Acquired Resistance to KRASG12C Inhibition in Cancer. New England Journal of Medicine. 2021; 384(25):2382-93.
40. Moore A R, Rosenberg S C, McCormick F, Malek S. Author Correction: RAS-targeted therapies: is the undruggable drugged? Nat Rev Drug Discov. 2020; 19(12):902.
41. Hobbs G A, Der C J, Rossman K L. RAS isoforms and mutations in cancer at a glance. J Cell Sci. 2016; 129(7):1287-92.
42. Kapoor A, Yao W, Ying H, Hua S, Liewen A, Wang Q, et al. Yap1 Activation Enables Bypass of Oncogenic Kras Addiction in Pancreatic Cancer. Cell. 2014; 158(1):185-97.
43. Yuan T L, Amzallag A, Bagni R, Yi M, Afghani S, Burgan W, et al. Differential Effector Engagement by Oncogenic KRAS. Cell Reports. 2018; 22(7):1889-902.
44. Quandt J, Schlude C, Bartoschek M, Will R, Cid-Arregui A, Scholch S, et al. Long-peptide vaccination with driver gene mutations in p53 and Kras induces cancer mutation-specific effector as well as regulatory T cell responses. Oncoimmunology. 2018; 7(12): e1500671.
45. Ross, S. J., Revenko, A. S., Hanson, L. L., Ellston, R., Staniszewska, A., Whalley, N., Pandey, S. K., Revill, M., Rooney, C., Buckett, L. K., et al. (2017). Targeting KRAS-dependent tumors with AZD4785, a high-affinity therapeutic antisense oligonucleotide inhibitor of KRAS. Sci Transl Med. 9(394).
46. Zeng, M., Lu, J., Li, L., Feru, F., Quan, C., Gero, T. W., Ficarro, S. B., Xiong, Y., Ambrogio, C., Paranal, R. M., et al. (2017). Potent and Selective Covalent Quinazoline Inhibitors of KRAS G12C. Cell Chem Biol. 24(8), 1005-1016 e1003.
47. Loftis, A. R., Santos, M. S., Truex, N. L., Biancucci, M., Satchell, K. J. F., and Pentelute, B. L. (2020). Anthrax Protective Antigen Retargeted with Single-Chain Variable Fragments Delivers Enzymes to Pancreatic Cancer Cells. Chembiochem. 21(19), 2772-2776.

1. A system for delivery of a nucleic acid encoding RAS/RAP1-specific endopeptidase (RRSP) to a cell, the system comprising:
a nanocarrier comprising a poly(ethylene glycol)-block-poly(propylene sulfide) copolymer (PEG-b-PPS) conjugated with a dendritic-specific branched cationic peptide (DP) (PPDP); and
a nucleic acid encoding RRSP.

2. The system of claim 1, wherein the nucleic acid is an mRNA comprising a sequence having at least 90% identity to SEQ ID NO: 4.

3. The system of claim 1, wherein the RRSP is a catalytically inactive form of RRSP.

4. The system of claim 3, wherein the nucleic acid is an mRNA comprising a sequence having at least 90% identity to SEQ ID NO: 3

5. The system of claim 1, wherein the nucleic acid is a DNA comprising a sequence having at least 90% identity to SEQ ID NO: 7.

6. The system of claim 1, wherein the PEG-b-PPS has a PEG weight fraction of between about 0.05 and about 0.50.

7. The system of claim 1, wherein the PEG-b-PPS is PEG₁₇-b-PPS₈₀.

8. The system of claim 1 wherein the mass ratio (w/w) of PPDP:nucleic acid is between about 15:1 and about 120:1.

9. A pharmaceutical composition comprising the system of claim 1.

10. A method of delivering a nucleic acid encoding RRSP to a cell, the method comprising contacting the cell with the system of claim 1.

11. The method of claim 10, wherein the cell is a mammalian cell.

12. The method of claim 10, wherein the cell is a non-phagocytic cell.

13. The method of claim 10, wherein the cell is a cancer cell.

14. The method of claim 11, wherein the nucleic acid comprises a DNA encoding RRSP and further comprises a promoter, wherein the promoter is a cell-specific promoter.

15. The method of claim 14, wherein the cell-specific promoter is a cancer-specific promoter.

16. The method of claim 10, wherein the cell is selected from a lung cancer cell, a colon cancer cell, a pancreatic cancer cell, a skin cancer cell, and a breast cancer cell.

17. A method of treating a cancer in a subject in need thereof, the method comprising administering to the subject a therapeutically effective amount of the system of claim 1.

18. The method of claim 17, wherein the cancer is selected from lung cancer, colon cancer, pancreatic cancer, skin cancer, and breast cancer.

19. The method of claim 17, wherein the nucleic acid comprises a DNA encoding RRSP and further comprises a promoter, wherein the promoter is a cell-specific promoter.

20. The method of claim 17, wherein the cancer is characterized by an increased rate of RAS mutation or increased signal flow through RAS.

21. The method of claim 17, wherein the pharmaceutical composition is administered intratumorally.

22. The method of claim 17, wherein the pharmaceutical composition is administered systemically.


SEQUENCE LISTING (13 sequences)

SEQ ID NO: 1 - RNA, 720 nt, synthetic construct
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - RNA, 1563 nt, synthetic construct
  source 1..1563: mol_type = other RNA
atgtacccatacgatgttccagattacgctggtgataaaaccaaggtcgtggtcgatttagcgcaaatctttacggtgcaagagctgaaagaaagagcaaaagtttttgctaaaccgattggcgcatcctaccaaggtattctcgatcaactcgaccttgtgcatcaggctaaaggccgcgatcaaatcgcagcgagctttgagcttaataagaagattaatgactacatcgctgaacatccaacttcggggcgtaatcaagcgctaacgcagttgaaagagcaggtcaccagtgcgttgtttatcggtaagatgcaagttgcccaagcgggtattgatgcaatcgcacaaacaagaccggagcttgccgctcgtatctttatggtcgcgattgaagaagccaacggtaaacacgtaggtttgacggacatgatggttcgttgggccaatgaagacccatacttggcaccgaagcatggttacaaaggcgaaacgccaagtgaccttggttttgatgcgaagtaccacgtagatctaggtgagcattacgctgatttcaaacagtggttagaaacgtcccagtcgaacgggttgttgagtaaagcgacgttggatgaatccactaaaacggttcatcttggctatagctatcaagaacttcaggatttgacgggtgctgaatcggtgcaaatggcgttctacttcctgaaagaagcggcgaagaaagcggatccgatttctggtgattcagctgaaatgatactgctgaagaaatttgcagatcaaagctacttatctcaacttgattccgaccgaatggatcaaattgaaggtatctaccgcagtagccatgagacggatattgacgcttgggatcgtcgttactctggtacaggctatgatgagctgacgaataagcttgctagtgcaacgggcgttgacgagcagcttgcggttcttctggatgatcgtaaaggcctcttgattggtgaagtgcatggcagcgacgtcaacggcctacgctttgttaatgaacagatggatgcactgaaaaaacagggagtcacagtcattggccttgagcatttacgctcagaccttgcgcaaccgctgattgatcgctacctagctacgggtgtgatgtcgagtgaactaagcgcaatgctgaaaacaaagcatctcgatgtcactctttttgaaaacgcacgtgctaacggtatgcgcatcgtcgcgctggatgcaaacagctctgcgcgtccaaatgttcagggaacagaacatggtctgatgtaccgtgctggtgctgcgaacaacattgcggtggaagtattacaaaatctgcctgatggcgaaaagttcgttgctatctacggtaaagcgcatttgcagtctcacaaagggattgaagggttcgttcctggtatcacgcaccgtctcgatcttcctgcgcttaaagtcagtgactcgaaccagttcacagttgaacaagacgatgtaagtctacgtgttgtctacgatgatgttgctaacaaaccgaagatcacgttcaagggcagtttgtag

SEQ ID NO: 3 - RNA, 1563 nt, synthetic construct
  source 1..1563: mol_type = other RNA
atgtacccatacgttccagattacgctatgggtgataaaaccaaggtcgtggtcgatttagcgcaaatctttacggtgcaagagctgaaagaaagagcaaaagtttttgctaaaccgattggcgcatcctaccaaggtattctcgatcaactcgaccttgtgcatcaggctaaaggccgcgatcaaatcgcagcgagctttgagcttaataagaagattaatgactacatcgctgaacatccaacttcggggcgtaatcaagcgctaacgcagttgaaagagcaggtcaccagtgcgttgtttatcggtaagatgcaagttgcccaagcgggtattgatgcaatcgcacaaacaagaccggagcttgccgctcgtatctttatggtcgcgattgaagaagccaacggtaaacacgtaggtttgacggacatgatggttcgttgggccaatgaagacccatacttggcaccgaagcatggttacaaaggcgaaacgccaagtgaccttggttttgatgcgaagtaccacgtagatctaggtgagcattacgctgatttcaaacagtggttagaaacgtcccagtcgaacgggttgttgagtaaagcgacgttggatgaatccactaaaacggttcatcttggctatagctatcaagaacttcaggatttgacgggtgctgaatcggtgcaaatggcgttctacttcctgaaagaagcggcgaagaaagcggatccgatttctggtgattcagctgaaatgatactgctgaagaaatttgcagatcaaagctacttatctcaacttgattccgaccgaatggatcaaattgaaggtatctaccgcagtagccatgagacggatattgacgcttgggatcgtcgttactctggtacaggctatgatgagctgacgaataagcttgctagtgcaacgggcgttgacgagcagcttgcggttcttctggatgatcgtaaaggcctcttgattggtgaagtgcatggcagcgacgtcaacggcctacgctttgttaatgaacagatggatgcactgaaaaaacagggagtcacagtcattggccttgagcatttacgctcagaccttgcgcaaccgctgattgatcgctacctagctacgggtgtgatgtcgagtgaactaagcgcaatgctgaaaacaaagcatctcgatgtcactctttttgaaaacgcacgtgctaacggtatgcgcatcgtcgcgctggatgcaaacagctctgcgcgtccaaatgttcagggaacagaacatggtctgatgtaccgtgctggtgctgcgaacaacattgcggtggaagtattacaaaatctgcctgatggcgaaaagttcgttgctatctacggtaaagcgcatttgcagtctcacaaagggattgaagggttcgttcctggtatcacggcgcgtctcgatcttcctgcgcttaaagtcagtgactcgaaccagttcacagttgaacaagacgatgtaagtctacgtgttgtctacgatgatgttgctaacaaaccgaagatcacgttcaagggcagtttgtag

SEQ ID NO: 4 - RNA, 1536 nt, synthetic construct
  source 1..1536: mol_type = other RNA
atgggtgataaaaccaaggtcgtggtcgatttagcgcaaatctttacggtgcaagagctgaaagaaagagcaaaagtttttgctaaaccgattggcgcatcctaccaaggtattctcgatcaactcgaccttgtgcatcaggctaaaggccgcgatcaaatcgcagcgagctttgagcttaataagaagattaatgactacatcgctgaacatccaacttcggggcgtaatcaagcgctaacgcagttgaaagagcaggtcaccagtgcgttgtttatcggtaagatgcaagttgcccaagcgggtattgatgcaatcgcacaaacaagaccggagcttgccgctcgtatctttatggtcgcgattgaagaagccaacggtaaacacgtaggtttgacggacatgatggttcgttgggccaatgaagacccatacttggcaccgaagcatggttacaaaggcgaaacgccaagtgaccttggttttgatgcgaagtaccacgtagatctaggtgagcattacgctgatttcaaacagtggttagaaacgtcccagtcgaacgggttgttgagtaaagcgacgttggatgaatccactaaaacggttcatcttggctatagctatcaagaacttcaggatttgacgggtgctgaatcggtgcaaatggcgttctacttcctgaaagaagcggcgaagaaagcggatccgatttctggtgattcagctgaaatgatactgctgaagaaatttgcagatcaaagctacttatctcaacttgattccgaccgaatggatcaaattgaaggtatctaccgcagtagccatgagacggatattgacgcttgggatcgtcgttactctggtacaggctatgatgagctgacgaataagcttgctagtgcaacgggcgttgacgagcagcttgcggttcttctggatgatcgtaaaggcctcttgattggtgaagtgcatggcagcgacgtcaacggcctacgctttgttaatgaacagatggatgcactgaaaaaacagggagtcacagtcattggccttgagcatttacgctcagaccttgcgcaaccgctgattgatcgctacctagctacgggtgtgatgtcgagtgaactaagcgcaatgctgaaaacaaagcatctcgatgtcactctttttgaaaacgcacgtgctaacggtatgcgcatcgtcgcgctggatgcaaacagctctgcgcgtccaaatgttcagggaacagaacatggtctgatgtaccgtgctggtgctgcgaacaacattgcggtggaagtattacaaaatctgcctgatggcgaaaagttcgttgctatctacggtaaagcgcatttgcagtctcacaaagggattgaagggttcgttcctggtatcacgcaccgtctcgatcttcctgcgcttaaagtcagtgactcgaaccagttcacagttgaacaagacgatgtaagtctacgtgttgtctacgatgatgttgctaacaaaccgaagatcacgttcaagggcagtttgtag

SEQ ID NO: 5 - RNA, 720 nt, synthetic construct
  source 1..720: mol_type = other RNA
atggtgagcaagggcgaggaggacaacatggccatcatcaaggagttcatgcggttcaaggtgcacatggagggcagcgtgaacggccacgagttcgagatcgagggcgagggcgagggccggccctacgagggcacccagaccgccaagctgaaggtgaccaagggcggccccctgcccttcgcctgggacatcctgagcccccagttcatgtacggcagcaaggcctacgtgaagcaccccgccgacatccccgactacctgaagctgagcttccccgagggcttcaagtgggagcgggtgatgaacttcgaggacggcggcgtggtgaccgtgacccaggacagcagcctgcaggacggcgagttcatctacaaggtgaagctgcggggcaccaacttccccagcgacggccccgtgatgcagaagaagaccatgggctgggaggccagcagcgagcggatgtaccccgaggacggcgccctgaagggcgagatcaagcagcggctgaagctgaaggacggcggccactacgacgccgaggtgaagaccacctacaaggccaagaagcccgtgcagctgcccggcgcctacaacgtgaacatcaagctggacatcaccagccacaacgaggactacaccatcgtggagcagtacgagcgggccgagggccggcacagcaccggcggcatggacgagctgtacaagagcggcaactga

SEQ ID NO: 6 - DNA, 5660 bp, synthetic construct
  source 1..5660: mol_type = other DNA
gtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagccgccaccatgtttatcggtaagatgcaagttgcccaagcgggtattgatgcaatcgcacaaacaagaccggagcttgccgctcgtatctttatggtcgcgattgaagaagccaacggtaaacacgtaggtttgacggacatgatggttcgttgggccaatgaagacccatacttggcaccgaagcatggttacaaaggcgaaacgccaagtgaccttggttttgatgcgaagtaccacgtagatctaggtgagcattacgctgatttcaaacagtggttagaaacgtcccagtcgaacgggttgttgagtaaagcgacgttggatgaatccactaaaacggttcatcttggctatagctatcaagaacttcaggatttgacgggtgctgaatcggtgcaaatggcgttctacttcctgaaagaagcggcgaagaaagcggatccgatttctggtgattcagctgaaatgatactgctgaagaaatttgcagatcaaagctacttatctcaacttgattccgaccgaatggatcaaattgaaggtatctaccgcagtagccatgagacggatattgacgcttgggatcgtcgttactctggtacaggctatgatgagctgacgaataagcttgctagtgcaacgggcgttgacgagcagcttgcggttcttctggatgatcgtaaaggcctcttgattggtgaagtgcatggcagcgacgtcaacggcctacgctttgttaatgaacagatggatgcactgaaaaaacagggagtcacagtcattggccttgagcatttacgctcagaccttgcgcaaccgctgattgatcgctacctagctacgggtgtgatgtcgagtgaactaagcgcaatgctgaaaacaaagcatctcgatgtcactctttttgaaaacgcacgtgctaacggtatgcgcatcgtcgcgctggatgcaaacagctctgcgcgtccaaatgttcagggaacagaacatggtctgatgtaccgtgctggtgctgcgaacaacattgcggtggaagtattacaaaatctgcctgatggcgaaaagttcgttgctatctacggtaaagcgcatttgcagtctcacaaagggattgaagggttcgttcctggtatcacgcaccgtctcgatcttcctgcgcttaaagtcagtgactcgaaccagttcacagttgaacaagacgatgtaagtctacgtgttgtctacgatgatgttgctaacaaaccgaagatcacgttcaagggcagtttggtcgacggtaccgcgggcccgggatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 7 - DNA, 1263 bp, synthetic construct
  source 1..1263: mol_type = other DNA
atgtttatcggtaagatgcaagttgcccaagcgggtattgatgcaatcgcacaaacaagaccggagcttgccgctcgtatctttatggtcgcgattgaagaagccaacggtaaacacgtaggtttgacggacatgatggttcgttgggccaatgaagacccatacttggcaccgaagcatggttacaaaggcgaaacgccaagtgaccttggttttgatgcgaagtaccacgtagatctaggtgagcattacgctgatttcaaacagtggttagaaacgtcccagtcgaacgggttgttgagtaaagcgacgttggatgaatccactaaaacggttcatcttggctatagctatcaagaacttcaggatttgacgggtgctgaatcggtgcaaatggcgttctacttcctgaaagaagcggcgaagaaagcggatccgatttctggtgattcagctgaaatgatactgctgaagaaatttgcagatcaaagctacttatctcaacttgattccgaccgaatggatcaaattgaaggtatctaccgcagtagccatgagacggatattgacgcttgggatcgtcgttactctggtacaggctatgatgagctgacgaataagcttgctagtgcaacgggcgttgacgagcagcttgcggttcttctggatgatcgtaaaggcctcttgattggtgaagtgcatggcagcgacgtcaacggcctacgctttgttaatgaacagatggatgcactgaaaaaacagggagtcacagtcattggccttgagcatttacgctcagaccttgcgcaaccgctgattgatcgctacctagctacgggtgtgatgtcgagtgaactaagcgcaatgctgaaaacaaagcatctcgatgtcactctttttgaaaacgcacgtgctaacggtatgcgcatcgtcgcgctggatgcaaacagctctgcgcgtccaaatgttcagggaacagaacatggtctgatgtaccgtgctggtgctgcgaacaacattgcggtggaagtattacaaaatctgcctgatggcgaaaagttcgttgctatctacggtaaagcgcatttgcagtctcacaaagggattgaagggttcgttcctggtatcacgcaccgtctcgatcttcctgcgcttaaagtcagtgactcgaaccagttcacagttgaacaagacgatgtaagtctacgtgttgtctacgatgatgttgctaacaaaccgaagatcacgttcaagggcagtttg

SEQ ID NO: 8 - protein, 494 aa, synthetic construct
QELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSL

SEQ ID NO: 9 - protein, 83 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFA

SEQ ID NO: 10 - protein, 83 aa, synthetic construct; identical to SEQ ID NO: 9

SEQ ID NO: 11 - protein, 83 aa, synthetic construct; identical to SEQ ID NO: 9

SEQ ID NO: 12 - protein, 83 aa, synthetic construct
LALHKVIMVGSGGVGKSALTLQFMYDEFVDEYDPTIEDSYRKKVVLDGEEVQIDILDTAGQEDYAAIRDNYFRSGEGFLCVFS

SEQ ID NO: 13 - protein, 83 aa, synthetic construct
MREYKLVVLGSGGVGKSALTVQFVQGIFVEKYDPTIEDSYRKQVEVDCQQCMLEILDTAGTEQFTAMRDLYMKNGQGFALVYS